SEQUENCE LISTING (19 sequences)

SEQ ID NO: 1 - DNA, 6930 bp, Homo sapiens
ggaaggagcaggaggcagcccaggcggagcgggaggagctggcagcggggcgcatgccaggcgggggtcctcagggcgccccagccgccgccggcggcggcggcgtgagccaccgcgcaggcagccgggattgcttaccacctgcagcgtgctttcggaggcggcggctggcacggaggccgggctacatgagaagctcgacagggcctgggatcgggttcctttccccagcagtgggcacactgttccggttcccaggaggggtgtctggcgaggagtcccaccactcggagtccagggccagacagtgtggccttgactcgagaggcctcttggtccggagccctgtttccaagagtgcagcagcccctactgtgacctctgtgagaggaacctcggcgcactttgggattcagctcagaggtggcaccagattgcctgacaggcttagctggccgtgtggccctgggagtgctgggtggcagcaagagtttgcagccatggatagttctgagaccctggacgccagctgggaggcagcctgcagcgatggagcaaggcgtgtccgggcagcaggctctctgccatcagcagagttgagtagcaacagctgcagccctggctgtggccctgaggtccccccaacccctcctggctctcacagtgcctttacctcaagctttagctttattcggctctcgcttggctctgccggggaacgtggagaagcagaaggctgcccaccatccagagaggctgagtcccattgccagagcccccaggagatgggagccaaagctgccagcttggacgggcctcacgaggacccgcgatgtctctctcagcccttcagtctcttggctacacgggtctctgcagacttggcccaggccgcaaggaacagctccaggccagagcgtgacatgcattctttaccagacatggaccctggctcctccagttctctggatccctcactggctggctgtggtggtgatgggagcagcggctcaggggatgcccactcttgggacaccctgctcaggaaatgggagccagtgctgcgggactgcctgctgagaaaccggaggcagatggaggtaatatccttaagattaaaacttcagaaacttcaggaagatgcagttgagaatgatgattatgataaagctgagacgttacaacaaagattagaagacctggaacaagagaaaatcagcctgcactttcaacttccttcaaggcagccagctcttagcagtttcctgggtcacctggcagcacaagtccaggctgccttgcgccgtggggccactcagcaggccagcggagatgacacccacaccccactgagaatggagccgaggctgttggaacccactgctcaggacagcttgcacgtgtccatcacgagacgagactggcttcttcaggaaaagcagcagctacagaaagaaatcgaagctctccaagcaaggatgtttgtgctggaagccaaagatcaacagctgagaagggaaatagaggagcaagagcagcaactccagtggcagggctgcgacctgaccccactggtgggccagctgtccctgggtcagctgcaggaggtcagcaaggccttgcaggacaccctggcctcagccggtcagattcccttccatgcagagccaccggaaaccataaggagcctccaggaaagaataaaatccctcaacttgtcacttaaagaaatcactactaaggtgtgtatgagtgagaaattctgcagcaccctgaggaagaaagttaacgatattgaaacccaactaccagccttgcttgaagccaaaatgcatgccatatcaggaaaccatttctggacggctaaagacctcaccgaggagattagatcattaacatcagagagagaagggctggagggactcctcagcaagctgttggtgttgagttccaggaatgtcaaaaagctgggaagtgttaaagaagattacaacagactgagaagagaagtggagcaccaggagactgcctatgaaacaagtgtgaaggaaaatactatgaagtacatggaaacacttaagaataaactgtgcagctgcaagtgtccactgcttgggaaagtgtgggaagctgacttggaagcttgtcgattgcttatccagagcctacagctccaggaagccaggggaagcctgtctgtagaagatgagaggcagatggatgacttagagggagctgctcctcctattccccccaggctccactccgaggataaaaggaagacccctttgaaggtattggaagaatggaagactcacctcatcccctctctgcactgtgctggaggtgaacagaaagaggaatcttacatcctttctgcagaacttggagaaaagtgtgaagacataggcaagaagctattgtacttggaagatcaacttcacacagcaatccacagtcatgatgaagatctcattcagtctctcaggagggagctccagatggtgaaggaaactctgcaggccatgatcctgcagctccagccagcaaaggaggcgggagaaagagaagctgcagcttcctgcatgacagctggtgtccacgaagcacaagcctgaggagtgacgggatgggggagggaggtgggccaccatgtttggacccggggggctgctcttccctcccccgccatagctaagatgcctgaatcaattacggagatacagagccttgaggtctttcagtggaaaggtggttcatgttcattctcatcagtgtgaaactgaggagtctgcaatttggaatatggagagagagactgatttgctgaatttccttctaaatgtcactcaaaaatttcttttccatgtcattcttgggaatgtcttccacaggatttgagaatagtttcatctcagcccccattagagagaagttggggtgaattctggaaaaatgtctctttttcctgtgccatttgccttctgctgcaacgaaaatatttcctgattcaagattctataaaaaggaaaccaagcataagactctgtcatcatacctgttacacgttcctacaggtgcacaatctaagagagctaattaacctcagagtctggagttaacagcttttcaccttacttctcctgtgatctaatattatcttagaaaaattaatatgcaatttccaaaagatattttggtaagacaacaacctcccagtgatatgccacctttcaattttccttttgtggcaatgattgcatctgaagaaaggatccctgagagtctctgtttcatcaggacattctgaaatttacccacagtgaggctgtggatggatcaggggacctgtataaaatgtttgagcctgttccattttcccgtggaacctgtttcactcaatgccaggcagtgcagcatttaggaaagcagtgcagtactcagtaaggcagtgcagtactcagtaacacaatacagtactcaggcagtgcagtactcagtaagacagtgcagtgctcagtaaggcagtgcaatactcagtaacagtgtagtactcagtaacagtgaagtactcagtaatacagtacagtattcagtaaggcagtgaagtactcagtaatacaatacagtactcagttaggcagtgcagtactcaggaatgcagcacagtactcaggcagtgcaatactcagtgcggtactcagtaacacagtgcagtactcagtaacagtgcagtactcagtaacagtgcagtactcagtaaggcagtgcagtactcagtaacacagtgcagtactcagtaatacagtacagtactcagtaaggcagtatggtactcagtaaagcaatgcaatgctcagtaacacagtgcagtgctcaataaggcagtgcagtgctcagtaaggcagtgaattgcttagtaacacagtgtagtgctcagtaggacagcatagtactcagtaacacagggcagctagtactcagtactataagtactgagtacttatataggcaatgtagtactcagtaaatcagtgcagtactcagtaatgcaagggcatttcaggctcctgctgggctgcttctttggcccagctgggactcctattgagacagctgcaaaacaggctgatttcaattaggcagcacttcccaaagtgcactgaggaaggtggccccaagagaagctctctaaacaaaggagtaccctctctggtcaagtacctttggtaaatacaccataccataatatctgcttggagaaccacaatgcacattagcatattagtctgagagagaacttatagtaaggaaactcacttgattttatctaacctcaaactttccaagtttaatggatcgtgaatttttttcatgtaactcctattcatatcccatagatctagtattgtacagcactgcattctctgaggaagtcccagtccaaactctgatttacatcactttagaaaccacactcacacttttgcagagtgttgagcttaataactacctgccacagattggtaaatttaatccagtggttgttctgtttgtgcttctgttctcatttatgtgtttagggatagtgaggttcctgccttcactaggatccacggatatgagaccatttttgtcatttcctgaagtcacactggcgtttccagaaggcatctggtgctttgctcagccttccatgctgtgcagcacttctgtcctcagtcaaggagatggccatgcttaagccagcaattggctggggtccaggaaacaaagcaaaagcacaatatgtgaatgtgctgattgtgttccctatggctttatctcgagcaaaatacactctacatattttaataataagtataattagcttgttcctggacttcattttcaatgatgaaccaaattcctgaattatttataattgtgtctaaagaaaattatgaactggtcacatggcacttggaatccttgagttaattccagtgaagcaaaacttgggaagagtcaggattggccacattgccaataacaaattcctacttcgacatatgtcttttcaaaaagcctcccagacacaagacatcttaaccgtcactagcccaagtgttttgtattactcagacaccatcatgaaataattctgtgaggtcatgatgtatttgaaaattctgcaagttaataactgccttgaattgtttgaacccgaaataagggttctttggtacctctagtagatagtgtgttcatttccctgctgcaaattttgaagtatttgggcaggtgagtcatgttttaaccacaagccataactcatctgttgtctttgcttggtcttagagtatcattcagaaagtccgctaagggccagcgtgcttcttctggctacacaaccttctcaggacaagcccactgtcttaagccactttgaccctgggagacacaggactgtgtatcctcaatcatactatacagcagtttttgtcaggggaacataaaaatatccaagagaggttagggcttagatttaaaagcatcaaaacaacaacaatggaaatttatgttggcgatagccaagaccacaagcaaaagcacatactggaaatgatgagttagaatctgatttgactgggatgttttatgagaatgtaagtgtgatattatactgtctgccttgctggaatgctggctttcaaatggtcacccatttttctttcactggcctgagttaggacatgctatcagtaatagtcccagttccatccaactttctgaaatttcatttttttttttgagatggagtctctctctgtcacccaagttggagtgcagtggccccgcaatctcggctcactgcaacctctacctcccaggttcaagctattctactgcctcagcctcccaagtagctggggttacaggcatttgccaccgggccctgatgatttttgtatttttagtagagacagggcttcaccatgttggctaggagggtctcaaactcctgacctcaggcgatccacccccacctcggcttcccaaagtgctgagattacaggtgtgagccaccgcacccggccaactttctgaaatttcaaaactgaattgatccttctccaaattagtatatactattggaaacttgtctttccctgcagtaaggctggtttccccaccccagaaacatgtaacggttggtaccatgctaagcccttgccatgctaagccctttacagtcatatcctataatccccatatcaaccttataaggaaggtgtttgtagatgatgcaactgagccttaagaggactaattccctttttctaaggcacagagctggtaaaatgtgaagtaatagtgaacctaacagtcagagacaggcagcatgctcttaactagtgctcttcctaaagttcctttaatgtccttttgagattttgagccatggaacttacttgttcacctggctaagaactcatggccactgtggaaatcttggttagggagtcaaagaaactgagcctggggcaaacgaggcttcccacactgccaggggagcctcactgtgaagtctaggctcagacaggcatcaacaaacctattcaccccaccatcatcctgatctaaccattccccagtcatcccaggaaaaccactcacagcctgacactgggctgactttcttgaagatcctcatccaattggtgtttttcagaagtgttccaatattatgaattctgtgttgtggagaaaagcaaccatgcatttactggtcaatgccttcttgtatatgtaattcaatacttttacttttaatatcctcaccttatctaatctttgaattttgtcatgtaatttattgcttcattaaggttactttttgttatacaaaataaaagctgatatccaaggcaaaaaaaaaaaaaaaaa

SEQ ID NO: 2 - protein, 854 aa, Homo sapiens
MPGGGPQGAPAAAGGGGVSHRAGSRDCLPPAACFRRRRLARRPGYMRSSTGPGIGFLSPAVGTLFRFPGGVSGEESHHSESRARQCGLDSRGLLVRSPVSKSAAAPTVTSVRGTSAHFGIQLRGGTRLPDRLSWPCGPGSAGWQQEFAAMDSSETLDASWEAACSDGARRVRAAGSLPSAELSSNSCSPGCGPEVPPTPPGSHSAFTSSFSFIRLSLGSAGERGEAEGCPPSREAESHCQSPQEMGAKAASLDGPHEDPRCLSQPFSLLATRVSADLAQAARNSSRPERDMHSLPDMDPGSSSSLDPSLAGCGGDGSSGSGDAHSWDTLLRKWEPVLRDCLLRNRRQMEVISLRLKLQKLQEDAVENDDYDKAETLQQRLEDLEQEKISLHFQLPSRQPALSSFLGHLAAQVQAALRRGATQQASGDDTHTPLRMEPRLLEPTAQDSLHVSITRRDWLLQEKQQLQKEIEALQARMFVLEAKDQQLRREIEEQEQQLQWQGCDLTPLVGQLSLGQLQEVSKALQDTLASAGQIPFHAEPPETIRSLQERIKSLNLSLKEITTKVCMSEKFCSTLRKKVNDIETQLPALLEAKMHAISGNHFWTAKDLTEEIRSLTSEREGLEGLLSKLLVLSSRNVKKLGSVKEDYNRLRREVEHQETAYETSVKENTMKYMETLKNKLCSCKCPLLGKVWEADLEACRLLIQSLQLQEARGSLSVEDERQMDDLEGAAPPIPPRLHSEDKRKTPLKVLEEWKTHLIPSLHCAGGEQKEESYILSAELGEKCEDIGKKLLYLEDQLHTAIHSHDEDLIQSLRRELQMVKETLQAMILQLQPAKEAGEREAAASCMTAGVHEAQA

SEQ ID NO: 3 - DNA, 1613 bp, Homo sapiens
gtccggctgagccccgggatccgcctccctccgccaggacccgcacagataaactcatcctgaaagtcgctgttgttctcctgctgagcaagaatggaggccccactggtgagtctggatgaagagtttgaggaccttcgaccctcctgctcggaggacccggaggagaagccccagtgtttctatggttcatctccccaccatctcgaggacccctccctctccgagcttgagaatttttcttccgaaataatcagcttcaagtccatggaggacctcgtaaatgaatttgatgagaagctcaatgtctgctttcggaactacaacgccaagaccgagaacctagctcccgtgaagaaccagttacagatccaagaggaggaggagacccttcaggacgaggaggtttgggatgctctgacagacaattacatcccttcactctcagaagactggagggatccaaacatcgaggctctgaatggcaactgctctgacactgagatccatgagaaagaagaggaagagttcaatgagaagagtgaaaatgattccggtatcaacgaggagcctctgctcacagcagatcaggtaattgaggagattgaggaaatgatgcagaactccccagaccctgaggaagaagaggaggttctggaagaagaggatggaggagaaacttcctcccaggcagactcggtcctcctgcaggagatgcaggcattgacacagaccttcaacaacaactggtcctatgaagggctgaggcacatgtctgggtctgagctgaccgagctgctggaccaggtggagggtgccatccgtgacttctcggaggagctggtgcagcagctggcccgccgggacgagctggagtttgagaaggaagtgaagaactcctttatcacggtgcttattgaggttcagaacaagcagaaggagcagcgagaactgatgaaaaagaggcggaaagagaaagggctgagcctgcagagcagccggatagagaagggaaaccagatgcctctcaagcgcttcagcatggaaggcatctccaacattctgcagagtggcatccgccagacctttggctcctcaggaactgacaaacagtatctgaacacagtcattccttacgagaagaaagcctctcctccctcagtggaagacctgcagatgctgacaaacattctctttgccatgaaggaggataatgagaaggtgcctactttgctaacggactacattttaaaagtgctctgccctacctaaccttgccctttggagcagcctcgctgcaggaggtcactgagcaagagtcattccatcacagggactgcatgagaccatgtaacctccgacatgtatttaaacgtgtatagcttaacctggattaaacacgagcaagcgcgcggggtcctttgccgttggcttctagtgctagtaatcattggatgcatgatggggcagggccggtgatggtgcctcccccttgctggtgtcaggagaggggaaggcagccgctttcaccgctcattatgtagtctggctacagccctcaaaaacagcttatactcttaagactaattttgaaataaaaccttcatttaatt

SEQ ID NO: 4 - protein, 392 aa, Homo sapiens
MEAPLVSLDEEFEDLRPSCSEDPEEKPQCFYGSSPHHLEDPSLSELENFSSEIISFKSMEDLVNEFDEKLNVCFRNYNAKTENLAPVKNQLQIQEEEETLQDEEVWDALTDNYIPSLSEDWRDPNIEALNGNCSDTEIHEKEEEEFNEKSENDSGINEEPLLTADQVIEEIEEMMQNSPDPEEEEEVLEEEDGGETSSQADSVLLQEMQALTQTFNNNWSYEGLRHMSGSELTELLDQVEGAIRDFSEELVQQLARRDELEFEKEVKNSFITVLIEVQNKQKEQRELMKKRRKEKGLSLQSSRIEKGNQMPLKRFSMEGISNILQSGIRQTFGSSGTDKQYLNTVIPYEKKASPPSVEDLQMLTNILFAMKEDNEKVPTLLTDYILKVLCPT

SEQ ID NO: 5 - DNA, 2478 bp, Rattus sp.
  misc_feature (557)..(557): n is a, c, g, or t
atgcagggcgggggtccccggggcgcttggatccacagtccgagccactgcccaggcaatgggcatggctcgccgcctgcagtagcccctcagaggcggcggctgacacggagaccaggctacatgagaagcacagccagtcctgggatcgggttcctgtctccagcagtgggcatgccacgtccgatctcagcggggctgacaggccaggagttctacccctcacagtccaaggctagacagtgcagccttgacctcagaagccactgccaagactcattggtgggcaacccttttctcaagggctcccttggccctgctgtgacctctgtgggccacctgcacccagcccagggctctatgagagaaaggacggtgcactctggggttcattccgggaatgacagaagacaatccgagaggctgactggggacagtggttgtcggcaagaatttttatcttcagacagctctaagtctctggcttctaaccttgatgtaacttggagcaagggatcgaggggcctgaagactgtgaggcctctggtatcaccggngtcnaatggccccgntgacatcccatcccttcctgggttccagacaccttttactttcaactttcaaanttcatccgactctccctncgaggctgctggagaacgcggagaagcagaaggttgcctgccatccagagaggccgaacccctgcatcagagcccccaagagatggcagctgaaggatccggctcagacaggccccacggggagcctcggcatctctggaccttcagtcttcacgctgctccgggcttggtggacttggctcaggggacgaggagcaacaggcaaccagaatgtggcatggtctcctcctccgatgctggcttctcttcccaggatgcatcccccgctggagggcggagcgaccaggacggaggctgggcggatgcccatggatggcacgctctgctcagggaatgggagcctatgctgcaggactacctactgagcaaccgcaggcagctggaggtcacttccttgattttaaagcttcagaaacttcaagaaaaagcggtcgaggatggcgattacgacatggcagagacattgagacagaggttggaagacctggaacaggagaaaggccgcctgccctgggctctgccctcacagcaaccagcccttcgcagcttcttggggtacctggcgacacagacacacgcggccttgcatggagccccccaaagggctggcagcgatgacccagaagccccacttgagggacagcggaggaccaccgcccaggatagcctgccgggactagccgtcaccaggcgggactggctgatgcgagagaaagagcaattgcagaaggaaatcgaagctctccgggcaagggtgtctgtgctggaggcgaaggaacaacggctgagccaagagttggaggatcaggagatgctgctccggtggcagggctgtgaccagatggcactggtggcacagctgtccccaggccagctgcaggaagtcagcaaggccttgggagagactctgacctctgccagatgggctcccttccgcgtggagccacctgagaccctcaggagcctccgggaaaggacaaagtcactgagcctggctgttagagaactcactgagcaggtatgctcaggtgagaagctatgcagctctctgaggaagagactcgctgaccttgacaccaggctgcctgccttgctggaagccaagatgctggccctatcaagaagctgcttctctacagccaaggagctcgcaaaggagatttgggccgtgtcatcggagcgggaagggctagagatgttcctgggcaggctgctggccctcagctcccggaacacgagaagactgggcagcgtcaaagaggactacctcaggtgcaggcaggacctggccctcccggaagccgcccacaaaacacgcgtgaaggcaaacactgtgaagtgcacggaagtgttggagggtcagctgagctgctgcaggtgcccgctgcttgagagagtgtggaaagcggacttggaggcgtgtcaattgctgatgcaaagcccagagatccaggaagcaagcagcagctcacatgtggaggatgagaagcaggtgcacagcacaggagaggcggctcagacagctgccctggctgtccctagaacaccccaccccgaagaggaaaagtcccccttgcaggaatctcatgtcgtttttgctgaagttggagacaaatgcgaagccataggcataaggctcctgcacctggaagaccagcttctcggcgccatgcacggtcacgatgaagctctctttcagtctctccagggggagctccagatggttaaggaaacactacagaccatgttcctgcagctccagccagcaaaggaggcaggaggagaggcctcagcttcctattcgacagctggtgctcaggaaaccgaggcctga

SEQ ID NO: 6 - protein, 825 aa, Rattus sp.
  misc_feature (186)..(186): Xaa can be any naturally occurring amino acid
MQGGGPRGAWIHSPSHCPGNGHGSPPAVAPQRRRLTRRPGYMRSTASPGIGFLSPAVGMPRPISAGLTGQEFYPSQSKARQCSLDLRSHCQDSLVGNPFLKGSLGPAVTSVGHLHPAQGSMRERTVHSGVHSGNDRRQSERLTGDSGCRQEFLSSDSSKSLASNLDVTWSKGSRGLKTVRPLVSPXSNGPXDIPSLPGFQTPFTFNFQXSSDSPXEAAGERGEAEGCLPSREAEPLHQSPQEMAAEGSGSDRPHGEPRHLWTFSLHAAPGLVDLAQGTRSNRQPECGMVSSSDAGFSSQDASPAGGRSDQDGGWADAHGWHALLREWEPMLQDYLLSNRRQLEVTSLILKLQKLQEKAVEDGDYDMAETLRQRLEDLEQEKGRLPWALPSQQPALRSFLGYLATQTHAALHGAPQRAGSDDPEAPLEGQRRTTAQDSLPGLAVTRRDWLMREKEQLQKEIEALRARVSVLEAKEQRLSQELEDQEMLLRWQGCDQMALVAQLSPGQLQEVSKALGETLTSARWAPFRVEPPETLRSLRERTKSLSLAVRELTEQVCSGEKLCSSLRKRLADLDTRLPALLEAKMLALSRSCFSTAKELAKEIWAVSSEREGLEMFLGRLLALSSRNTRRLGSVKEDYLRCRQDLALPEAAHKTRVKANTVKCTEVLEGQLSCCRCPLLERVWKADLEACQLLMQSPEIQEASSSSHVEDEKQVHSTGEAAQTAALAVPRTPHPEEEKSPLQESHVVFAEVGDKCEAIGIRLLHLEDQLLGAMHGHDEALFQSLQGELQMVKETLQTMFLQLQPAKEAGGEASASYSTAGAQETEA

SEQ ID NO: 7 - DNA, 1697 bp, Rattus sp.
tagctggcttgcccgctgccgttgcactgcaccgggccacctctagccggtggatggggatgtctggctgaaccccgggatccgattccctcctccaggacctgctcagggaaacggatcttgacagttgccatcattgtactacggtgccaggatggaggctccactggtgagtctggatgaagagtttgaagatatcaggccctgctgcactgaggacccagaggaaaagccacaaagtctctatggcacatctcctcaccacctggaggacccttccctctccgagcttgagaatttttcttccgaaataatcagcttcaagtccatggaggacctcgtgaatgaatttgatgagaagctcaatgtctgctttcggaactacaatgccaagaccgagaacctggctccagtgaagaaccagttgcagatccaggaggaggaggagactcttcgggatgaggaggtttgggatgctctgacagacaattacatcccttcactctcagaagactggagggacccaaacattgaagctctgaatggcaacagctctgacactgagatccacgagaaggaagaagaagatgagttcattgagaagagtgaaaacgactctggtatcaacgaggagcctctcctcacagctgaccaggtgattgaggagatcgaagaaatgatgcagaactccccagatcctgaggaggaagtggaggtgctggaagaggaggatggaggagaaatctcgtctcaggcagattcagtcctgctacaagagatgcaggccctgacgcagaccttcaacaacaactggtcctatgaagggctgagacacatgtctgggtctgagctgacagagctgctggaccaggtggagggtgccatccgtgacttctcggaggagctggtccaccagctggcccgcagagatgagctggagtttgagaaggaagtgaagaactctttcatcacggtgctcattgaggttcagaataagcagaaggagcagcgggaactgatgaagaagaggcggaaagagaaggggttaagcctgcagagcagtcggatagagaaggggaaccagatgcctctcaagcgtttcagcatggagggcatctccaacatcctgcagagtggcatccgccagacctttggctcctcaggagctgacagacagtacctgaacacagtcatcccttatgagaagaaatcttctcctccctctgtggaagacctgcagatgctgacaaacattctcttcgccatgaaggaggataacgagaaggtgcctactttgctaacggactacattttaaaagtgctctgtccaacctaacctcacctttggagcagcctcactgcaggaggtcactgagcaagaaagtcattccatcactgggattgcatgacaccacgtaacctccggcgtgtatttaaacgtgtatagcttaacctggattaaacatgagcaaccgcgcacgcgcggggtcctttgctgttggcttctagtgctagtaatcattggatgcatgatcgggcgtagggccagtgactctgctccctctaacctgtgttggggaagacaggtgccctcaccactcattatgtagtctggctccagccctcaacccagcttatactcttcagactaattttgaaataaaccttcatttaattaatatcaaaaaaaaaaaaaa

SEQ ID NO: 8 - protein, 393 aa, Rattus sp
MEAPLVSLDEEFEDIRPCCTEDPEEKPQSLYGTSPHHLEDPSLSELENFSSEIISFKSMEDLVNEFDEKLNVCFRNYNAKTENLAPVKNQLQIQEEEETLRDEEVWDALTDNYIPSLSEDWRDPNIEALNGNSSDTEIHEKEEEDEFIEKSENDSGINEEPLLTADQVIEEIEEMMQNSPDPEEEVEVLEEEDGGEISSQADSVLLQEMQALTQTFNNNWSYEGLRHMSGSELTELLDQVEGAIRDFSEELVHQLARRDELEFEKEVKNSFITVLIEVQNKQKEQRELMKKRRKEKGLSLQSSRIEKGNQMPLKRFSMEGISNILQSGIRQTFGSSGADRQYLNTVIPYEKKSSPPSVEDLQMLTNILFAMKEDNEKVPTLLTDYILKVLCPT

SEQ ID NO: 9 - DNA, 3163 bp, Mus sp.
cagtcaccgccgtccggggcgcagccggccgcacgatgcagggcgggggtccccgggacgctccgatccacagtccgagccacggcgcagacagtgggcatggcttaccgcctgcagtagcccctcagaggcggcggctgacacggagaccaggctacatgagaagcacagcgggttctgggatcgggttcctctctccagcagtgggcatgccacacccgagctcagcagggctgacaggccagcagtcccaacactcacagtccaaggctgggcagtgcggacttgaccctgggagccactgccaagcctcactggtgggcaagccttttctcaagagctcccttgtccctgctgtggcctctgagggccacctgcacccagcccagcgctctatgagaaaaagaccagtgcactttggggttcattccaagaatgacagtagacaatctgagaagctgactgggtcatttaagcctggggacagtgggtgttggcaagaattattatcttcagacagctttaagtctctggctcctagccttgatgcaccctggaacacgggatcaaggggcctgaagactgtgaaacctctggcatcatcggcgttgaatggccccgctgatatcccatcccttcccggcttccaagacacctttacttccagcttcagcttcatccaactctcccttggtgctgctggagaacgcggagaagcagaaggttgcctgccatccagagaggccgaacctctgcatcagaggccccaagagatggcagctgaagcatctagctcagacaggccccatggggaccctcggcatctctggaccttcagtcttcacgctgctccaggcttggcagacttggctcaggtgacgaggagcagcagcaggcaaccagaatgtggcacggtctcctcctcctcggatactgtcttctcttcccaggatgcatcctccgctggtgggcggggcgaccagggcggcggctgggccgatgcccatggatggcatacattgctcagggaatgggagcccatgctgcaggactacctactgagcaaccgcaggcagctggaggtcacttccttaattttaaagcttcagaaatgtcaagaaaaagcggtcgaggatggcgattacgatactgcagagacattgagacagaggttggaagaactggaacaggagaaaggccacctgtcctgggctctgccttcacagcaacctgctcttcgcagcttcttgggttacctggcagcacagatacaggtggccttgcatggagccacccaaagggccggcagcgatgatccagaagccccacttgaaggacagctgaggactaccgcccaggatagcctgcctgcatccatcaccaggagggactggcttattcgagagaaacagcaattgcagaaggaaatcgaagctctccaagcacggatgtctgcgctggaggcaaaggaaaaacggctgagccaagagttggaggagcaggaggtgctgctccggtggccaggctgtgacctgatggcactggtggcccagatgtccccaggccagctgcaggaggtcagcaaggccttgggagagaccctgacctctgccaaccaggctcccttccacgtggagccacctgagaccctcaggagcctccgggaaaggacaaaatcattgaacctggctgtcagagaactcactgctcaggtgtgctcaggtgagaagctgtgcagctctctgaggaggagactcagtgacctcgacaccaggctgcctgccttgctggaagccaagatgctggccctatcaggaagctgcttctccacagccaaggagctcacggaggagatttgggccttgtcgtcagagcgggaagggctagagatgttcctgggcaggctgttggcactcagctccaggaacagcagaaggctaggcatcctcaaagaggattacctcaggtgcaggcaggacctggcactccaggacgccgcccacaaaacacgcatgaaggcaaacactgtgaagtgcatggaagtgttggaaggtcagctgagcagctgcaggtgcccgctgcttgggagagtgtggaaagcagacttggagacttgtcagttgctaatgcagagcctgcagcttcaggaagcaggcagcagcccacacgcagaggacgaggagcaggtgcatagcacaggagaggccgcccagacagctgctctggctgtccctcgaacaccccaccctgaagaagaaaagtcccccttgcaggtgctccaggagtgggacacccactcagctctttcaccacactgtgctgcaggcccatggaaagaggattctcacatcgtttctgctgaagttggagaaaagtgcgaagccataggcgtgaggctcctacacctggaagaccagcttctcggggccatgtacagtcacgacgaagctctctttcagtctctccagggggagctccagacggtgaaggaaacactgcaggccatgatcctgcagctccagccaacaaaggaggcaggagaggcctcagcttcctatccgacagctggtgctcaggaaaccgaggcctgaggtcctgctctttctccattcgaaaccaaacatgaaatggccactatacctgccggggtctttctacagacactccaatgccaggagcccacaaacctcagagtctgtgcaggtccttagttccccaggccaattcctctgggggctctgctcagtctcaggagatgtctacaggatgcctcagcaaggtgcagcctttatgttccaaggcaatggctgcagctcacaaccactgctgccgctgccgctaccgctgccgctgctgccaccaccaccagcaccaccagaagtgcttgctgcggcatcaagtcattctgagattccagccaatgaggtgggaggcgagaatgtgaggagaggcatgggtgcaggaggagacaatggacacgttgagcctctccagggaattacgacgtcatggagcctgaagagcagtgtctattcactgaggcgatgtggactcagacaggcagctcactgctccataatggtgagccacaggttcctgtggattgctccctgggttcagcggaggctcctcctggggcagctgcaggcaggttgttcacctg

SEQ ID NO: 10 - protein, 851 aa, Mus sp.
MQGGGPRDAPIHSPSHGADSGHGLPPAVAPQRRRLTRRPGYMRSTAGSGIGFLSPAVGMPHPSSAGLTGQQSQHSQSKAGQCGLDPGSHCQASLVGKPFLKSSLVPAVASEGHLHPAQRSMRKRPVHFGVHSKNDSRQSEKLTGSFKPGDSGCWQELLSSDSFKSLAPSLDAPWNTGSRGLKTVKPLASSALNGPADIPSLPGFQDTFTSSFSFIQLSLGAAGERGEAEGCLPSREAEPLHQRPQEMAAEASSSDRPHGDPRHLWTFSLHAAPGLADLAQVTRSSSRQPECGTVSSSSDTVFSSQDASSAGGRGDQGGGWADAHGWHTLLREWEPMLQDYLLSNRRQLEVTSLILKLQKCQEKAVEDGDYDTAETLRQRLEELEQEKGHLSWALPSQQPALRSFLGYLAAQIQVALHGATQRAGSDDPEAPLEGQLRTTAQDSLPASITRRDWLIREKQQLQKEIEALQARMSALEAKEKRLSQELEEQEVLLRWPGCDLMALVAQMSPGQLQEVSKALGETLTSANQAPFHVEPPETLRSLRERTKSLNLAVRELTAQVCSGEKLCSSLRRRLSDLDTRLPALLEAKMLALSGSCFSTAKELTEEIWALSSEREGLEMFLGRLLALSSRNSRRLGILKEDYLRCRQDLALQDAAHKTRMKANTVKCMEVLEGQLSSCRCPLLGRVWKADLETCQLLMQSLQLQEAGSSPHAEDEEQVHSTGEAAQTAALAVPRTPHPEEEKSPLQVLQEWDTHSALSPHCAAGPWKEDSHIVSAEVGEKCEAIGVRLLHLEDQLLGAMYSHDEALFQSLQGELQTVKETLQAMILQLQPTKEAGEASASYPTAGAQETEA

SEQ ID NO: 11 - DNA, 28 bp, Artificial sequence
  primer
acactgagacagagattggaagacctgg

SEQ ID NO: 12 - DNA, 28 bp, Artificial sequence
  primer
tggcagaggtcagagtctctcccaaggc

SEQ ID NO: 13 - DNA, 1224 bp, Homo sapiens
  CDS (1)..(1224)
atgcaacagaaggcttttgaggaaagcagatatccctggcaggagtcctttgagaatgttgctgtgtgcctgccattacgctgcccgaggtgtggagaccataccagatttagaagcttgtcatccttgagggcccatctggagttcagtcacagctacgaagaaagaaccctcttgacaaaatgcagtctctttccatccctcaaagacacagacctagtcacttcctcagaactcctgaaaccgggaaaattgcagagcagtggcaacgtggtaaagcagaaaccgagctatgttaacttgtacagcatttcacatgaacattccaaggacaggaagccatttgaggtggtggcagagaggcctgtgtcctatgtgcagacctacactgccatggacctccatgcagactcgctggatgggacacggtcgggtcctggactgcccacctcagacaccaaagcttctttcgaggcacatgtcagagaaaaattcaatcgaatggttgaggctgtggataggaccattgagaagagaattgataaactcaccaaagagttggcccagaaaactgcggaactgttggaagttcgggcagcttttgtgcagctgactcagaaaaagcaggaagttcagagacgagagcgggccttaaacagacaggtggacgtggccgtggaaatgatagctgtactgaggcaacgcctgacggaatctgaggaggagcttcttaggaaagaagaagaagttgtcacattcaaccatttcctggaagcggcagctgagaaggaggttcaagggaaagcccggctccaggactttattgagaatctgttacagcgggtagaactggcggagaagcagcttgagtactatcagagccagcaggcctctggctttgtccgtgatctcagcgggcacgtgcttacagacatctcctcaaataggaagcccaaatgcctaagccgagggcacccgcattcggtatgtaaccaccctgatctcaagtcccatttccacccaaagggaaggaaccacctgaaaaaggccaaggatgacagagccagcatgcagcctgccaaggccattcacgaacaggctgagtcctcaagagacctctgcagacctccaaagaaaggggagctcctggggtttggccgcaaaggcaacatcaggcccaaaatggctaaaaaaaagccaacagccattgtgaacatcatctaa

SEQ ID NO: 14 - protein, 407 aa, Homo sapiens
MQQKAFEESRYPWQESFENVAVCLPLRCPRCGDHTRFRSLSSLRAHLEFSHSYEERTLLTKCSLFPSLKDTDLVTSSELLKPGKLQSSGNVVKQKPSYVNLYSISHEHSKDRKPFEVVAERPVSYVQTYTAMDLHADSLDGTRSGPGLPTSDTKASFEAHVREKFNRMVEAVDRTIEKRIDKLTKELAQKTAELLEVRAAFVQLTQKKQEVQRRERALNRQVDVAVEMIAVLRQRLTESEEELLRKEEEVVTFNHFLEAAAEKEVQGKARLQDFIENLLQRVELAEKQLEYYQSQQASGFVRDLSGHVLTDISSNRKPKCLSRGHPHSVCNHPDLKSHFHPKGRNHLKKAKDDRASMQPAKAIHEQAESSRDLCRPPKKGELLGFGRKGNIRPKMAKKKPTAIVNII

SEQ ID NO: 15 - DNA, 2589 bp, Artificial
  Synthetic DISC1-HA
atgccaggcgggggtcctcagggcgccccagccgccgccggcggcggcggcgtgagccaccgcgcaggcagccgggattgcttaccacctgcagcgtgctttcggaggcggcggctggcacggaggccgggctacatgagaagctcgacagggcctgggatcgggttcctttccccagcagtgggcacactgttccggttcccaggaggggtgtctggcgaggagtcccaccactcggagtccagggccagacagtgtggccttgactcgagaggcctcttggtccggagccctgtttccaagagtgcagcagcccctactgtgacctctgtgagaggaacctcggcgcactttgggattcagctcagaggtggcaccagattgcctgacaggcttagctggccgtgtggccctgggagtgctgggtggcagcaagagtttgcagccatggatagttctgagaccctggacgccagctgggaggcagcctgcagcgatggagcaaggcgtgtccgggcagcaggctctctgccatcagcagagttgagtagcaacagctgcagccctggctgtggccctgaggtccccccaacccctcctggctctcacagtgcctttacctcaagctttagctttattcggctctcgcttggctctgccggggaacgtggagaagcagaaggctgcccaccatccagagaggctgagtcccattgccagagcccccaggagatgggagccaaagctgccagcttggacgggcctcacgaggacccgcgatgtctctctcagcccttcagtctcttggctacacgggtctctgcagacttggcccaggccgcaaggaacagctccaggccagagcgtgacatgcattctttaccagacatggaccctggctcctccagttctctggatccctcactggctggctgtggtggtgatgggagcagcggctcaggggatgcccactcttgggacaccctgctcaggaaatgggagccagtgctgcgggactgcctgctgagaaaccggaggcagatggaggtaatatccttaagattaaaacttcagaaacttcaggaagatgcagttgagaatgatgattatgataaagctgagacgttacaacaaagattagaagacctggaacaagagaaaatcagcctgcactttcaacttccttcaaggcagccagctcttagcagtttcctgggtcacctggcagcacaagtccaggctgccttgcgccgtggggccactcagcaggccagcggagatgacacccacaccccactgagaatggagccgaggctgttggaacccactgctcaggacagcttgcacgtgtccatcacgagacgagactggcttcttcaggaaaagcagcagctacagaaagaaatcgaagctctccaagcaaggatgtttgtgctggaagccaaagatcaacagctgagaagggaaatagaggagcaagagcagcaactccagtggcagggctgcgacctgaccccactggtgggccagctgtccctgggtcagctgcaggaggtcagcaaggccttgcaggacaccctggcctcagccggtcagattcccttccatgcagagccaccggaaaccataaggagcctccaggaaagaataaaatccctcaacttgtcacttaaagaaatcactactaaggtgtgtatgagtgagaaattctgcagcaccctgaggaagaaagttaacgatattgaaacccaactaccagccttgcttgaagccaaaatgcatgccatatcaggaaaccatttctggacggctaaagacctcaccgaggagattagatcattaacatcagagagagaagggctggagggactcctcagcaagctgttggtgttgagttccaggaatgtcaaaaagctgggaagtgttaaagaagattacaacagactgagaagagaagtggagcaccaggagactgcctatgaaacaagtgtgaaggaaaatactatgaagtacatggaaacacttaagaataaactgtgcagctgcaagtgtccactgcttgggaaagtgtgggaagctgacttggaagcttgtcgattgcttatccagtgcctacagctccaggaagccaggggaagcctgtctgtagaagatgagaggcagatggatgacttagagggagctgctcctcctattccccccaggctccactccgaggataaaaggaagacccctttgaaggtattggaagaatggaagactcacctcatcccctctctgcactgtgctggaggtgaacagaaagaggaatcttacatcctttctgcagaacttggagaaaagtgtgaagacataggcaagaagctattgtacttggaagatcaacttcacacagcaatccacagtcatgatgaagatctcattcagtctctcaggagggagctccagatggtgaaggaaactctgcaggccatgatcctgcagctccagccagcaaaggaggcgggagaaagagaagctgcagcttcctgcatgacagctggtgtccacgaagcacaagcctacccatacgacgtcccagactacgct

SEQ ID NO: 16 - protein, 863 aa, Artificial
  Synthetic Construct
MPGGGPQGAPAAAGGGGVSHRAGSRDCLPPAACFRRRRLARRPGYMRSSTGPGIGFLSPAVGTLFRFPGGVSGEESHHSESRARQCGLDSRGLLVRSPVSKSAAAPTVTSVRGTSAHFGIQLRGGTRLPDRLSWPCGPGSAGWQQEFAAMDSSETLDASWEAACSDGARRVRAAGSLPSAELSSNSCSPGCGPEVPPTPPGSHSAFTSSFSFIRLSLGSAGERGEAEGCPPSREAESHCQSPQEMGAKAASLDGPHEDPRCLSQPFSLLATRVSADLAQAARNSSRPERDMHSLPDMDPGSSSSLDPSLAGCGGDGSSGSGDAHSWDTLLRKWEPVLRDCLLRNRRQMEVISLRLKLQKLQEDAVENDDYDKAETLQQRLEDLEQEKISLHFQLPSRQPALSSFLGHLAAQVQAALRRGATQQASGDDTHTPLRMEPRLLEPTAQDSLHVSITRRDWLLQEKQQLQKEIEALQARMFVLEAKDQQLRREIEEQEQQLQWQGCDLTPLVGQLSLGQLQEVSKALQDTLASAGQIPFHAEPPETIRSLQERIKSLNLSLKEITTKVCMSEKFCSTLRKKVNDIETQLPALLEAKMHAISGNHFWTAKDLTEEIRSLTSEREGLEGLLSKLLVLSSRNVKKLGSVKEDYNRLRREVEHQETAYETSVKENTMKYMETLKNKLCSCKCPLLGKVWEADLEACRLLIQCLQLQEARGSLSVEDERQMDDLEGAAPPIPPRLHSEDKRKTPLKVLEEWKTHLIPSLHCAGGEQKEESYILSAELGEKCEDIGKKLLYLEDQLHTAIHSHDEDLIQSLRRELQMVKETLQAMILQLQPAKEAGEREAAASCMTAGVHEAQAYPYDVPDYA

SEQ ID NO: 17 - DNA, 1941 bp, Artificial
  Synthetic KIAA-GFP
atgcaacagaaggcttttgaggaaagcagatatccctggcaggagtcctttgagaatgttgctgtgtgcctgccattacgctgcccgaggtgtggagaccataccagatttagaagcttgtcatccttgagggcccatctggagttcagtcacagctacgaagaaagaaccctcttgacaaaatgcagtctctttccatccctcaaagacacagacctagtcacttcctcagaactcctgaaaccgggaaaattgcagagcagtggcaacgtggtaaagcagaaaccgagctatgttaacttgtacagcatttcacatgaacattccaaggacaggaagccatttgaggtggtggcagagaggcctgtgtcctatgtgcagacctacactgccatggacctccatgcagactcgctggatgggacacggtcgggtcctggactgcccacctcagacaccaaagcttctttcgaggcacatgtcagagaaaaattcaatcgaatggttgaggctgtggataggaccattgagaagagaattgataaactcaccaaagagttggcccagaaaactgcggaactgttggaagttcgggcagcttttgtgcagctgactcagaaaaagcaggaagttcagagacgagagcgggccttaaacagacaggtggacgtggccgtggaaatgatagctgtactgaggcaacgcctgacggaatctgaggaggagcttcttaggaaagaagaagaagttgtcacattcaaccatttcctggaagcggcagctgagaaggaggttcaagggaaagcccggctccaggactttattgagaatctgttacagcgggtagaactggcggagaagcagcttgagtactatcagagccagcaggcctctggctttgtccgtgatctcagcgggcacgtgcttacagacatctcctcaaataggaagcccaaatgcctaagccgagggcacccgcattcggtatgtaaccaccctgatctcaagtcccatttccacccaaagggaaggaaccacctgaaaaaggccaaggatgacagagccagcatgcagcctgccaaggccattcacgaacaggctgagtcctcaagagacctctgcagacctccaaagaaaggggagctcctggggtttggccgcaaaggcaacatcaggcccaaaatggctaaaaaaaagccaacagccattgtgaacatcatcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 18 - protein, 646 aa, Artificial [sequence omitted]

SEQ ID NO: 19 - DNA, 554 bp, Artificial
  Synthetic Construct
ccatttgaggtggtggcagagaggcctgtgtcctatgtgcagacctacactgccatggacctccatgcagactcgctggatgggacacggtcgggtcctggactgcccacctcagacaccaaagcttctttcgaggcacatgtcagagaaaaattcaatcgaatggttgaggctgtggataggaccattgagaagagaattgataaactcaccaaagagttggcccagaaaactgcggaactgttggaagttcgggcagcttttgtgcagctgactcagaaaaagcaggaagttcagagacgagagcgggccttaaacagacaggtggacgtggccgtggaaatgatagctgtactgaggcaacgcctgacggaatctgaggaggagcttcttaggaaagaagaagaagttgtcacattcaaccatttcctggaagcggcagctgagaaggaggttcaagggaaagcccggctccaggactttattgagaatctgttacagcgggtagaactggcggagaagcagcttgagtactatcagagccagcaggcctc

(19) United States
(12) Patent Application Publication
(10) Pub. No.: US 2008/0107600 A1
(43) Pub. Date: May 8, 2008

(54) Gene Marker And Composition For Diagnosis And Treatment Of Neurological Disorders And Diseases And Use Of The Same

(76) Inventors: Taiichi Katayama, Osaka (JP); Takeshi Miyoshi, Okayama (JP); Masaya Tohyama, Osaka (JP); Kousuke Baba, Hyogo (JP); Akiko Honda, Hyogo (JP)

Correspondence Address: COZEN O'CONNOR, P.C., 1900 MARKET STREET, PHILADELPHIA, PA, 19103-3508, US

(21) Appl. No.: 10/545,564

(22) PCT Filed: Feb. 12, 2004
(86) PCT No.: PCT/JP04/01518

§ 371 Date: May 15, 2006

(30) Foreign Application Priority Data
Feb. 13, 2003 (JP) 2003-035787

(51) Int. Cl. A61K 45/00; C07K 2/00; C07H 21/00; C07K 14/00; A61P 25/18; A61P 25/00; G01N 33/566; C12Q 1/68; G01N 33/68

(52) U.S. Cl. 424/9.1; 530/300; 536/23.1; 536/24.33; 536/24.3; 530/350; 436/501; 435/6; 435/7.1; 514/789

(57) ABSTRACT

The present invention provides a marker, kit and method for determining the level of axon outgrowth and/or fasciculation, or conditions, disorders or diseases associated with the level of axon outgrowth and/or fasciculation. The present invention was achieved by unexpectedly finding the relationship between DISC1 and FEZ1 and KIAA0844 and unexpectedly finding that axon outgrowth and/or fasciculation are not normally conducted if the normal binding therebetween was inhibited. Therefore, the present invention relates to an agent specifically interacting with DISC1 and a gene product thereof, an agent specifically binding FEZ1 and a gene product thereof, an agent specifically binding FEZ1 and a gene product thereof, and an agent specifically binding KIAA0844 and a gene product thereof.

### TECHNICAL FIELD

The present invention relates to a method, marker, and kit for diagnosis and detection of conditions, disorders, or diseases associated with the level of axon outgrowth and/or fasciculation. The present invention also relates to a method of utilizing interaction between DISC1 and/or FEZ1 and/or a molecule known as KIAA0844 (hereinafter also simply referred to as KIAA0844) to identify an agent associated with conditions, disorders, or diseases associated with the level of axon outgrowth and/or fasciculation.

### BACKGROUND ART

Schizophrenia is a debilitating mental disease that affects about 1% of the population. Like many other psychiatric disorders, schizophrenia is thought to involve the combined effects of multiple genetic components (e.g., McGuffin P., Owen M. J., Farmer A. E., Lancet, 1995, 346: 678-682; and Riley B., Williamson R., Nat. Med., 2000, 6: 253-255). Research such as linkage analyses and association studies have not yet identified definitive genes responsible for the disease (e.g., Sawa A., Snyder S. H., Science, 2002, 296: 692-695; Karayiorgou M., Gogos J. A., Neuron, 1997, 19: 967-979; Riley. B. P., McGuffin P., Am. J. Med. Genet., 2000, 97: 23-44; Berrettini W. H., Biol. Psychiatry, 2000, 48:531-538; Brzustowicz L. M., Hodgkinson K. A., Chow E. W. C., Honer W. G., Bassett A. S., Science, 2000, 288: 678-682; Straub R. E., MacLean C. J., O'Neill F. A., Burke J., Murphy B., Duke F. et al., Nature Genet., 1995, 11: 287-293; Blouin J. L., Dombroski B. A., Nath S. K., Lasseter V. K., Wolyniec P. S., Nestadt G. et al., Nature Genet., 1998, 20: 70-73; Williams J., Spurlock G., McGuffin P., Mallet J., Nothen M. M., Gill M. et al., Lancet 1996, 347: 1294-1296; Sklar P., Schwab S. G., Williams N. M., Daly M., Schaffner S., Maier W. et al., Nat. Genet., 2001, 28: 126-128; and Anney R. J., Rees M. I., Bryan E., Spurlock G., Williams N., Norton N. et al., Mol. Psychiatry, 2002, 7: 493-502).

In a large Scottish family, a balanced (1; 11) (q42.1; q14.3) translocation that segregated with schizophrenia and affective disorders with a LOD score of 7.1 was found (e.g., Millar J. K., Wilson-Annan J. C., Anderson S., Christie S., Taylor M. S., Semple C. A. M. et al., Hum. Mol. Genet., 2000, 9: 1415-1423: and Blackwood D. H. R., Fordyce A., Walker M. T., St. Clair D. M., Porteous D. J., Muir W. J., Am. J. Hum. Genet., 2001, 69: 428-433). DISC1 on chromosome 1 was identified as a novel gene disrupted by this translocation (Millar J. K., Wilson-Annan J. C., Anderson S., Christie S., Taylor M. S., Semple C. A. M. et al., Hum. Mol. Genet., 2000, 9: 1415-1423). Family members exhibited no distinctive features by which the psychiatric phenotype could be distinguished from unrelated cases (e.g., Blackwood D. H. R., Fordyce A., Walker M. T., St. Clair D. M., Porteous D. J., Muir W. J., Am. J. Hum. Genet., 2001, 69: 428-433). Moreover, translocation carriers showed a significant reduction in the amplitude of the P300 event-related potential, which was also observed in unrelated patients with schizophrenia (e.g., Blackwood D. H. R., Fordyce A., Walker M. T., St. Clair D. M., Porteous D. J., Muir W. J., Am. J. Hum. Genet., 2001, 69: 428-433). These findings suggest that disruption of the function of this gene may confer susceptibility to these mental disorders. A linkage report also indicated 1q42 as a possible locus for schizophrenia in a study of Finnish families (e.g., Ekelund J., Hovatta I., Parker A., Paunio T., Varilo T., Martin R. et al., Hum. Mol. Genet., 2001, 10: 1611-1617).

FEZ1 is a mammalian homologue of the Caenorhabditis elegans UNC-76 protein involved in axonal outgrowth and fasciculation. FEZ1 is reported to be involved in axonal outgrowth and fasciculation (e.g., Bloom L., Horvitz H. R., Proc. Natl. Acad. Sci. USA, 1997, 94: 3414-3419; and Kuroda S., Nakagawa N., Tokunaga C., Tatematsu K., Tanizawa K., J. Cell Biol., 1999, 144: 403-411).

Recent studies have provided reliable evidence that schizophrenia is a neurodevelopmental disorder (e.g., Sawa A., Snyder S. H., Science, 2002, 296: 692-695; Weinberger D. R., Arch. Gen. Psychiatry, 1987, 44: 660-669; and Lewis D. A., Levitt P., Annu. Rev. Neurosci., 2002, 25: 409-432). Cytoarchitectual changes in the hippocampus have been noteworthy among the various neuropathological abnormalities reported in schizophrenia (e.g., Harrison P. J., Brain, 1999, 122: 593-624; Heckers S., Konradi C., J. Neural. Transm., 2002, 109: 891-905; and Harrison P. J., Eastwood S. L., Hippocampus, 2001, 11: 508-519). Decreased neuronal size and alterations in presynaptic and dendritic markers suggest abnormalities in the hippocampal neural circuitry in schizophrenia (e.g., Harrison P. J., Eastwood S. L., Hippocampus, 2001, 11: 508-519).

A recent study has reported the existence of two forms of DISC1 in rat brain and the up-regulated expression of the full-length form at developing stages, suggesting the importance of the full-length form with respect to the development of the nervous system (e.g., Ozeki Y., Tomoda T., Kleiderlein J., Kamiya A., Bord L., Fujii K. et al., Proc. Natl. Acad. Sci., January 2003).

The possible implications of DISC1 in the cytoskeleton are also predicted by the recent study (e.g., Ozeki Y., Tomoda T., Kleiderlein J., Kamiya A., Bord L., Fujii K. et al., Proc. Natl. Acad. Sci., January 2003) and preliminary reports (e.g., Millar J. K., James R., Christie S., Taylor M. S., Devon R. S., Hogg G. et al., Abstract from Xth World Congress on Psychiatric Genetics, 2002; and Kandpal G., Ma L., Acton P., Austin C. P., Morris J. A., Abstract from Society for Neuroscience 32nd Annual Meeting, 2002).

There are signaling proteins, such as Rho GTPases, which orchestrate coordinated changes in the actin cytoskeleton essential for directed neurite outgrowth (e.g., Hall A., Science, 1998, 279: 509-514; and Luo L., Nature Rev. Neurosci., 2000, 1:173-180). Interestingly, the regulation of the actin cytoskeleton in the developing nervous system is involved in the pathogenesis of mental retardation (e.g., Billuart P., Bienvenu T., Ronce N., des Portes V., Vinet M. C., Zemni R. et al., Nature, 1998, 392: 923-926). Seven genes have been identified to be responsible for X-linked mental retardation when mutated (e.g., Chelly J., Mandel J. L., Nat. Rev. Genet., 2001, 2: 669-680). Three of these genes encode oligophrenin-1 (Billuart P., Bienvenu T., Ronce N., des Portes V., Vinet M. C., Zemni R. et al., Nature, 1998, 392: 923-926), PAK3 (Allen K. M., Gleeson J. G., Bagrodia S., Partington M. W., MacMillan J. C., Cerione R. A. et al., Nat. Genet., 1998, 20:25-30), and αPIX (Kutsche K., Yntema H., Brandt A., Jantke I., Nothwang H. G., Orth U. et al., Nat. Genet., 2000, 26: 247-250). These proteins interact directly with Rho GTPases.

The sequences of human genes have been determined from various libraries by Kazusa DNA Research Institute. However, substantially no functions of the genes have been clarified. KIAA0844 has been sequenced but its function has not yet been clarified (Nagase T. et al., DNA Res., 1998, 5(6):355-64).

In spite of the above-described research, a gene which is responsible for psychiatric diseases, such as schizophrenia or can be used as a diagnosis marker has yet to be identified. Therefore, there is a keen desire for identification of such a marker gene in the art.

There is also a keen desire for identification of a system for identifying an agent for treatment or prophylaxis of psychiatric diseases, such as schizophrenia, for which a crucial pharmaceutical agent is not available at present.

Hereinafter, the present invention will be described by way of preferred embodiments. It will be understood by those skilled in the art that the embodiments of the present invention can be appropriately made or carried out based on the description of the present specification and commonly used techniques well known in the art. The function and effect of the present invention can be easily recognized by those skilled in the art.

### DISCLOSURE OF THE INVENTION

The present inventors unexpectedly found that if the binding of DISC1 and FEZ1 and KIAA0844 is inhibited, axon outgrowth and/or fasciculation does not proceed normally. Based on this finding, the present invention provides a marker, kit, and method for determining the level of axon outgrowth and/or fasciculation, or conditions, disorders or diseases (e.g., schizophrenia) associated with such a level.

Particularly, neurological diseases have not yet fully analyzed and revealed at the molecular level in spite of the development of molecular biology and cellular biology. It has been suggested a gene called DISC1 is involved in schizophrenia, which is a neurological disease. To date the disruption of DISC1 by translocation has been found in a Scottish family, though almost no function or role of DISC1 have been revealed. In the present invention, the pathophysiological role of DISC1 was studied based on analysis at the molecular level and the organism level. As a result, it was found that the pathophysiological role of DISC1 and FEZ1 in neurological diseases (e.g., schizophrenia) was revealed by clarifying the close association of DISC1 with FEZ1 and the relationship between the binding of DISC1 with FEZ1 and axon outgrowth and/or fasciculation.

In the present invention, the relationship between KIAA0844 and axon outgrowth and/or fasciculation was also discovered by unexpectedly finding that KIAA0844 is closely associated with DISC1 and/or FEZ1.

Therefore, the present invention provides the following.

(1) An agent specifically interacting with:

(a) a polynucleotide having a base sequence set forth in SEQ ID NO. 1 or a fragment thereof;

(b) a polynucleotide encoding a polypeptide consisting of an amino acid sequence set forth in SEQ ID NO. 2 or a fragment thereof;

(c) a polynucleotide encoding a variant polypeptide having an amino acid sequence set forth in SEQ ID NO. 2 or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 1;

(e) a polynucleotide encoding a species homolog of a polypeptide consisting of an amino acid sequence set forth in SEQ ID NO. 2;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity.

### (2) An agent according to item 1, wherein the agent is selected from the group consisting of a nucleic acid molecule, a polypeptide, a lipid, a sugar chain, a low molecular weight organic molecule, and a composite molecule thereof.

### (3) An agent according to item 1, wherein the agent is a nucleic acid molecule of at least 8 contiguous nucleotides in length.

### (4) An agent according to item 1, wherein the agent is a nucleic acid molecule having a sequence having at least 70% identity to the nucleic acid sequence of any one of the polynucleotides of (a) to (g).

### (5) An agent according to item 1, wherein the agent is a nucleic acid molecule hybridizable to the nucleic acid sequence of any one of the polynucleotides of (a) to (g) under stringent conditions.

### (6) An agent according to item 3, wherein the agent is used as a primer.

### (7) An agent according to item 3, wherein the agent is used as a probe.

### (8) An agent according to item 1, wherein the polynucleotide or the polypeptide comprises a range encoding nucleotides 1095 to 2615 of SEQ ID NO. 1 or a range of amino acids 348 to 854 of SEQ ID NO. 2.

(9) An agent according to item 1, wherein the polynucleotide or the polypeptide comprises a range encoding a range selected from the group consisting of nucleotides 1095 to 1844, nucleotides 1845 to 2615, nucleotides 1095 to 1952, nucleotides 1095 to 1652, nucleotides 1653 to 1952, nucleotides 1391 to 1652, and nucleotides 1391 to 1952 of SEQ ID NO. 1, or a range selected from the group consisting of amino acids 348 to 597, amino acids 598 to 854, amino acids 348 to 633, amino acids 348 to 533, amino acids 534 to 633, amino acids 446 to 533, and amino acids 446 to 633 of SEQ ID NO. 2.

### (10) An agent according to item 1, wherein the agent is labeled or labelable.

(11) An agent specifically interacting with a polypeptide, wherein the polypeptide comprises:

(a) a polypeptide consisting of an amino acid sequence set forth in SEQ ID NO. 2 or a fragment thereof;

(b) a polypeptide having an amino acid sequence set forth in SEQ ID NO. 2 or a fragment thereof, wherein at least one amino acid in the sequence has a mutation selected from the group consisting of substitution, addition, and deletion and wherein the polypeptide has biological activity;

(c) a polypeptide encoded by a spliced mutant or alleic mutant of a base sequence set forth in SEQ ID NO. 1;

(d) a polypeptide being a species homolog of an amino acid sequence set forth in SEQ ID NO. 2; or

(e) a polypeptide having an amino acid sequence having at least 70% identity to any one of the polypeptides (a) to (d), and having biological activity.

### (12) An agent according to item 11, wherein the agent is selected from the group consisting of a nucleic acid molecule, a polypeptide, a lipid, a sugar chain, a low molecular weight organic molecule, and a composite molecule thereof.

### (13) An agent according to item 11, wherein the agent is an agent or a derivative thereof.

### (14) An agent according to item 11, wherein the agent is used as a probe.

### (15) An agent according to item 11, wherein the polypeptide comprises a range of amino acids 348 to 854 of SEQ ID NO. 2.

(16) An agent according to item 11, wherein the polypeptide comprises a range selected from the group consisting of amino acids 348 to 597, amino acids 598 to 854, amino acids 348 to 633, amino acids 348 to 533, amino acids 534 to 633, amino acids 446 to 533, and amino acids 446 to 633 of SEQ ID NO. 2.

### (17) An agent according to item 11, wherein the agent is labeled or labelable.

(18) An agent specifically interacting with:

(a) a polynucleotide having a base sequence set forth in SEQ ID NO. 3 or a fragment thereof;

(b) a polynucleotide encoding a polypeptide consisting of an amino acid sequence set forth in SEQ ID NO. 4 or a fragment thereof;

(c) a polynucleotide encoding a variant polypeptide having an amino acid sequence set forth in SEQ ID NO. 4 or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 3;

(e) a polynucleotide encoding a species homolog of a polypeptide consisting of an amino acid sequence set forth in SEQ ID NO. 4;

(f) a polynucleotide hybridizable to any one of the polynucleotides of (a) to (e) under stringent conditions and encoding a polypeptide having biological activity; or

(g) a polynucleotide consisting of a base sequence having at least 70. % identity to any one of the polynucleotides (a) to (e) or a complementary sequence thereof, and encoding a polypeptide having biological activity.

### (19) An agent according to item 18, wherein the agent is selected from the group consisting of a nucleic acid molecule, a polypeptide, a lipid, a sugar chain, a low molecular weight organic molecule, and a composite molecule thereof.

### (20) An agent according to item 18, wherein the agent is a nucleic acid molecule of at least 8 contiguous nucleotides in length.

### (21) An agent according to item 18, wherein the agent is a nucleic acid molecule having a sequence having at least 70% identity to the nucleic acid sequence of any one of the polynucleotides of (a) to (g).

### (22) An agent according to item 18, wherein the agent is a nucleic acid molecule hybridizable to the nucleic acid sequence of any one of the polynucleotides of (a) to (g) under stringent conditions.

### (23) An agent according to item 20, wherein the agent is used as a primer.

### (24) An agent according to item 20, wherein the agent is used as a probe.

### (25) An agent according to item 18, wherein the polynucleotide or the polypeptide comprises a range encoding nucleotides 478 to 1269 of SEQ ID NO. 3 or a range of amino acids 129 to 392 of SEQ ID NO. 4.

### (26) An agent according to item 18, wherein the polynucleotide or the polypeptide comprises a range encoding nucleotides 832 to 1269 of SEQ ID NO. 3 or a range of amino acids 247 to 392 of SEQ ID NO. 4.

### (27) An agent according to item 18, wherein the agent is labeled or labelable.

### (28) An agent specifically interacting with a polypeptide,

wherein the polypeptide comprises:

(a) a polypeptide consisting of an amino acid sequence set forth in SEQ ID NO. 4 or a fragment thereof;

(b) a polypeptide having an amino acid sequence set forth in SEQ ID NO. 4 or a fragment thereof, wherein at least one amino acid in the sequence has a mutation selected from the group consisting of substitution, addition, and deletion and wherein the polypeptide has biological activity;

(c) a polypeptide encoded by a spliced mutant or alleic mutant of a base sequence set forth in SEQ ID NO. 3;

(d) a polypeptide being a species homolog of an amino acid sequence set forth in SEQ ID NO. 4; or

(e) a polypeptide having an amino acid sequence having at least 70% identity to any one of the polypeptides (a) to (d), and having biological activity.

### (29) An agent according to item 28, wherein the agent is selected from the group consisting of a nucleic acid molecule, a polypeptide, a lipid, a sugar chain, a low molecular weight organic molecule, and a composite molecule thereof.

### (30) An agent according to item 28, wherein the agent is an agent or a derivative thereof.

### (31) An agent according to item 28, wherein the agent is used as a probe.

### (32) An agent according to item 28, wherein the polypeptide comprises a range of amino acids 129 to 392 of SEQ ID NO. 4.

### (33) An agent according to item 28, wherein the polypeptide comprises a range of amino acids 247 to 392 of SEQ ID NO. 4.

### (34) An agent according to item 28, wherein the agent is labeled or labelable.

(35) A composition for determining a function of FEZ1 or KIAA0844, comprising:

(A) an agent specifically interacting with:

(a) a polynucleotide having a base sequence set forth in SEQ ID NO. 1 or a fragment thereof;

(b) a polynucleotide encoding a polypeptide consisting of an amino acid sequence set forth in SEQ ID NO. 2 or a fragment thereof;

(c) a polynucleotide encoding a variant polypeptide having an amino acid sequence set forth in SEQ ID NO. 2 or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 1;

(e) a polynucleotide encoding a species homolog of a polypeptide consisting of an amino acid sequence set forth in SEQ ID NO. 2;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity; and/or

(B) an agent specifically interacting with a polypeptide, wherein the polypeptide comprises:

(a) a polypeptide consisting of an amino acid sequence set forth in SEQ ID NO. 2 or a fragment thereof;

(b) a polypeptide having an amino acid sequence set forth in SEQ ID NO. 2 or a fragment thereof, wherein at least one amino acid in the sequence has a mutation selected from the group consisting of substitution, addition, and deletion and wherein the polypeptide has biological activity;

(c) a polypeptide encoded by a spliced mutant or alleic mutant of a base sequence set forth in SEQ ID NO. 1;

(d) a polypeptide being a species homolog of an amino acid sequence set forth in SEQ ID NO. 2; or

(e) a polypeptide having an amino acid sequence having at least 70% identity to any one of the polypeptides (a) to (d), and having biological activity.

(36) A composition for determining a function of DISC1 or KIAA0844, comprising:

(A) an agent specifically interacting with:

(a) a polynucleotide having a base sequence set forth in SEQ ID NO. 3 or a fragment thereof;

(b) a polynucleotide encoding a polypeptide consisting of an amino acid sequence set forth in SEQ ID NO. 4 or a fragment thereof;

(c) a polynucleotide encoding a variant polypeptide having an amino acid sequence set forth in SEQ ID NO. 4 or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 3;

(e) a polynucleotide encoding a species homolog of a polypeptide consisting of an amino acid sequence set forth in SEQ ID NO. 4;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity; and/or

(B) an agent specifically interacting with a polypeptide, wherein the polypeptide comprises:

(a) a polypeptide consisting of an amino acid sequence set forth in SEQ ID NO. 4 or a fragment thereof;

(b) a polypeptide having an amino acid sequence set forth in SEQ ID NO. 4 or a fragment thereof, wherein at least one amino acid in the sequence has a mutation selected from the group consisting of substitution, addition, and deletion and wherein the polypeptide has biological activity;

(c) a polypeptide encoded by a spliced mutant or alleic mutant of a base sequence set forth in SEQ ID NO. 3;

(d) a polypeptide being a species homolog of an amino acid sequence set forth in SEQ ID NO. 4; or

(e) a polypeptide having an amino acid sequence having at least 70% identity to any one of the polypeptides (a) to (d), and having biological activity.

(37) A composition for determining a level of axon outgrowth and/or fasciculation, or a condition, disorder or disease associated with the level of axon outgrowth and/or fasciculation, comprising:

(A) an agent specifically interacting with:

(a) a polynucleotide having a base sequence set forth in SEQ ID NO. 1 or a fragment thereof;

(b) a polynucleotide encoding a polypeptide consisting of an amino acid sequence set forth in SEQ ID NO. 2 or a fragment thereof;

(c) a polynucleotide encoding a variant polypeptide having an amino acid sequence set forth in SEQ ID NO. 2 or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 1;

(e) a polynucleotide encoding a species homolog of a polypeptide consisting of an amino acid sequence set forth in SEQ ID NO. 2;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity;

(B) an agent specifically interacting with a polypeptide, wherein the polypeptide comprises:

(a) a polypeptide consisting of an amino acid sequence set forth in SEQ ID NO. 2 or a fragment thereof;

(b) a polypeptide having an amino acid sequence set forth in SEQ ID NO. 2 or a fragment thereof, wherein at least one amino acid in the sequence has a mutation selected from the group consisting of substitution, addition, and deletion and wherein the polypeptide has biological activity;

(c) a polypeptide encoded by a spliced mutant or alleic mutant of a base sequence set forth in SEQ ID NO. 1;

(d) a polypeptide being a species homolog of an amino acid sequence set forth in SEQ ID NO. 2; or

(e) a polypeptide having an amino acid sequence having at least 70% identity to any one of the polypeptides (a) to (d), and having biological activity;

(C) an agent specifically interacting with:

(a) a polynucleotide having a base sequence set forth in SEQ ID NO. 3 or a fragment thereof;

(b) a polynucleotide encoding a polypeptide consisting of an amino acid sequence set forth in SEQ ID NO. 4 or a fragment thereof:

(c) a polynucleotide encoding a variant polypeptide having an amino acid sequence set forth in SEQ ID NO. 4 or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 3;

(e) a polynucleotide encoding a species homolog of a polypeptide consisting of an amino acid sequence set forth in SEQ ID NO. 4;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity; and/or

(D) an agent specifically interacting with a polypeptide, wherein the polypeptide comprises:

(a) a polypeptide consisting of an amino acid sequence set forth in SEQ ID NO. 4 or a fragment thereof;

(b) a polypeptide having an amino acid sequence set forth in SEQ ID NO. 4 or a fragment thereof, wherein at least one amino acid in the sequence has a mutation selected from the group consisting of substitution, addition, and deletion and wherein the polypeptide has biological activity;

(c) a polypeptide encoded by a spliced mutant or alleic mutant of a base sequence set forth in SEQ ID NO. 3;

(d) a polypeptide being a species homolog of an amino acid sequence set forth in SEQ ID NO. 4; or

(e) a polypeptide having an amino acid sequence having at least 70% identity to any one of the polypeptides (a) to (d), and having biological activity.

### (38) A composition according to item 37, wherein the condition, disorder or disease associated with the level of axon outgrowth and/or fasciculation is schizophrenia or mental retardation.

(39) A method for diagnosis of a condition, disorder or disease associated with a level of axon outgrowth and/or fasciculation, comprising the steps of:

(a) measuring binding of DISC1 and FEZ1 in a sample derived from a subject; and

(b) comparing a level of the measured binding with a level of normal binding, wherein when the level of the measured binding is lower than the level of the normal binding, the level of axon outgrowth and/or fasciculation is inferior to a normal level thereof.

### (40) A method according to item 39, wherein the condition, disorder or disease associated with the level of axon outgrowth and/or fasciculation is schizophrenia or mental retardation.

(41) A method for diagnosis of a condition, disorder or disease associated with a level of axon outgrowth and/or fasciculation, comprising the steps of:

(a) measuring binding of DISC1 and FEZ1 in a subject in need of diagnosis of a condition, disorder or disease associated with a level of axon outgrowth and/or fasciculation;

(b) measuring binding of DISC1 and FEZ1 in a normal subject: and

(c) comparing a level of the binding of (a) with a level of the binding of (b),

wherein when the level of the binding of (a) is higher or lower than the level of the binding of (b), the subject is diagnosed as having an abnormality, disorder or disease associated with a level of axon outgrowth and/or fasciculation.

### (42) A method according to item 41, wherein the condition, disorder or disease associated with the level of axon outgrowth and/or fasciculation is schizophrenia or mental retardation.

### (43) A method according to item 41, wherein in the steps of (a) and (b), an antibody against a first polypeptide having an amino acid sequence having at least 70% homology to a sequence set forth in SEQ ID NO. 2, or a fragment thereof, is used.

### (44) A method according to item 41, wherein in the steps of (a) and (b), an antibody against a second polypeptide having an amino acid sequence having at least 70% homology to a sequence set forth in SEQ ID NO. 4, or a fragment thereof, is used.

(45) A method according to item 41, wherein in the steps of (a) and (b), an antibody against a first polypeptide having an amino acid sequence having at least 70% homology to a sequence set forth in SEQ ID NO. 2, or a fragment thereof, and an antibody against a second polypeptide having an amino acid sequence having at least 70% homology to a sequence set forth in SEQ ID NO. 4, or a fragment thereof, are used.
(46) A method for detecting in a genetic mutation associated with a condition, disorder or disease associated with a level of axon outgrowth and/or fasciculation, comprising the step of:

detecting in a mutation in a polynucleotide sequence of a DISC1 gene and/or a FEZ1 gene in a sample.

### (47) A method according to item 46, wherein the condition, disorder or disease associated with the level of axon outgrowth and/or fasciculation is schizophrenia or mental retardation.

### (48) A method according to item 46, wherein the mutation is linked with a condition, disorder or disease associated with a level of axon outgrowth and/or fasciculation.

(49) A kit for diagnosis of a condition, disorder or disease associated with a level of axon outgrowth and/or fasciculation, comprising:

(a) a composition according to item 37; and

(b) instructions,

the instructions describes:
(i) using the composition to measure binding of DISC1 and FEZ1 in a subject in need of diagnosis of the condition, disorder or disease associated with the level of axon outgrowth and/or fasciculation;
(ii) using the composition to measure binding of DISC1 and FEZ1 in a normal subject; and
(iii) comparing a level of the binding of (i) with a level of the binding of (ii),
wherein when the level of the binding of (i) is higher or lower than the level of the binding of (ii), the subject is diagnosed as having an abnormality, disorder or disease associated with a level of axon outgrowth and/or fasciculation.

### (50) A kit according to item 49, wherein the condition, disorder or disease associated with the level of axon outgrowth and/or fasciculation is schizophrenia or mental retardation.

(51) A kit for detection of a condition, disorder or disease associated with a level of axon outgrowth and/or fasciculation, comprising:

(a) at least 2 primers of at least 10 nucleotides in length, wherein each of the primers comprises:
(i) a polynucleotide having at least 70% homology to a polynucleotide set forth in SEQ ID NO. 1: and
(ii) a primer having at least 70% homology to a polynucleotide encoding an amino acid sequence set forth in SEQ ID NO. 2 and hybridizable to the polynucleotide at different positions in the amino acid sequence under stringent conditions; and

(b) instructions describing using the primer of (a) to detect a nucleic acid sequence in a sample derived from the subject and detect a mutation in the polynucleotide set forth in SEQ ID NO. 1.

### (52) A kit according to item 51, wherein the detected nucleic acid sequence is nucleotides 54 to 2615 in SEQ ID NO. 1.

(53) A kit for detection of a condition, disorder or disease associated with a level of axon outgrowth and/or fasciculation, comprising:

(a) at least 2 primers of at least 10 nucleotides in length, wherein each of the primers comprises:
(i) a polynucleotide having at least 70% homology to a polynucleotide set forth in SEQ ID NO. 3; and
(ii) a primer having at least 70% homology to a polynucleotide encoding an amino acid sequence set forth in SEQ ID NO. 4 and hybridizable to the polynucleotide at different positions in the amino acid sequence under stringent conditions; and

(b) instructions describing using the primer of (a) to detect a nucleic acid sequence in a sample derived from the subject and detect a mutation in the polynucleotide set forth in SEQ ID NO. 3.

### (54) A kit according to item 53, wherein the detected nucleic acid sequence is nucleotides 94 to 1269 in SEQ ID NO. 3.

(55) A method for identifying an agent regulating a condition, disorder or disease associated with a level of axon outgrowth and/or fasciculation, comprising:

(a) contacting a first polypeptide having an amino acid sequence having at least 70% homology to SEQ ID NO. 2 or a fragment thereof with a second polypeptide having an amino acid sequence having at least 70% homology to SEQ ID NO. 4 or a fragment thereof in the presence of a test agent; and

(b) comparing a level of binding of the first polypeptide and the second polypeptide with a level of binding therebetween in the absence of the test agent,

wherein when the level of binding in the presence of the test agent is lower than the level of binding in the absence of the test agent, the test agent is a negative-regulatory agent for the condition, disorder or disease associated with the level of axon outgrowth and/or fasciculation; and when the level of binding in the presence of the test agent is higher than the level of binding in the absence of the test agent, the test agent is a positive-regulatory agent for the condition, disorder or disease associated with the level of axon outgrowth and/or fasciculation.

### (56) A method according to item 55, wherein the condition, disorder or disease associated with the level of axon outgrowth and/or fasciculation is schizophrenia or mental retardation.

### (57) A method according to item 55, wherein the first polypeptide comprises amino acids 446 to 597 in SEQ ID NO. 2.

### (58) A method according to item 55, wherein the second polypeptide comprises amino acids 247 to 392 in SEQ ID NO. 4.

### (59) A method according to item 57, wherein the second polypeptide comprises amino acids 247 to 392 in SEQ ID NO. 4.

### (60) A method according to item 55, wherein the step of (a) comprises contacting a cell expressing the first polypeptide with a cell expressing the second peptide.

### (61) A regulatory agent, identified by a method according to item 55.

### (62) A pharmaceutical composition, comprising a regulatory agent according to item 61.

(63) A method for treatment or prophylaxis of a condition, disorder or disease associated with a level of axon outgrowth and/or fasciculation, comprising the step of:

administrating a pharmaceutical composition according to item 62 into a subject.

### (64) A method according to item 63, wherein the condition, disorder or disease associated with the level of axon outgrowth and/or fasciculation is schizophrenia or mental retardation.

(65) An agent specifically interacting with:

(a) a polynucleotide having a base sequence set forth in SEQ ID NO. 13 or a fragment thereof;

(b) a polynucleotide encoding a polypeptide consisting of an amino acid sequence set forth in SEQ ID NO. 14 or a fragment thereof;

(c) a polynucleotide encoding a variant polypeptide having an amino acid sequence set forth in SEQ ID NO. 14 or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 13;

(e) a polynucleotide encoding a species homolog of a polypeptide consisting of an amino acid sequence set forth in SEQ ID NO. 14;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity.

### (66) An agent according to item 65, wherein the base sequence set forth in SEQ ID NO. 13 is provided as KIAA0844.

### (67) An agent according to item 65, wherein the agent is selected from the group consisting of a nucleic acid molecule, a polypeptide, a lipid, a sugar chain, a low molecular weight organic molecule, and a composite molecule thereof.

### (68) An agent according to item 65, wherein the agent is a nucleic acid molecule of at least 8 contiguous nucleotides in length.

### (69) An agent according to item 65, wherein the agent is a nucleic acid molecule having a sequence having at least 70% identity to the nucleic acid sequence of any one of the polynucleotides of (a) to (g).

### (70) An agent according to item 65, wherein the agent is a nucleic acid molecule hybridizable to the nucleic acid sequence of any one of the polynucleotides of (a) to (g) under stringent conditions.

### (71) An agent according to item 67, wherein the agent is used as a primer.

### (72) An agent according to item 67, wherein the agent is used as a probe.

### (73) An agent according to item 65, wherein the agent is labeled or labelable.

(74) An agent specifically interacting with a polypeptide, wherein the polypeptide comprises:

(a) a polypeptide consisting of an amino acid sequence set forth in SEQ ID NO. 14 or a fragment thereof;

(b) a polypeptide having an amino acid sequence set forth in SEQ ID NO. 14 or a fragment thereof, wherein at least one amino acid in the sequence has a mutation selected from the group consisting of substitution, addition, and deletion and wherein the polypeptide has biological activity;

(c) a polypeptide encoded by a spliced mutant or alleic mutant of a base sequence set forth in SEQ ID NO. 13;

(d) a polypeptide being a species homolog of an amino acid sequence set forth in SEQ ID NO. 14; or

(e) a polypeptide having an amino acid sequence having at least 70% identity to any one of the polypeptides (a) to (d), and having biological activity.

### (75) An agent according to item 74, wherein the base sequence set forth in SEQ ID NO. 13 is provided as KIAA0844.

### (76) An agent according to item 74, wherein the agent is selected from the group consisting of a nucleic acid molecule, a polypeptide, a lipid, a sugar chain, a low molecular weight organic molecule, and a composite molecule thereof.

### (77) An agent according to item 74, wherein the agent is an agent or a derivative thereof.

### (78) An agent according to item 74, wherein the agent is used as a probe.

### (79) An agent according to item 74, wherein the agent is labeled or labelable.

(80) A composition for determining a function of FEZ1, comprising:

(A) an agent specifically interacting with:

(a) a polynucleotide having a base sequence set forth in SEQ ID NO. 13 or a fragment thereof;

(b) a polynucleotide encoding a polypeptide consisting of an amino acid sequence set forth in SEQ ID NO. 14 or a fragment thereof;

(c) a polynucleotide encoding a variant polypeptide having an amino acid sequence set forth in SEQ ID NO. 14 or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 13;

(e) a polynucleotide encoding a species homolog of a polypeptide consisting of an amino acid sequence set forth in SEQ ID NO. 14;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity; and/or

(B) an agent specifically interacting with a polypeptide, wherein the polypeptide comprises:

(a) a polypeptide consisting of an amino acid sequence set forth in SEQ ID NO. 14 or a fragment thereof;

(b) a polypeptide having an amino acid sequence set forth in SEQ ID NO. 14 or a fragment thereof, wherein at least one amino acid in the sequence has a mutation selected from the group consisting of substitution, addition, and deletion and wherein the polypeptide has biological activity;

(c) a polypeptide encoded by a spliced mutant or alleic mutant of a base sequence set forth in SEQ ID NO. 13;

(d) a polypeptide being a species homolog of an amino acid sequence set forth in SEQ ID NO. 14; or

(e) a polypeptide having an amino acid sequence having at least 70% identity to any one of the polypeptides (a) to (d), and having biological activity.

(81) A composition for determining a function of DISC1, comprising:

(A) an agent specifically interacting with:

(a) a polynucleotide having a base sequence set forth in SEQ ID NO. 13 or a fragment thereof;

(b) a polynucleotide encoding a polypeptide consisting of an amino acid sequence set forth in SEQ ID NO. 14 or a fragment thereof;

(c) a polynucleotide encoding a variant polypeptide having an amino acid sequence set forth in SEQ ID NO. 14 or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 13;

(e) a polynucleotide encoding a species homolog of a polypeptide consisting of an amino acid sequence set forth in SEQ ID NO. 14;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity; and/or

(B) an agent specifically interacting with a polypeptide, wherein the polypeptide comprises:

(a) a polypeptide consisting of an amino acid sequence set forth in SEQ ID NO. 14 or a fragment thereof;

(b) a polypeptide having an amino acid sequence set forth in SEQ ID NO. 14 or a fragment thereof, wherein at least one amino acid in the sequence has a mutation selected from the group consisting of substitution, addition, and deletion and wherein the polypeptide has biological activity;

(c) a polypeptide encoded by a spliced mutant or alleic mutant of a base sequence set forth in SEQ ID NO. 13;

(d) a polypeptide being a species homolog of an amino acid sequence set forth in SEQ ID NO. 14; or

(e) a polypeptide having an amino acid sequence having at least 70% identity to any one of the polypeptides (a) to (d), and having biological activity.

(82) A composition for determining a level of axon outgrowth and/or fasciculation, or a condition, disorder or disease associated with the level of axon outgrowth and/or fasciculation, comprising:

(A) an agent specifically interacting with:

(a) a polynucleotide having a base sequence set forth in SEQ ID NO. 13 or a fragment thereof;

(b) a polynucleotide encoding a polypeptide consisting of an amino acid sequence set forth in SEQ ID NO. 14 or a fragment thereof;

(c) a polynucleotide encoding a variant polypeptide having an amino acid sequence set forth in SEQ ID NO. 14 or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 13;

(e) a polynucleotide encoding a species homolog of a polypeptide consisting of an amino acid sequence set forth in SEQ ID NO. 14;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity; and/or

(B) an agent specifically interacting with a polypeptide, wherein the polypeptide comprises:

(a) a polypeptide consisting of an amino acid sequence set forth in SEQ ID NO. 14 or a fragment thereof;

(b) a polypeptide having an amino acid sequence set forth in SEQ ID NO. 14 or a fragment thereof, wherein at least one amino acid in the sequence has a mutation selected from the group consisting of substitution, addition, and deletion and wherein the polypeptide has biological activity;

(c) a polypeptide encoded by a spliced mutant or alleic mutant of a base sequence set forth in SEQ ID NO. 13;

(d) a polypeptide being a species homolog of an amino acid sequence set forth in SEQ ID NO. 14; or

(e) a polypeptide having an amino acid sequence having at least 70% identity to any one of the polypeptides (a) to (d), and having biological activity.

### (83) A composition according to item 82, wherein the condition, disorder or disease associated with the level of axon outgrowth and/or fasciculation is schizophrenia or mental retardation.

(84) A method for diagnosis of a condition, disorder or disease associated with a level of axon outgrowth and/or fasciculation, comprising the steps of:

(a) measuring binding of DISC1 or FEZ1 and KIAA0844 in a sample derived from a subject; and

(b) comparing a level of the measured binding with a level of normal binding, wherein when the level of the measured binding is lower than the level of the normal binding, the level of axon outgrowth and/or fasciculation is inferior to a normal level thereof.

### (85) A method according to item 84, wherein the condition, disorder or disease associated with the level of axon outgrowth and/or fasciculation is schizophrenia or mental retardation.

(86) A method for diagnosis of a condition, disorder or disease associated with a level of axon outgrowth and/or fasciculation, comprising the steps of:

(a) measuring binding of DISC1 or FEZ1 and KIAA0844 in a subject in need of diagnosis of a condition, disorder or disease associated with a level of axon outgrowth and/or fasciculation:

(b) measuring binding of DISC1 or FEZ1 and KIAA0844 in a normal subject; and

(c) comparing a level of the binding of (a) with a level of the binding of (b),

wherein when the level of the binding of (a) is higher or lower than the level of the binding of (b), the subject is diagnosed as having an abnormality, disorder or disease associated with a level of axon outgrowth and/or fasciculation.

(87) A kit for diagnosis of a condition, disorder or disease associated with a level of axon outgrowth and/or fasciculation, comprising:

(a) a composition according to item 82; and

(b) instructions,

the instructions describes:
(i) using the composition to measure binding of DISC1 and FEZ1 in a subject in need of diagnosis of the condition, disorder or disease associated with the level of axon outgrowth and/or fasciculation;
(ii) using the composition to measure binding of DISC1 and FEZ1 in a normal subject; and
(iii) comparing a level of the binding of (i) with a level of the binding of (ii),
wherein when the level of the binding of (i) is higher or lower than the level of the binding of (ii), the subject is diagnosed as having an abnormality, disorder or disease associated with a level of axon outgrowth and/or fasciculation.
   (88) A kit for detection of a condition, disorder or disease associated with a level of axon outgrowth and/or fasciculation, comprising:

(a) at least 2 primers of at least 10 nucleotides in length, wherein each of the primers comprises:
(i) a polynucleotide having at least 70% homology to a polynucleotide set forth in SEQ ID NO. 13: and
(ii) a primer having at least 70% homology to a polynucleotide encoding an amino-acid sequence set forth in SEQ ID NO. 14 and hybridizable to the polynucleotide at different positions in the amino acid sequence under stringent conditions; and

(b) instructions describing using the primer of (a) to detect a nucleic acid sequence in a sample derived from the subject and detect a mutation in the polynucleotide set forth in SEQ ID NO. 13.

The present invention provides a marker, kit, and method for determining the level of axon outgrowth and/or fasciculation, or conditions, disorders or diseases (e.g., schizophrenia, mental retardation, etc.) associated with such a level, and a method, kit and system for identifying an agent for treatment or prophylaxis of such conditions, disorders or diseases.

### BRIEF DESCRIPTION OF THE DRAWINGS

FIG. 1 shows in situ hybridization analysis of DISC1 mRNA in rat brain. (a) Sagittal section of adult rat whole brain. Signals were observed by incubation of the sections in a visualization reagent for 6 hours at room temperature. High magnification views are shown for olfactory, hippocampal and cerebellar areas in the lower panels, respectively. OB: Olfactory bulb, CX: Cerebral cortex, Hip: Hippocampus, CB: Cerebellum, TH: Thalamus, SN: Substantia nigra. (b) Coronal sections of the hippocampus region at postnatal 7 day (left panels) and in adulthood (right panels). Signals were observed by incubation of the section in visualization reagent for 2 hours at room temperature. High magnification views are shown for hippocampal CA1 pyramidal cells (lower left and right panels). To confirm that these signals were present in neurons, the section was counterstained with thionine. DG: dentate gyrus, so: Stratum oriens, sp: Stratum pyramidale, sr: Stratum radiatum.

FIG. 2 shows detection of DISC1 protein by a specific antibody. (a) The upper portion represents the predicted protein structure of DISC1. DISC1 consists of the N-terminal globular region (black rectangle) and the helical C-terminal region (open rectangle) that contains three stretches with coiled-coil-forming potential (grey rectangles). The vertical line indicates the position of the translocation breakpoint. A polyclonal antibody was raised against the underlined sequence. The lower portion represents the splicing variant of DISC1. The triangle indicates the position of the alternatively spliced 22 amino acids. (b) Western blot by an antibody raised against DISC1 with lysates from SK-N-SH and HEK293T cells. A protein of expected size (closed arrowhead) and one of smaller size (open arrowhead) were detected. Lysates from HEK293T cells transfected with DISC1 or its splicing variant were also blotted.

FIG. 3 shows that DISC1 interacts with FEZ1. (a) Yeast two-hybrid assay to determine the binding regions of FEZ1 (upper panel) and DISC1 (lower panel). Open bars indicate the FEZ1 clone obtained by the screening and DISC1 clone used as bait. Closed bars indicate shorter fragments. Yeasts were cotransformed with the bait and one of the FEZ1 fragments, or with the FEZ1 C-terminal fragment (amino acids 247-392) and one of the DISC1 fragments. The α-galactosidase activity of yeast transformants were assayed. +++ indicates strongly positive as a positive control. ++, + and − indicate moderately positive, weakly positive and negative, respectively. (b) DISC1 coimmunoprecipitated with FEZ1. HEK293T cells were transfected with DISC1-FLAG and FEZ1-HA, individually or in combination. The FEZ1-binding region of DISC1 (amino acids 446-633, DISC1/BR) and a deleted DISC1 that lacks the binding region (DISC1/ΔBR) were also tagged with FLAG and tested in place of DISC1-FLAG. Immunoprecipitates by anti-FLAG or anti-HA antibody were blotted with the reciprocal antibody.

FIG. 4 shows intracellular localization of DISC1 and FEZ1. (a, d) SK-N-SH cells stained by anti-DISC1 or anti-FEZ1 antibody, respectively. (b, e) The cells of (a, d) also stained with phalloidin for the detection of F-actin. (c, f) Merged images of (a) with (b), (d) with (e). Arrows in (c) indicate colocalization of DISC1 and F-actin. (g, j) Cultured rat hippocampal neurons stained by anti-FEZ1 antibody. (h, k) The neurons of (g, j) also stained with phalloidin or transfected with GFP-fused DISC1, respectively. (i, l) Merged images of (g) with (h), (j) with (k). Arrows in (g-1) indicate growth cones. (m) Western blot by an antibody raised against FEZ1 with lysates from SK-N-SH cells. (n) Immunoprecipitation assay for the detection of the interaction between FEZ1 and actin. PC12 cells were transfected with FEZ1-HA or a mock, and then lysates were prepared. Immunoprecipitates by anti-HA antibody were blotted with anti-actin antibody.

FIG. 5 shows that DISC1 is involved in neurite outgrowth through its binding to FEZ1. (a) Interaction between DISC1 and FEZ1 was up-regulated during neurite outgrowth. Stably DISC1-FLAG-expressing PC12 cells were stimulated with a nerve growth factor (NGF) (50 ng/ml) for 24 hours and collected. Lysates were immunoprecipitated by anti-FLAG antibody. Immunoprecipitated complexes were subjected to SDS-PAGE and blotted with anti-FEZ1 antibody. (b-m) Stably DISC1-expressing PC12 cells (h-j and k-m showing cell line #1 and #2, respectively) exhibited enhanced extension of neurites upon the stimulation with NGF compared to mock-transfected cells (b-d and e-g showing line #1 and #2, respectively). Cells were either unstimulated (b, e, h, k) or stimulated with NGF (50 ng/ml) for 24 hours (c, f, 1,1), and 48 hours (d, g, J, m) and their neurites were subsequently microscopically observed.

FIG. 6 shows that neurite outgrowth is inhibited by overexpression of the FEZ1-binding region of DISC1. Stably DISC1-expressing PC12 cells (cell line #1 in FIG. 5) were transiently transfected with a mock pIRES2-EGFP vector (a, b) or a pIRES2-EGFP vector containing the FEZ1-binding region of DISC1 (amino acids 446-633) (c, d), and then stimulated with NGF. Transfected cells were observed for GFP fluorescence (a, c). Micrographs of the same fields are also shown (b, d). The upper and lower panels (a to d) show photographs taken in different fields.

FIG. 7 shows DISC1-KIAA interaction under infection with Adeno-KIAA-GFP and stimulation with NGF and PACAP, where mock indicates infection with a pseudogene (MOCK), and DISC1-HA#4 indicates a fourth sample infected with DISC1-HA. N indicates NGF, and P indicates PACAP.

FIG. 8 shows the expression level of DISC1-HA under stimulation with NGF and PACAP. PC12D indicates PC12 cell. DISC1-HA#4 indicates a fourth sample infected with DISC1-HA. n indicates NGF, p indicates PACAP, and np indicates stimulation with both NGF and PACAP.

FIG. 9 shows the expression level of KIAA0844GFP under infection with Adeno-KIAA-GFP and stimulation with NGF and PACAP. 12 h and 48h indicate 12 hours and 48 hours after stimulation, respectively. n indicates NGF, p indicates PACAP, and np indicates stimulation with both NGF and PACAP. Mock#4 and DISC#4 indicate fourth samples infected with a pseudogene and DISC1-HA, respectively.

FIG. 10 shows the result of Northern blotting analysis for KIAA0844 under infection with Adeno-KIAA-GFP. n indicates NGF, p indicates PACAP, and np indicates stimulation with both NGF and PACAP. Mock#4, DISC#4, and DISC#13 indicate fourth samples infected with a pseudogene and DISC1-HA, respectively.

### DESCRIPTION OF SEQUENCE LISTING

SEQ ID NO. 1 sets forth a nucleic acid sequence of human DISC1.

SEQ ID NO. 2 sets forth an amino acid sequence of human DISC1

SEQ ID NO. 3 sets forth a nucleic acid sequence of human FEZ1.

SEQ ID NO. 4 sets forth an amino acid sequence of human FEZ1.

SEQ ID NO. 5 sets forth a partial nucleic acid sequence of rat DISC1.

SEQ ID NO. 6 sets forth a partial amino acid sequence of rat DISC1.

SEQ ID NO. 7 sets forth a partial nucleic acid sequence of rat FEZ1.

SEQ ID NO. 8 sets forth a partial amino acid sequence of rat FEZ1.

SEQ ID NO. 9 sets forth a partial nucleic acid sequence of mouse DISC1.

SEQ ID NO. 10 sets forth a partial amino acid sequence of mouse DISC1.

SEQ ID NO. 11 sets forth the nucleic acid sequence of a primer 1 used in Example 1.

SEQ ID NO. 12 sets forth the nucleic acid sequence of a primer 2 used in Example 1.

SEQ ID NO. 13 sets forth a nucleic acid sequence of KIAA0844.

SEQ ID NO. 14 sets forth an amino acid sequence of KIAA0844.

SEQ ID NO. 15 sets forth a nucleic acid sequence of DISC1-HA used in Example 7.

SEQ ID NO. 16 sets forth an amino acid sequence of DISC1-HA used in Example 7.

SEQ ID NO. 17 sets forth a nucleic acid sequence of KIAA-GFP used in Example 7.

SEQ ID NO. 18 sets forth a nucleic acid sequence of KIAA-GFP used in Example 7.

SEQ ID NO. 19 sets forth a partial sequence of KIAA0844 used as a probe in Example 10.

### BEST MODE FOR CARRYING OUT THE INVENTION

Hereinafter, the present invention will be described. It should be understood throughout the present specification that singular forms include plural referents unless the context clearly specifies otherwise. It should be also understood that the terms as used herein have definitions typically used in the art unless otherwise mentioned.

(DISC1)

As used herein, the term “DISC1 (Disrupted-in-Schizophrenia 1)” refers to a gene identified as a gene disrupted by a (1; 11)(q42.1; q14.3) translocation associated with schizophrenia, and a homologue thereof and a corresponding gene. Representatively, DISC1 comprises:

(a) a polynucleotide having a base sequence set forth in SEQ ID NO. 1 or a fragment thereof;

(b) a polynucleotide encoding a polypeptide consisting of an amino acid sequence set forth in SEQ ID NO. 2 or a fragment thereof;

(c) a polynucleotide encoding a variant polypeptide having an amino acid sequence set forth in SEQ ID NO. 2 or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 1;

(e) a polynucleotide encoding a species homolog of a polypeptide consisting of an amino acid sequence set forth in SEQ ID NO. 2;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity.

Preferably, DISC1 is a nucleic acid molecule consisting of the sequence set forth in SEQ ID NO. 1 or a polypeptide consisting of the amino acid sequence set forth in SEQ ID NO. 2.

DISC1 was identified as a novel gene disrupted by a (1:11)(q42.1;q14.3) translocation associated with schizophrenia in a Scottish family. DISC1 product has no significant homology to other known proteins.

The present inventors demonstrated the existence of the DISC1 protein and identified the fasciculation and elongation protein zeta-1 (FEZ1) as an interacting partner of DISC1 by a yeast two-hybrid study. FEZ1 and its nematode homologue have been reported to represent a new protein family involved in axonal outgrowth and fasciculation. In the present invention, it was revealed that DISC1 and FEZ1 colocalized in growth cones of cultured hippocampal neurons. It was found that interactions of these proteins were associated with F-actin. In the course of neuronal differentiation of PC12 cells, up-regulation of DISC1/FEZ1 interaction was observed as along with enhanced extension of neurites by overexpression of DISC1. The present invention shows that DISC1 participates in neurites outgrowth through its interaction with FEZ1. Therefore, the present invention provided reliable evidence that schizophrenia is a neurodevelopmental disorder. As there is a high level of DISC1 expression in developing rat brains, dysfunction of DISC1 may confer susceptibility to psychiatric illnesses through abnormal development of the nervous system. We first demonstrated that the expression level of DISC1 was enhanced in developing rat brains. Further, the presence of DISC1 protein was demonstrated using specific antibodies. Furthermore, we first demonstrated that FEZ1 was as an interacting partner of DISC1. The interaction of DISC1 and FEZ1 was associated with actin cytoskeleton and up-regulated during neurite outgrowth.

(FEZ1)

As used herein, the term “FEZ1” (fasciculation and elongation protein zeta-1) refers to a mammalian animal homologue of Caenorhabditis elegans UNC-76 protein which is involved in axon outgrowth and fasciculation, and the corresponding gene. FEZ1 and a homologue thereof have been reported to represent a novel protein family involved in axon outgrowth and fasciculation. Representatively, FEZ1 comprises:

(a) a polynucleotide having a base sequence set forth in SEQ ID NO. 3 or a fragment thereof;

(b) a polynucleotide encoding a polypeptide consisting of an amino acid sequence set forth in SEQ ID NO. 4 or a fragment thereof;

(c) a polynucleotide encoding a variant polypeptide having an amino acid sequence set forth in SEQ ID NO. 4 or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 3;

(e) a polynucleotide encoding a species homolog of a polypeptide consisting of an amino acid sequence set forth in SEQ ID NO. 4;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity.

Preferably, FEZ1 is a nucleic acid molecule consisting of the sequence set forth in SEQ ID NO. 3 or a polypeptide consisting of the amino acid sequence set forth in SEQ ID NO. 4.

(KIAA0844)

As Used Herein, the Term “KIAA0844” Refers to a Human gene which was first sequenced in Nagase T. et al., DNA Res., 1998, 5(6):355-64. Representatively, KIAA0844 comprises:

(a) a polynucleotide having a base sequence set forth in SEQ ID NO. 13 or a fragment thereof;

(b) a polynucleotide encoding a polypeptide consisting of an amino acid sequence set forth in SEQ ID NO. 14 or a fragment thereof;

(c) a polynucleotide encoding a variant polypeptide having an amino acid sequence set forth in SEQ ID NO. 14 or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 13;

(e) a polynucleotide encoding a species homolog of a polypeptide consisting of an amino acid sequence set forth in SEQ ID NO. 14;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity.

Alternatively, KIAA0844 comprises a sequence contained in data indicated by ACCESSION NO. 094930.

Preferably, KIAA0844 is a nucleic acid molecule consisting of the sequence set forth in SEQ ID NO. 13 or a polypeptide consisting of the amino acid sequence set forth in SEQ ID NO. 14.

The function of KIAA0844 has not been clarified before the present invention. The present invention is the first to elucidate the relationship between KIAA0844 and DISC1 and/FEZ1. Thus, in the present invention, it was revealed that KIAA0844 is associated with axon outgrowth and/or fasciculation.

(Definition of Terms)

Hereinafter, terms specifically used herein are defined.

The terms “protein”, “polypeptide”, “oligopeptide” and “peptide” as used herein have the same meaning and refer to an amino acid polymer having any length. This polymer may be a straight, branched or cyclic chain. An amino acid may be a naturally-occurring or non-naturally-occurring amino acid, or a variant amino acid. The term may include those assembled into a composite of a plurality of polypeptide chains. The term also includes a naturally-occurring or artificially modified amino acid polymer. Such modification includes, for example, disulfide bond formation, glycosylation, lipidation, acetylation, phosphorylation, or any other manipulation or modification. (e.g., conjugation with a labeling moiety). This definition encompasses a polypeptide containing at least one amino acid analog (e.g., non-naturally-occurring amino acid, etc.), a peptide-like compound (e.g., peptoid), and other variants known in the art, for example. Gene products of DISC1 and FEZ1 are typically in the form of a polypeptide.

The terms “polynucleotide”, “oligonucleotide”, and “nucleic acid” as used herein have the same meaning and refer to a nucleotide polymer having any length. This term also includes an “oligonucleotide derivative” or a “polynucleotide derivative”. An “oligonucleotide derivative” or a “polynucleotide derivative” includes a nucleotide derivative, or refers to an oligonucleotide or a polynucleotide having different linkages between nucleotides from typical linkages, which are interchangeably used. Examples of such an oligonucleotide specifically include 2′-O-methyl-ribonucleotide, an oligonucleotide derivative in which a phosphodiester bond in an oligonucleotide is converted to a phosphorothioate bond, an oligonucleotide derivative in which a phosphodiester bond in an oligonucleotide is converted to a N3′-P5′ phosphoroamidate bond, an oligonucleotide derivative in which a ribose and a phosphodiester bond in an oligonucleotide are converted to a peptide-nucleic acid bond, an oligonucleotide derivative in which uracil in an oligonucleotide is substituted with C-5 propynyl uracil, an oligonucleotide derivative in which uracil in an oligonucleotide is substituted with C-5 thiazole uracil, an oligonucleotide derivative in which cytosine in an oligonucleotide is substituted with C-5 propynyl cytosine, an oligonucleotide derivative in which cytosine in an oligonucleotide is substituted with phenoxazine-modified cytosine, an oligonucleotide derivative in which ribose in DNA is substituted with 2′-O-propyl ribose, and an oligonucleotide derivative in which ribose in an oligonucleotide is substituted with 2′-methoxyethoxy ribose. Unless otherwise indicated, a particular nucleic acid sequence also implicitly encompasses conservatively-modified variants thereof (e.g. degenerate codon substitutions) and complementary sequences as well as the sequence explicitly indicated. Specifically, degenerate codon substitutions may be produced by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). Genes, such as DISC1, FEZ1, and the like, are typically in the form of a polynucleotide.

As used herein, the term “nucleic acid molecule” is used interchangeably with “nucleic acid”, “oligonucleotide”, and “polynucleotide”, including cDNA, mRNA, genomic DNA, and the like. As used herein, nucleic acid and nucleic acid molecule may be included by the concept of the term “gene”. A nucleic acid molecule encoding the sequence of a given gene includes “splice mutant (variant)”. Similarly, a particular protein encoded by a nucleic acid encompasses any protein encoded by a splice variant of that nucleic acid. “Splice mutants”, as the name suggests, are products of alternative splicing of a gene. After transcription, an initial nucleic acid transcript may be spliced such that different (alternative) nucleic acid splice products encode different polypeptides. Mechanisms for the production of splice variants vary, but include alternative splicing of exons. Alternative polypeptides derived from the same nucleic acid by read-through transcription are also encompassed by this definition. Any products of a splicing reaction, including recombinant forms of the splice products, are included in this definition. Therefore, herein, a DISC1 gene may also include a spliced mutant of DISC1, for example.

As used herein, the term “gene” refers to an element defining a genetic trait. A gene is typically arranged in a given sequence on a chromosome. A gene which defines the primary structure of a protein is called a structural gene. A gene which regulates the expression of a structural gene is called a regulatory gene (e.g., promoter). Genes herein include structural genes and regulatory genes unless otherwise specified. Therefore, a DISC1 gene typically includes a structure gene of DISC1, a promoter of DISC1, and a regulatory factor associated therewith. As used herein, “gene” may refer to “polynucleotide”, “oligonucleotide”, “nucleic acid”, and “nucleic acid molecule” and/or “protein”, “polypeptide”, “oligopeptide” and “peptide”. As used herein, “gene product” includes “polynucleotide”, “oligonucleotide”, “nucleic acid” and “nucleic acid molecule” and/or “protein”, “polypeptide”, “oligopeptide” and “peptide”, which are expressed by a gene. Those skilled in the art will understand what a gene product is, according to the context.

As used herein, the term “homology” in relation to a gene (e.g., a nucleic acid sequence, an amino acid sequence, etc.) refers to the proportion of identity between two or more gene sequences. Therefore, the greater the homology between two given genes, the greater the identity or similarity between their sequences. Whether or not two genes have homology is determined by comparing their sequences directly or by a hybridization method under stringent conditions. When two gene sequences are directly compared with each other, these genes have homology if the DNA sequences of the genes have representatively at least 50% identity, preferably at least 70. % identity, more preferably at least 80%, 90%, 95%, 96%, 97%, 98%, or 99% identity with each other. As used herein, the term “similarity” in relation to a gene (e.g., a nucleic acid sequence, an amino acid sequence, or the like) refers to the proportion of identity between two or more sequences when conservative substitution is regarded as positive (identical) in the above-described homology. Therefore, homology and similarity differ from each other in the presence of conservative substitutions. If no conservative substitutions are present, homology and similarity have the same value.

The similarity, identity and homology of amino acid sequences and base sequences are herein compared using BLAST (sequence analyzing tool) with the default parameters.

As used herein, the term “amino acid” may refer to a naturally-occurring or non-naturally-occurring amino acid as long as the object of the present invention is satisfied.

As used herein, the term “amino acid derivative” or “amino acid analog” refers to an amino acid which is different from a naturally-occurring amino acid and has a function similar to that of the original amino acid. Such amino acid derivatives and amino acid analogs are well known in the art.

The term “naturally-occurring amino acid” refers to an L-isomer of a naturally-occurring amino acid. The naturally-occurring amino acids are glycine, alanine, valine, leucine, isoleucine, serine, methionine, threonine, phenylalanine, tyrosine, tryptophan, cysteine, proline, histidine, aspartic acid, asparagine, glutamic acid, glutamine, γ-carboxyglutamic acid, arginine, ornithine, and lysine. Unless otherwise indicated, all amino acids as used herein are L-isomers. An embodiment using a D-isomer of an amino acid falls within the scope of the present invention.

The term “non-naturally-occurring amino acid” refers to an amino acid which is ordinarily not found in nature. Examples of non-naturally-occurring amino acids include D-form of an amino acid as described above, norleucine, para-nitrophenylalanine, homophenylalanine, para-fluorophenylalanine, 3-amino-2-benzyl propionic acid, D- or L-homoarginine, and D-phenylalanine.

As used herein, the term “amino acid analog” refers to a molecule having a physical property and/or function similar to that of amino acids, but is not an amino acid. Examples of amino acid analogs include, for example, ethionine, canavanine, 2-methylglutamine, and the like. An amino acid mimic refers to a compound which has a structure different from that of the general chemical structure of amino acids but which functions in a manner similar to that of naturally-occurring amino acids.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the term “corresponding” amino acid or nucleic acid refers to an amino acid or nucleotide in a given polypeptide or polynucleotide molecule, which has, or is anticipated to have, a function similar to that of a predetermined amino acid or nucleotide in a polypeptide or polynucleotide as a reference for comparison. Particularly, in the case of enzyme molecules, the term refers to an amino acid which is present at a similar position in an active site and similarly contributes to catalytic activity. For example, in the case of an antisense molecule, a corresponding antisense molecule may be a similar portion in an ortholog corresponding to a particular portion of the antisense molecule. For DISC1 or FEZ of the present invention, a corresponding amino acid or nucleic acid may be a site which interacts with FEZ or DISC1 or a site encoding it, respectively, for example. In another embodiment, for DISC1 or FEZ of the present invention, a corresponding amino acid may be an amino acid which plays a role in complexation. Such a “corresponding” amino acid or nucleic acid may be a region or domain extending over a certain range. Therefore, such a region or domain is herein referred to as a “corresponding” region or domain.

As used herein, the term “corresponding” gene (e.g., a polypeptide or polynucleotide molecule) refers to a gene in a given species, which has, or is anticipated to have, a function similar to that of a predetermined gene in a species as a reference for comparison. When there are a plurality of genes having such a function, the term refers to a gene having the same evolutionary origin. Therefore, a gene corresponding to a given gene may be an ortholog of the given gene. Thus, a gene corresponding to a mouse DISC1 gene can be found in other animals. Such a corresponding gene can be identified by techniques well known in the art. For example, a corresponding gene in a given animal can be found by searching a sequence database of the animal (e.g., human, rat) using the sequence of a reference gene (e.g., a mouse DISC1 gene, etc.) as a query sequence.

As used herein, the term “nucleotide” may be either naturally-occurring or non-naturally-occurring. The term “nucleotide derivative” or “nucleotide analog” refers to a nucleotide which is different from naturally-occurring nucleotides and has a function similar to that of the original nucleotide. Such nucleotide derivatives and nucleotide analogs are well known in the art. Examples of such nucleotide derivatives and nucleotide analogs include, but are not limited to, phosphorothioate, phosphoramidate, methylphosphonate, chiral-methylphosphonate, 2-O-methyl ribonucleotide, and peptide-nucleic acid (PNA).

As used herein, the term “fragment” with respect to a polypeptide or polynucleotide refers to a polypeptide or polynucleotide having a sequence length ranging from 1 to n−1 with respect to the full length of the reference polypeptide or polynucleotide (of length n). The length of the fragment can be appropriately changed depending on the purpose. For example, in the case of polypeptides, the lower limit of the length of the fragment includes 3, 4, 5, 6, 7, 8, 9, 10, 15, 20, 25, 30, 40, 50 or more nucleotides. Lengths represented by integers which are not herein specified (e.g., 11 and the like) may be appropriate as a lower limit. For example, in the case of polynucleotides, the lower limit of the length of the fragment includes 5, 6, 7, 8, 9, 10, 15, 20, 25, 30, 40, 50, 75, 100 or more nucleotides. Lengths represented by integers which are not herein specified (e.g., 11 and the like) may be appropriate as a lower limit. As used herein, the length of polypeptides or polynucleotides can be represented by the number of amino acids or nucleic acids, respectively. However, the above-described numbers are not absolute. The above-described numbers, as the upper or lower limit, are intended to include some greater or smaller numbers (e.g., 10%), as long as the same function is maintained. For this purpose, “about” may be herein put ahead of the numbers. However, it should be understood that the interpretation of numbers is not affected by the presence or absence of “about” in the present specification.

As used herein, the term “agent specifically interacting with” a biological agent, such as a polynucleotide, a polypeptide or the like, refers to an agent which has an affinity for the biological agent, such as a polynucleotide, a polypeptide or the like, which is representatively higher than or equal to the affinity for other non-related biological agents, such as polynucleotides, polypeptides or the like (particularly, those with identity of less than 30%), and preferably significantly (e.g., statistically significantly) higher. Such an affinity can be measured with, for example, a hybridization assay, a binding assay, or the like. As used herein, the “agent” may be any substance or other agent (e.g., energy) as long as the intended purpose can be achieved. Examples of such a substance include, but are not limited to, proteins, polypeptides, oligopeptides, peptides, polynucleotides, oligonucleotides, nucleotides, nucleic acids (e.g., DNA such as cDNA, genomic DNA, or the like, and RNA such as mRNA), polysaccharides, oligosaccharides, lipids, low molecular weight organic molecules (e.g., hormones, ligands, information transfer substances, molecules synthesized by combinatorial chemistry, low molecular weight molecules (e.g., pharmaceutically acceptable low molecular weight ligands and the like), and the like), and combinations of these molecules. Examples of an agent specific to a polynucleotide include, but are not limited to, a polynucleotide having complementarity to the sequence of the polynucleotide with a predetermined sequence homology (e.g., 70% or more sequence identity), a polypeptide such as a transcriptional agent binding to a promoter region, and the like. Examples of an agent specific to a polypeptide include, but are not limited to, an antibody specifically directed to the polypeptide or derivatives or analogs thereof (e.g., single chain antibody), a specific ligand or receptor when the polypeptide is a receptor or ligand, a substrate when the polypeptide is an enzyme, and the like.

As used herein, the term “low molecular weight organic molecules” refers to an organic molecule having a relatively small molecular weight. Usually, the low molecular weight organic molecule refers to a molecular weight of about 1,000 or less, or may refer to a molecular weight of more than 1,000. Low molecular weight organic molecules can be ordinarily synthesized by methods known in the art or combinations thereof. These low molecular weight organic molecules may be produced by organisms. Examples of the low molecular weight organic molecule include, but are not limited to, hormones, ligands, information transfer substances, synthesized by combinatorial chemistry, pharmaceutically acceptable low molecular weight molecules (e.g., low molecular weight ligands and the like), and the like.

As used herein, the term “antibody” encompasses polyclonal antibodies, monoclonal antibodies, human antibodies, humanized antibodies, polyfunctional antibodies, chimeric antibodies, and anti-idiotype antibodies, and fragments thereof (e.g., F(ab′)2 and Fab fragments), and other recombinant conjugates. These antibodies may be fused with an enzyme (e.g., alkaline phosphatase, horseradish peroxidase, α-galactosidase, and the like) via a covalent bond or by recombination.

As used herein, the term “monoclonal antibody” refers to an antibody composition having a group of homologous antibodies. This term is not limited by the production manner thereof. This term encompasses all immunoglobulin molecules and Fab molecules, F(ab′) 2 fragments, Fv fragments, and other molecules having an immunological binding property of the original monoclonal antibody molecule. Methods for producing polyclonal antibodies and monoclonal antibodies are well known in the art, and will be more sufficiently described below.

Monoclonal antibodies are prepared by using a standard technique well known in the art (e.g., Kohler and Milstein, Nature, 1975, 256:495) or a modification thereof (e.g., Buck et al., In Vitro, 18, 1982:377). Representatively, a mouse or rat is immunized with a protein bound to a protein carrier, and boosted. Subsequently, the spleen (and optionally several large lymph nodes) is removed and dissociated into single cells. If desired, the spleen cells may be screened (after removal of nonspecifically adherent cells) by applying a cell suspension to a plate or well coated with a protein antigen. B-cells that express membrane-bound immunoglobulin specific for the antigen bind to the plate, and are not rinsed away with the rest of the suspension. Resulting B-cells, or all dissociated spleen cells, are then induced to fuse with myeloma cells to form hybridomas. The hybridomas are used to produce monoclonal antibodies.

As used herein, the term “antigen” refers to any substrate to which an antibody molecule may specifically bind. As used herein, the term “immunogen” refers to an antigen initiating activation of the antigen-specific immune response of a lymphocyte.

As used herein, the term “single chain antibody” refers to a single chain polypeptide formed by linking a heavy chain fragment and the light chain fragment of the Fv region via peptide crosslinker.

As used herein, the term “composite molecule” refers to a molecule in which a plurality of molecules, such as polypeptides, polynucleotides, lipids, sugars, small molecules, or the like, are linked together. Examples of a composite molecule include, but are not limited to, glycolipids, glycopeptides, and the like. Such composite molecules can be herein used as a DICS1 gene or a product thereof, or an agent of the present invention, as long as they have a similar function to that of the DICS1 gene or the product thereof, or the agent of the present invention.

As used herein, the term “isolated” biological agent (e.g., nucleic acid, protein, or the like) refers to a biological agent that is substantially separated or purified from other biological agents in cells of a naturally-occurring organism (e.g., in the case of nucleic acids, agents other than nucleic acids and a nucleic acid having nucleic acid sequences other than an intended nucleic acid; and in the case of proteins, agents other than proteins and proteins having an amino acid sequence other than an intended protein). The “isolated” nucleic acids and proteins include nucleic acids and proteins purified by a standard purification method. The isolated nucleic acids and proteins also include chemically synthesized nucleic acids and proteins.

As used herein, the term “purified” biological agent (e.g., nucleic acids, proteins, and the like) refers to one from which at least a part of naturally accompanying agent is removed. Therefore, ordinarily, the purity of a purified biological agent is higher than that of the biological agent in a normal state (i.e., concentrated).

As used herein, the terms “purified” and “isolated” mean that the same type of biological agent is present preferably at least 75% by weight, more preferably at least 85% by weight, even more preferably at least 95% by weight, and most preferably at least 98% by weight.

As used herein, the term “expression” of a gene, a polynucleotide, a polypeptide, or the like, indicates that the gene or the like is affected by a predetermined action in vivo to be changed into another form. Preferably, the term “expression” indicates that genes, polynucleotides, or the like are transcribed and translated into polypeptides. In one embodiment of the present invention, genes may be transcribed into mRNA. More preferably, these polypeptides may have post-translational processing modifications.

Therefore, as used herein, the term “reduction” of “expression” of a gene, a polynucleotide, a polypeptide, or the like indicates that the level of expression is significantly reduced in the presence of or under the action of the agent of the present invention as compared to when the action of the agent is absent. Preferably, the reduction of expression includes a reduction in the amount of expression of a polypeptide. As used herein, the term “increase” of “expression” of a gene, a polynucleotide, a polypeptide, or the like indicates that the level of expression is significantly increased in the presence of the action of the agent of the present invention as compared to when the action of the agent is absent. Preferably, the increase of expression includes an increase in the amount of expression of a polypeptide. As used herein, the term “induction” of “expression” of a gene indicates that the amount of expression of the gene is increased by applying a given agent to a given cell. Therefore, the induction of expression includes allowing a gene to be expressed when expression of the gene is not otherwise observed, and increasing the amount of expression of the gene when expression of the gene is observed.

As used herein, the term “specifically expressed” in relation to a gene indicates that the gene is expressed in a specific site or for a specific period of time, at a level different from (preferably higher than) that in other sites or for other periods of time. The term “specifically expressed” indicates that a gene may be expressed only in a given site (specific site) or may be expressed in other sites. Preferably, the term “specifically expressed” indicates that a gene is expressed only in a given site.

As used herein, the term “biological activity” refers to activity possessed by an agent (e.g., a polynucleotide, a protein, etc.) within an organism, including activities exhibiting various functions (e.g., transcription promoting activity, etc.). For example, when two agents interact with each other (e.g., DISC1 is coupled with FEZ1), the biological activity thereof includes the binding of FEZ1 with DISC1 and a biological change (e.g., axon outgrowth and/or fasciculation, etc.) caused thereby. In another example, when a certain factor is an enzyme, the biological activity thereof includes its enzyme activity. In still another example, when a certain factor is a ligand, the biological activity thereof includes the binding of the ligand to a receptor corresponding thereto. The above-described biological activity can be measured by techniques well-known in the art.

As used herein, the term “antisense (activity)” refers to activity which permits specific suppression or reduction of expression of a target gene. The antisense activity is ordinarily achieved by a nucleic acid sequence having a length of at least 8 contiguous nucleotides, which is complementary to the nucleic acid sequence of a target gene (e.g., DISC1, FEZ1, KIAA0844, etc.). A molecule having such antisense activity is called an antisense molecule. Such a nucleic acid sequence preferably has a length of at least 9 contiguous nucleotides, more preferably a length of at least 10 contiguous nucleotides, and even more preferably a length of at least 11 contiguous nucleotides, a length of at least 12 contiguous nucleotides, a length of at least 13 contiguous nucleotides, a length of at least 14 contiguous nucleotides, a length of at least 15 contiguous nucleotides, a length of at least 20 contiguous nucleotides, a length of at least 30 contiguous nucleotides, a length of at least 40 contiguous nucleotides, and a length of at least 50 contiguous nucleotides. These nucleic acid sequences include nucleic acid sequences having at least 70% homology thereto, more preferably at least 80%, even more preferably at least 90%, and still even more preferably at least 95%. The antisense activity is preferably complementary to a 5′ terminal sequence of the nucleic acid sequence of a target gene. Such an antisense nucleic acid sequence includes the above-described sequences having one or several, or at least one, nucleotide substitutions, additions, and/or deletions.

General antisense techniques are described in textbooks (e.g., Murray, J. A. H. eds., Antisense RNA and DNA, Wiley-Liss Inc, 1992). Later research has revealed a phenomenon called RNA interference (RNAi), leading to the development of antisense techniques. RNAi is a phenomenon in which when double-stranded RNA (about 20 bases in length) having a sequence homologous to a target gene is introduced into a cell, mRNA of the target gene homologous to the RNA sequence is specifically decomposed to reduce the expression level thereof. The phenomenon which was originally found in nematodes has been revealed to be a universal phenomenon throughout organisms including plants. The molecular mechanism of the antisense technique suppressing the expression of target genes has been elucidated to have a process similar to that of RNAi. Conventionally, a certain DNA sequence complementary to the nucleotide sequence of a target gene is linked to an appropriate promoter to construct an expression vector which expresses artificial mRNA under the control of a promoter, and the vector is then introduced into cells. According to recent findings, an expression vector which is designed to construct double-stranded RNA in cells is used. The basic structure of the vector is such that a DNA sequence complementary to a certain target gene is linked downstream of a promoter and the same sequence is linked in the reverse direction. A single-stranded mRNA transcribed from the above-described constructed gene is paired with the reverse-directed, complementary nucleotide sequence portion into double-stranded RNA having a hair-pin, secondary structure. This structure elicits decomposition of mRNA of a target gene in accordance with the mechanism of RNAi. RNAi is reviewed in, for example, Morita and Yoshida, Tanpakushitsu•Kakusan•Koso [Protein/Nucleic acid/Enzyme], 47, 1939-1945, 2002). These documents are herein incorporated by reference in their entirety.

As used herein, the term “RNAi” is an abbreviation of RNA interference and refers to a phenomenon where an agent for causing RNAi, such as double-stranded RNA (also called dsRNA), is introduced into cells and mRNA homologous thereto is specifically degraded, so that synthesis of gene products is suppressed, and a technique using the phenomenon. As used herein, RNAi may have the same meaning as that of an agent which causes RNAi.

As used herein, the term “an agent causing RNAi” refers to any agent causing RNAi. As used herein, “an agent causing RNAi for a gene” indicates that the agent causes RNAi relating to the gene and the effect of RNAi is achieved (e.g., suppression of expression of the gene, and the like). Examples of such an agent causing RNAi include, but are not limited to, a sequence having at least about 70% homology to the nucleic acid sequence of a target gene or a sequence hybridizable under stringent conditions, RNA containing a double-stranded portion having a length of at least 10 nucleotides or variants thereof. Herein, this agent may be preferably DNA containing a 3′ protruding end, and more preferably the 3′ protruding end has a length of 2 or more nucleotides (e.g., 2-4 nucleotides in length).

Alternatively, RNAi used in the present invention is, for example, but is not limited to, a pair of short reverse-directed, complementary sequences (e.g., 15 bp or more, for example, 23 bp, etc.).

As used herein, “polynucleotides hybridizing under stringent conditions” refers to conditions commonly used and well known in the art. Such a polynucleotide can be obtained by conducting colony hybridization, plaque hybridization, Southern blot hybridization, or the like using a polynucleotide selected from the polynucleotides of the present invention. Specifically, a filter, on which DNA derived from a colony or plaque is immobilized, is used to conduct hybridization at 65° C. in the presence of 0.7 to 1.0 M NaCl. Thereafter, a 0.1 to 2-fold concentration SSC (saline-sodium citrate) solution (1-fold concentration SSC solution is composed of 150 mM sodium chloride and 15 mM sodium citrate) is used to wash the filter at 65° C. Polynucleotides identified by this method are referred to as “polynucleotides hybridizing under stringent conditions”. Hybridization can be conducted in accordance with a method described in, for example, Molecular Cloning 2nd ed., Current Protocols in Molecular Biology, Supplement 1-38, DNA Cloning 1: Core Techniques, A Practical Approach, Second Edition, Oxford University Press (1995), and the like. Here, sequences hybridizing under stringent conditions exclude, preferably, sequences containing only A or T. “Hybridizable polynucleotide” refers to a polynucleotide which can hybridize other polynucleotides under the above-described hybridization conditions. Specifically, the hybridizable polynucleotide includes at least a polynucleotide having a homology of at least 60% to the base sequence of DNA encoding a polypeptide having an amino acid sequence specifically disclosed herein, preferably a polynucleotide having a homology of at least 80%, and more preferably a polynucleotide having a homology of at least 95%.

As used herein, the term “probe” refers to a substance for use in searching, which is used in a biological experiment, such as in vitro and/or in vivo screening or the like, including, but not being limited to, for example, a nucleic acid molecule having a specific base sequence or a peptide containing a specific amino acid sequence.

Examples of a nucleic acid molecule as a common probe include one having a nucleic acid sequence having a length of at least 8 contiguous nucleotides, which is homologous or complementary to the nucleic acid sequence of a gene of interest. Such a nucleic acid sequence may be preferably a nucleic acid sequence having a length of at least 9 contiguous nucleotides, more preferably a length of at least 10 contiguous nucleotides, and even more preferably a length of at least 11 contiguous nucleotides, a length of at least 12 contiguous nucleotides, a length of at least 13 contiguous nucleotides, a length of at least 14 contiguous nucleotides, a length of at least 15 contiguous nucleotides, a length of at least 20 contiguous nucleotides, a length of at least 25 contiguous nucleotides, a length of at least 30 contiguous nucleotides, a length of at least 40 contiguous nucleotides, or a length of at least 50 contiguous nucleotides. A nucleic acid sequence used as a probe includes a nucleic acid sequence having at least 70% homology to the above-described sequence, more preferably at least 80%, and even more preferably at least 90% or at least 95%.

As used herein, the term “search” indicates that a given nucleic acid sequence is utilized to find other nucleic acid base sequences having a specific function and/or property either electronically or biologically, or using other methods. Examples of an electronic search include, but are not limited to, BLAST (Altschul et al., J. Mol. Biol. 215:403-410 (1990)), FASTA (Pearson & Lipman, Proc. Natl. Acad. Sci., USA 85:2444-2448 (1988)), Smith and Waterman method (Smith and Waterman, J. Mol. Biol. 147:195-197 (1981)), and Needleman and Wunsch method (Needleman and Wunsch, J. Mol. Biol. 48:443-453 (1970)), and the like. Examples of a biological search include, but are not limited to, stringent hybridization, a macroarray in which genomic DNA is attached to a nylon membrane or the like or a microarray (microassay) in which genomic DNA is attached to a glass plate, PCR and In situ hybridization conditions, and the like.

As used herein, the term “primer” refers to a substance required for initiation of a reaction of a macromolecule compound to be synthesized, in a macromolecule synthesis enzymatic reaction. In a reaction for synthesizing a nucleic acid molecule, a nucleic acid molecule (e.g., DNA, RNA, or the like) which is complementary to part of a macromolecule compound to be synthesized may be used.

A nucleic acid molecule which is ordinarily used herein as a primer includes one that has a nucleic acid sequence having a length of at least 8 contiguous nucleotides, which is complementary to the nucleic acid sequence of a gene of interest. Such a nucleic acid sequence preferably has a length of at least 9 contiguous nucleotides, more preferably a length of at least 10 contiguous nucleotides, even more preferably a length of at least 11 contiguous nucleotides, a length of at least 12 contiguous nucleotides, a length of at least 13 contiguous nucleotides, a length of at least 14 contiguous nucleotides, a length of at least 15 contiguous nucleotides, a length of at least 16 contiguous nucleotides, a length of at least 17 contiguous nucleotides, a length of at least 18 contiguous nucleotides, a length of at least 19 contiguous nucleotides, a length of at least 20 contiguous nucleotides, a length of at least 25 contiguous nucleotides, a length of at least 30 contiguous nucleotides, a length of at least 40 contiguous nucleotides, and a length of at least 50 contiguous nucleotides. A nucleic acid sequence used as a primer includes a nucleic acid sequence having at least 70% homology to the above-described sequence, more preferably at least 80%, even more preferably at least 90%, and most preferably at least 95%. An appropriate sequence as a primer may vary depending on the property of the sequence to be synthesized (amplified). Those skilled in the art can design an appropriate primer depending on the sequence of interest. Such primer design is well known in the art and may be performed manually or using a computer program (e.g., LASERGENE, Primer Select, DNAStar).

As used herein, the term “epitope” refers to an antigenic determinant. Therefore, the term “epitope” includes a set of amino acid residues which is involved in recognition by a particular immunoglobulin, or in the context of T cells, those residues necessary for recognition by T cell receptor proteins and/or Major Histocompatibility Complex (MHC) receptors. This term is also used interchangeably with “antigenic determinant” or “antigenic determinant site”. In the field of immunology, in vivo or In vitro, an epitope is a feature of a molecule (e.g., primary, secondary and tertiary peptide structure, and charge) that forms a site recognized by an immunoglobulin, T cell receptor or HLA molecule. An epitope including a peptide comprises 3 or more amino acids in a spatial conformation which is unique to the epitope. Generally, an epitope consists of at least 5 such amino acids, and more ordinarily, consists of at least 6, 7, 8, 9 or 10 such amino acids. The greater the length of an epitope, the more the similarity of the epitope to the original peptide, i.e., longer epitopes are generally preferable. This is not necessarily the case when the conformation is taken into account. Methods of determining the spatial conformation of amino acids are known in the art, and include, for example, X-ray crystallography and 2-dimensional nuclear magnetic resonance spectroscopy. Furthermore, the identification of epitopes in a given protein is readily accomplished using techniques well known in the art. See, also, Geysen et al., Proc. Natl. Acad. Sci. USA (1984) 81: 3998 (general method of rapidly synthesizing peptides to determine the location of immunogenic epitopes in a given antigen); U.S. Pat. No. 4,708,871 (procedures for identifying and chemically synthesizing epitopes of antigens); and Geysen et al., Molecular immunology (1986) 23: 769 (technique for identifying peptides with high affinity for a given antibody). Antibodies that recognize the same epitope can be identified in a simple immunoassay. Thus, methods for determining an epitope including a peptide, are well known in the art. Such an epitope can be determined using a well-known, common technique by those skilled in the art if the primary nucleic acid or amino acid sequence of the epitope is provided.

Therefore, an epitope including a peptide requires a sequence having a length of at least 3 amino acids, preferably at least 4 amino acids, more preferably at least 5 amino acids, at least 6 amino acids, at least 7 amino acids, at least 8 amino acids, at least 9 amino acids, at least 10 amino acids, at least 15 amino acids, at least 20 amino acids, and 25 amino acids. Epitopes may be linear or conformational.

(Modification of Genes)

As used herein, the present invention can be used in a modified form. In a given protein molecule, a given amino acid contained in a sequence may be substituted with another amino acid in a protein structure, such as a cationic region or a substrate molecule binding site, without a clear reduction or loss of interactive binding ability. A given biological function of a protein is defined by the interactive ability or other property of the protein. Therefore, a particular amino acid substitution may be performed in an amino acid sequence, or at the DNA code sequence level, to produce a protein which maintains the original property after the substitution. Therefore, various modifications of peptides as disclosed herein and DNA encoding such peptides may be performed without clear losses of biological usefulness.

When the above-described modifications are designed, the hydrophobicity indices of amino acids may be taken into consideration. The hydrophobic amino acid indices play an important role in providing a protein with an interactive biological function, which is generally recognized in the art (Kyte, J. and Doolittle, R. F., J. Mol. Biol. 157(1):105-132, 1982). The hydrophobic property of an amino acid contributes to the secondary structure of a protein and then regulates interactions between the protein and other molecules (e.g., enzymes, substrates, receptors, DNA, antibodies, antigens, etc.). Each amino acid is given a hydrophobicity index based on the hydrophobicity and charge properties thereof as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamic acid (−3.5); glutamine (−3.5); aspartic acid (−3.5); asparagine (−3.5); lysine (−3.9); and arginine (−4.5).

It is well known that if a given amino acid is substituted with another amino acid having a similar hydrophobicity index, the resultant protein may still have a biological function similar to that of the original protein (e.g., a protein having an equivalent enzymatic activity). For such an amino acid substitution, the hydrophobicity index is preferably within ±2, more preferably within ±1, and even more preferably within ±0.5. It is understood in the art that such an amino acid substitution based on hydrophobicity is efficient.

It is herein understood that a hydrophilicity index is also useful for modification of proteins. As described in U.S. Pat. No. 4,554,101, amino acid residues are given the following hydrophilicity indices: arginine (+3.0); lysine (+3.0); aspartic acid (+3.0±1); glutamic acid (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). It is understood that an amino acid may be substituted with another amino acid which has a similar hydrophilicity index and can still provide a biological equivalent. For such an amino acid substitution, the hydrophilicity index is preferably within 2, more preferably ±1, and even more preferably ±0.5.

The term “conservative substitution” as used herein refers to amino acid substitution in which a substituted amino acid and a substituting amino acid have similar hydrophilicity indices or/and hydrophobicity indices. For example, the conservative substitution is carried out between amino acids having a hydrophilicity or hydrophobicity index of within ±2, preferably within ±1, and more preferably within ±0.5. Examples of the conservative substitution include, but are not limited to, substitutions within each of the following residue pairs: arginine and lysine; glutamic acid and aspartic acid; serine and threonine; glutamine and asparagine; and valine, leucine, and isoleucine, which are well known to those skilled in the art.

As used herein, the term “variant” refers to a substance, such as a polypeptide, polynucleotide, or the like, which differs partially from the original substance. Examples of such a variant include a substitution variant, an addition variant, a deletion variant, a truncated variant, an allelic variant, and the like. Examples of such a variant include, but are not limited to, a nucleotide or polypeptide having one or several substitutions, additions and/or deletions or a nucleotide or polypeptide having at least one substitution, addition and/or deletion. The term “allele” as used herein refers to a genetic variant located at a locus identical to a corresponding gene, where the two genes are distinguished from each other. Therefore, the term “allelic variants” as used herein refers to a variant which has an allelic relationship with a given gene. Such an allelic variant ordinarily has a sequence the same as or highly similar to that of the corresponding allele, and ordinarily has almost the same biological activity, though it rarely has different biological activity. The term “species homolog” or “homolog” as used herein refers to an amino acid or nucleotide which has homology with a given gene in a given species (preferably at least 60% homology, more preferably at least 80%, at least 85%, at least 90%, and at least 95% homology). A method for obtaining such a species homolog is clearly understood from the description of the present specification. The term “orthologs” (also called orthologous genes) refers to genes in different species derived from a common ancestry (due to speciation). For example, in the case of the hemoglobin gene family having multigene structure, human and mouse α-hemoglobin genes are orthologs, while the human α-hemoglobin gene and the human β-hemoglobin gene are paralogs (genes arising from gene duplication). Orthologs are useful for estimation of molecular phylogenetic trees. Usually, orthologs in different species may have a function similar to that of the original species. Therefore, orthologs of the present invention may be useful in the present invention.

As used herein, the term “conservative (or conservatively modified) variant” applies to both amino acid and nucleic acid sequences. With respect to particular nucleic acid sequences, conservatively modified variants refer to those nucleic acids which encode identical or essentially identical amino acid sequences. Because of the degeneracy of the genetic code, a large number of functionally identical nucleic acids encode any given protein. For example, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” which represent one species of conservatively modified variation. Every nucleic acid sequence herein which encodes a polypeptide also describes every possible silent variation of the nucleic acid. Those skilled in the art will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence. Preferably, such modification may be performed while avoiding substitution of cysteine which is an amino acid largely affecting the higher-order structure of a polypeptide. Examples of a method for such modification of a base sequence include cleavage using a restriction enzyme or the like; ligation or the like by treatment using DNA polymerase, Klenow fragments, DNA ligase, or the like; and a site specific base substitution method using synthesized oligonucleotides (specific-site directed mutagenesis; Mark Zoller and Michael Smith, Methods in Enzymology, 100, 468-500 (1983)). Modification can be performed using methods ordinarily used in the field of molecular biology. Preferably, it is herein understood that such a conservatively modified variant can be used as a polypeptide or polynucleotide of the present invention.

In order to prepare functionally equivalent polypeptides, amino acid additions, deletions, or modifications can be performed in addition to amino acid substitutions. Amino acid substitution(s) refers to the replacement of at least one amino acid of an original peptide with different amino acids, such as the replacement of 1 to 10 amino acids, preferably 1 to 5 amino acids, and more preferably 1 to 3 amino acids with different amino acids. Amino acid addition(s) refers to the addition of at least one amino acid to an original peptide chain, such as the addition of 1 to 10 amino acids, preferably 1 to 5 amino acids, and more preferably 1 to 3 amino acids to an original peptide chain. Amino acid deletion(s) refers to the deletion of at least one amino acid, such as the deletion of 1 to 10 amino acids, preferably 1 to 5 amino acids, and more preferably 1 to 3 amino acids. Amino acid modification includes, but is not limited to, amidation, carboxylation, sulfation, halogenation, truncation, lipidation, alkylation, glycosylation, phosphorylation, hydroxylation, acylation (e.g., acetylation), and the like. Amino acids to be substituted or added may be naturally-occurring or non-naturally-occurring amino acids, or amino acid analogs. Naturally-occurring amino acids are preferable.

As used herein, the term “peptide analog” or “peptide derivative” refers to a compound which is different from a peptide but has at least one chemical or biological function equivalent to the peptide. Therefore, a peptide analog includes one that has at least one amino acid analog or amino acid derivative addition or substitution with respect to the original peptide. A peptide analog has the above-described addition or substitution so that the function thereof is substantially the same as the function of the original peptide (e.g., a similar pKa value, a similar functional group, a similar binding manner to other molecules, a similar water-solubility, and the like). Such a peptide analog can be prepared using techniques well known in the art. Therefore, a peptide analog may be a polymer containing an amino acid analog.

Similarly, the term “polynucleotide analogs or nucleic acid analog” refers to a compound which is different from a polynucleotide or a nucleic acid but has at least one chemical function or biological function equivalent to that of a polynucleotide or a nucleic acid. Therefore, a polynucleotide analog or a nucleic acid analog includes one that has at least one nucleotide analog or nucleotide derivative addition or substitution with respect to the original peptide.

Nucleic acid molecules as used herein includes one in which a part of the sequence of the nucleic acid is deleted or is substituted with other base(s), or an additional nucleic acid sequence is inserted, as long as a polypeptide expressed by the nucleic acid has substantially the same activity as that of the naturally-occurring polypeptide, as described above. Alternatively, an additional nucleic acid may be linked to the 5′ terminus and/or 3′ terminus of the nucleic acid. The nucleic acid molecule may include one that is hybridizable to a gene encoding a polypeptide under stringent conditions and encodes a polypeptide having substantially the same function as that of that polypeptide. Such a gene is known in the art and can be used in the present invention.

The above-described nucleic acid can be obtained by a well-known PCR method, i.e., chemical synthesis. This method may be combined with, for example, site-specific mutagenesis, hybridization, or the like.

As used herein, the term “substitution, addition or deletion” for a polypeptide or a polynucleotide refers to the substitution, addition or deletion of an amino acid or its substitute, or a nucleotide or its substitute with respect to the original polypeptide or polynucleotide. This is achieved by techniques well known in the art, including a site-specific mutagenesis technique and the like. A polypeptide or a polynucleotide may have any number (>0) of substitutions, additions, or deletions. The number can be large as long as a variant having such a number of substitutions, additions or deletions maintains an intended function (e.g., the information transfer function of hormones and cytokines, etc.). For example, such a number may be one or several, and preferably within 20% or 10% of the full length, or no more than 100, no more than 50, no more than 25, or the like.

(General Techniques)

Molecular biological techniques, biochemical techniques, and microorganism techniques as used herein are well known in the art and commonly used, and are described in, for example, Sambrook J. et al. (1989), Molecular Cloning: A Laboratory Manual, Cold Spring Harbor and its 3rd Ed. (2001); Ausubel, F. M. (1987), Current Protocols in Molecular Biology, Greene Pub. Associates and Wiley-interscience: Ausubel, F. M. (1989), Short Protocols in Molecular Biology: A Compendium of Methods from Current Protocols in Molecular Biology, Greene Pub. Associates and Wiley-interscience; Innis, M. A. (1990), PCR Protocols: A Guide to Methods and Applications, Academic Press; Ausubel, F. M. (1992), Short Protocols in Molecular Biology: A Compendium of Methods from Current Protocols in Molecular Biology, Greene Pub. Associates; Ausubel, F. M. (1995), Short Protocols in Molecular Biology: A Compendium of Methods from Current Protocols in Molecular Biology, Greene Pub. Associates; Innis, M. A. et al. (1995), PCR Strategies, Academic Press; Ausubel, F. M. (1999), Short Protocols in Molecular Biology: A Compendium of Methods from Current Protocols in Molecular Biology, Wiley, and annual updates; Sninsky, J. J. et al. (1999), PCR Applications: Protocols for Functional Genomics, Academic Press; Special issue, Jikken Igaku [Experimental Medicine] “Idenshi Donyu & Hatsugenkaiseki Jikkenho [Experimental Method for Gene Introduction & Expression Analysis]”, Yodo-sha, 1997; and the like. Relevant portions (or possibly the entirety) of each of these publications are herein incorporated by reference.

DNA synthesis techniques and nucleic acid chemistry for preparing artificially synthesized genes are described in, for example, Gait, M. J. (1985), Oligonucleotide Synthesis: A Practical Approach, IRL Press; Gait, M. J. (1990), Oligonucleotide Synthesis: A Practical Approach, IRL Press; Eckstein, F. (1991), Oligonucleotides and Analogues: A Practical Approach, IRL Press; Adams, R. L. et al. (1992), The Biochemistry of the Nucleic Acids, Chapman & Hall; Shabarova, Z. et al. (1994), Advanced Organic Chemistry of Nucleic Acids, Weinheim; Blackburn, G. M. et al. (1996), Nucleic Acids in Chemistry and Biology, Oxford University Press; Hermanson, G. T. (1996), Bioconjugate Techniques, Academic Press; and the like, related portions of which are herein incorporated by reference.

### (Genetic Engineering)

When a gene is mentioned herein, the term “vector” or “recombinant vector” refers to a vector transferring a polynucleotide sequence of interest to a target cell. Such a vector is capable of self-replication or incorporation into a chromosome in a host cell (e.g., a prokaryotic cell, yeast, an animal cell, a plant cell, an insect cell, an individual animal, and an individual plant, etc.), and contains a promoter at a site suitable for transcription of a polynucleotide of the present invention. A vector suitable for cloning is referred to as a “cloning vector”. Such a cloning vector ordinarily contains a multiple cloning site containing a plurality of restriction sites. Restriction enzyme sites and multiple cloning sites as described above are well known in the art and can be used as appropriate by those skilled in the art depending on the purpose in accordance with publications described herein (e.g., Sambrook et al., supra).

As used herein, the term “expression vector” refers to a nucleic acid sequence comprising a structural gene and a promoter for regulating expression thereof, and in addition, various regulatory elements in a state that allows them to operate within host cells. The regulatory element may include, preferably, terminators, selectable markers such as drug-resistance genes, and enhancers.

Examples of a “recombinant vector” for prokaryotic cells include, but are not limited to, pcDNA3 (+), pBluescript-SK (+/−), pGEM-T, pEF-BOS, pEGFP, pHAT, pUC18, pFT-DEST™42GATEWAY (Invitrogen), and the like.

Examples of a “recombinant vector” for animal cells include, but are not limited to, pcDNAI/Amp, pcDNAI, pCDM8 (all commercially available from Funakoshi), pAGE107 [Japanese Laid-Open Publication No. 3-229 (Invitrogen), pAGE103 [J. Biochem., 101,1307 (1987)], pAMo, pAMoA [J. Biol. Chem., 268, 22782-22787 (1993)], a retrovirus expression vector based on a murine stem cell virus (MSCV), PEF-BOS, pEGFP, and the like.

As used herein, the term “terminator” refers to a sequence which is located downstream of a protein-encoding region of a gene and which is involved in the termination of transcription when DNA is transcribed into mRNA, and the addition of a poly-A sequence. It is known that a terminator contributes to the stability of mRNA, and has an influence on the amount of gene expression.

As used herein, the term “promoter” refers to a base sequence which determines the initiation site of transcription of a gene and is a DNA region which directly regulates the frequency of transcription. Transcription is started by RNA polymerase binding to a promoter. A promoter region is usually located within about 2 kbp upstream of the first exon of a putative protein coding region. Therefore, it is possible to estimate a promoter region by predicting a protein coding region in a genomic base sequence using DNA analysis software. A putative promoter region is usually located upstream of a structural gene, but depending on the structural gene, i.e., a putative promoter region may be located downstream of a structural gene. Preferably, a putative promoter region is located within about 2 kbp upstream of the translation initiation site of the first exon.

As used herein, the term “enhancer” refers to a sequence which is used so as to enhance the expression efficiency of a gene of interest. One or more enhancers may be used, or no enhancer may be used.

As used herein, the term “operably linked” indicates that a desired sequence is located such that expression (operation) thereof is under control of a transcription and translation regulatory sequence (e.g., a promoter, an enhancer, and the like) or a translation regulatory sequence. In order for a promoter to be operably linked to a gene, typically, the promoter is located immediately upstream of the gene. A promoter is not necessarily adjacent to a structural gene.

Any technique may be used herein for introduction of a nucleic acid molecule into cells, including, for example, transformation, transduction, transfection, and the like. Such a nucleic acid molecule introduction technique is well known in the art and commonly used, and is described in, for example, Ausubel F. A. et al., editors, (1988), Current Protocols in Molecular Biology, Wiley, New York, N.Y.; Sambrook J. et al. (1987) Molecular Cloning: A Laboratory Manual, 2nd Ed. and its 3rd Ed., Cold Spring Harbor Laboratory Press, Cold Spring Harbor, N.Y.; Special issue, Jikken Igaku [Experimental Medicine] “Experimental Method for Gene Introduction & Expression Analysis”, Yodo-sha, 1997; and the like. Gene introduction can be confirmed by methods as described herein, such as Northern blotting analysis and Western blotting analysis, or other well-known, common techniques.

Any of the above-described methods for introducing DNA into cells can be used as a vector introduction method, including, for example, transfection, transduction, transformation, and the like (e.g., a calcium phosphate method, a liposome method, a DEAE dextran method, an electroporation method, a particle gun (gene gun) method, and the like).

As used herein, the term “transformant” refers to the whole or a part of an organism, such as a cell, which is produced by transformation. Examples of a transformant include a prokaryotic cell, yeast, an animal cell, a plant cell, an insect cell, and the like. Transformants may be referred to as transformed cells, transformed tissue, transformed hosts, or the like, depending on the subject. A cell used herein may be a transformant.

When a prokaryotic cell is used herein for genetic operations or the like, the prokaryotic cell may be of, for example, genus Escherichia, genus Serratia, genus Bacillus, genus Brevibacterium, genus Corynebacterium, genus Microbacterium, genus Pseudomonas, or the like. Specifically, the prokaryotic cell is, for example, Escherichia coli XL1-Blue, Escherichia coli XL2-Blue, Escherichia coli DH1, or the like.

Examples of an animal cell as used herein include a mouse myeloma cell, a rat myeloma cell, a mouse hybridoma cell, a Chinese hamster ovary (CHO) cell, a baby hamster kidney (BHK) cell, an African green monkey kidney cell, a human leukemic cell, HBT5637 (Japanese Laid-Open Publication No. 63-299), a human colon-cancer cell line, and the like. The mouse myeloma cell includes ps20, NSO, and the like. The rat myeloma cell includes YB2/0 and the like. A human embryo kidney cell includes HEK293 (ATCC:CRL-1573) and the like. The human leukemic cell includes BALL-1 and the like. The African green monkey kidney cell includes COS-1, COS-7, and the like. The human colon cancer cell line includes, but is not limited to, HCT-15, human neuroblastoma SK-N-SH, SK-N-SH-5Y, murine neuroblastoma Neuro2A, and the like.

Any method for introduction of DNA can be used herein as a method for introduction of a recombinant vector, including, for example, a calcium chloride method, an electroporation method (Methods. Enzymol., 194, 182 (1990)), a lipofection method, a spheroplast method (Proc. Natl. Acad. Sci. USA, 84, 1929 (1978)), a lithium acetate method (J. Bacteriol., 153,163 (1983)), a method described in Proc. Natl. Acad. Sci. USA, 75, 1929 (1978), and the like.

A retrovirus infection method as used herein is well known in the art as described in, for example, Current Protocols in Molecular Biology (supra) (particularly, Units 9.9-9.14), and the like. Specifically, for example, embryonic stem cells are trypsinized into a single-cell suspension, followed by co-culture with the culture supernatant of virus-producing cells (packaging cell lines) for 1-2 hours, thereby obtaining a sufficient amount of infected cells.

The transient expression of Cre enzyme, DNA mapping on a chromosome, and the like, which are used herein in a method for removing a genome, a gene locus, or the like, are well known in the art, as described in Kenichi Matsubara and Hiroshi Yoshikawa, editors, Saibo-Kogaku [Cell Engineering], special issue, “Experiment Protocol Series “FISH Experiment Protocol From Human Genome Analysis to Chromosome/Gene diagnosis”, Shujun-sha (Tokyo), and the like.

Gene expression (e.g., mRNA expression, polypeptide expression) may be “detected” or “quantified” by an appropriate method, including mRNA measurement and immunological measurement methods. Examples of the molecular biological measurement method include a Northern blotting method, a dot blotting method, a PCR method, and the like. Examples of the immunological measurement method include an ELISA method, an RIA method, a fluorescent antibody method, a Western blotting method, an immunohistological staining method, and the like, where a microtiter plate may be used. Examples of a quantification method include an ELISA method, an RIA method, and the like. A gene analysis method using an array (e.g., a DNA array, a protein array, etc.) may be used. The DNA array is widely reviewed in Saibo-Kogaku [Cell Engineering], special issue, “DNA Microarray and Up-to-date PCR Methods”, edited by Shujun-sha. The protein array is described in detail in Nat. Genet. 2002 December; 32 Suppl: 526-32. Examples of a method for analyzing gene expression include, but are not limited to, an RT-PCR method, a RACE method, an SSCP method, an immunoprecipitation method, a two-hybrid system, an in vitro translation method, and the like in addition to the above-described techniques. Other analysis methods are described in, for example, “Genome Analysis Experimental Method, Yusuke Nakamura's Labo-Manual, edited by Yusuke Nakamura, Yodo-sha (2002), and the like. All of the above-described publications are herein incorporated by reference.

As used herein, the term “amount of expression” refers to the amount of a polypeptide or mRNA expressed in a subject cell. The amount of expression includes the amount of expression at the protein level of a polypeptide of the present invention evaluated by any appropriate method using an antibody of the present invention, including immunological measurement methods (e.g., an ELISA method, an RIA method, a fluorescent antibody method, a Western blotting method, an immunohistological staining method, and the like, or the amount of expression at the mRNA level of a polypeptide of the present invention evaluated by any appropriate method, including molecular biological measurement methods (e.g., a Northern blotting method, a dot blotting method, a PCR method, and the like). The term “change in the amount of expression” indicates that an increase or decrease in the amount of expression at the protein or mRNA level of a polypeptide of the present invention evaluated by an appropriate method including the above-described immunological measurement method or molecular biological measurement method.

(Polypeptide Production Method)

A transformant derived from a microorganism, an animal cell, or the like, which possesses a recombinant vector into which DNA encoding a polypeptide of the present invention is incorporated, is cultured according to an ordinary culture method. The polypeptide of the present invention is produced and accumulated. The polypeptide of the present invention is collected from the culture, thereby making it possible to produce the polypeptide of the present invention.

The transformant of the present invention can be cultured on a culture medium according to an ordinary method for use in culturing host cells. A culture medium for a transformant obtained from a prokaryote (e.g., E. coli) or a eukaryote (e.g., yeast) as a host may be either a naturally-occurring culture medium or a synthetic culture medium as long as the medium contains a carbon source, a nitrogen source, inorganic salts, and the like which an organism of the present invention can assimilate and the medium allows efficient culture of the transformant.

The carbon source includes any one that can be assimilated by the organism, such as carbohydrates (e.g., glucose, fructose, sucrose, molasses containing these sugars, starch, starch hydrolysate, and the like), organic acids (e.g., acetic acid, propionic acid, and the like), alcohols (e.g., ethanol, propanol, and the like), and the like.

The nitrogen source includes ammonium salts of inorganic or organic acids (e.g., ammonia, ammonium chloride, ammonium sulfate, ammonium acetate, ammonium phosphate, and the like), and other nitrogen-containing substances (e.g., peptone, meat extract, yeast extract, corn steep liquor, casein hydrolysate, soybean cake, and soybean cake hydrolysate, various fermentation bacteria and digestion products thereof), and the like.

Salts of inorganic acids, such as potassium (I) phosphate, potassium (II) phosphate, magnesium phosphate, sodium chloride, iron (I) sulfate, manganese sulfate, copper sulfate, calcium carbonate, and the like, can be used. Culture is performed under aerobic conditions for shaking culture, deep aeration agitation culture, or the like.

Culture temperature is preferably 15 to 40° C., culture time is ordinarily 5 hours to 7 days. The pH of culture medium is maintained at 3.0 to 9.0. The adjustment of pH is carried out using inorganic or organic acid, alkali solution, urea, calcium carbonate, ammonia, or the like. An antibiotic, such as ampicillin, tetracycline, or the like, may be optionally added to culture medium during cultivation.

When culturing a microorganism which has been transformed using an expression vector containing an inducible promoter, the culture medium may be optionally supplemented with an inducer. For example, when a microorganism, which has been transformed using an expression vector containing a lac promoter, is cultured, isopropyl-β-D-thiogalactopyranoside or the like may be added to the culture medium. When a microorganism, which has been transformed using an expression vector containing a trp promoter, is cultured, indole acrylic acid or the like may be added to the culture medium. A cell or an organ into which a gene has been introduced can be cultured in a large volume using a jar fermenter. Examples of a medium for culture include, but are not limited to, commonly used Murashige and Skoog (MS) medium, White medium, or these media supplemented with plant hormones, such as auxin and cytokinins.

For example, when an animal cell is used, a culture medium of the present invention for culturing the cell includes a commonly used RPMI1640 culture medium (The Journal of the American Medical Association, 199, 519 (1967)), Eagle's MEM culture medium (Science, 122,501 (1952)), DMEM culture medium (Virology, 8, 396 (1959)), 199 culture medium (Proceedings of the Society for the Biological Medicine, 73, 1 (1950)) or these culture media supplemented with fetal bovine serum or the like.

Culture is normally carried out for 1 to 7 days under conditions such as pH 6 to 8, 25 to 40° C., and 5% CO₂. An antibiotic, such as kanamycin, penicillin, streptomycin, or the like may be optionally added to the culture medium during cultivation.

A polypeptide of the present invention can be isolated or purified from a culture of a transformant, which has been transformed with a nucleic acid sequence encoding the polypeptide, using an ordinary method for isolating or purifying enzymes, which are well known and commonly used in the art. For example, when a polypeptide of the present invention is secreted outside a transformant for producing the polypeptide, the culture is subjected to centrifugation or the like to obtain a soluble fraction. A purified specimen can be obtained from the soluble fraction by a technique, such as solvent extraction, salting-out/desalting with ammonium sulfate or the like, precipitation with organic solvent, anion exchange chromatography with a resin (e.g., diethylaminoethyl (DEAE)-Sepharose, DIAION HPA-75 (Mitsubishi Kasei Corporation), etc.), cation exchange chromatography with a resin (e.g., S-Sepharose FF (Pharmacia), etc.), hydrophobic chromatography with a resin (e.g., buthylsepharose, phenylsepharose, etc.), gel filtration with a molecular sieve, affinity chromatography, chromatofocusing, electrophoresis (e.g., isoelectric focusing electrophoresis, etc.).

When a polypeptide of the present invention is accumulated in a dissolved form within a transformant cell for producing the polypeptide, the culture is subjected to centrifugation to collect cells in the culture. The cells are washed, followed by pulverization of the cells using an ultrasonic pulverizer, a French press, MANTON GAULIN homogenizer, Dinomil, or the like, to obtain a cell-free extract solution. A purified specimen can be obtained from a supernatant obtained by centrifuging the cell-free extract solution or by a technique, such as solvent extraction, salting-out/desalting with ammonium sulfate or the like, precipitation with organic solvent, anion exchange chromatography with a resin (e.g., diethylaminoethyl (DEAE)-Sepharose, DIAION HPA-75 (Mitsubishi Kasei Corporation), etc.), cation exchange chromatography with a resin (e.g., S-Sepharose FF (Pharmacia), etc.), hydrophobic chromatography with a resin (e.g., buthylsepharose, phenylsepharose, etc.), gel filtration with a molecular sieve, affinity chromatography, chromatofocusing, electrophoresis (e.g., isoelectric focusing electrophoresis, etc.).

When the polypeptide of the present invention has been expressed and formed insoluble bodies within cells, the cells are harvested, pulverized, and centrifuged. From the resulting precipitate fraction, the polypeptide of the present invention is collected using a commonly used method. The insoluble polypeptide is solubilized using a polypeptide denaturant. The resulting solubilized solution is diluted or dialyzed into a denaturant-free solution or a dilute solution, where the concentration of the polypeptide denaturant is too low to denature the polypeptide. The polypeptide of the present invention is allowed to form a normal three-dimensional structure, and the purified specimen is obtained by isolation and purification as described above.

Purification can be carried out in accordance with a commonly used protein purification method (J. Evan. Sadler et al.: Methods in Enzymology, 83, 458). Alternatively, the polypeptide of the present invention can be fused with other proteins to produce a fusion protein, and the fusion protein can be purified using affinity chromatography using a substance having affinity for the fusion protein (Akio Yamakawa, Experimental Medicine, 13, 469-474 (1995)). For example, in accordance with a method described in Lowe et al., Proc. Natl. Acad. Sci., USA, 86, 8227-8231 (1989), Genes Develop., 4, 1288 (1990)), a fusion protein of the polypeptide of the present invention with protein A is produced, followed by purification with affinity chromatography using immunoglobulin G.

A fusion protein of the polypeptide of the present invention with a FLAG peptide is produced, followed by purification with affinity chromatography using anti-FLAG antibodies (Proc. Natl. Acad. Sci., USA, 86, 8227 (1989), Genes Develop., 4, 1288 (1990)).

The polypeptide of the present invention can be purified with affinity chromatography using antibodies which bind to the polypeptide. The polypeptide of the present invention can be produced using an in vitro transcription/translation system in accordance with a known method (J. Biomolecular NMR, 6, 129-134; Science, 242, 1162-1164; J. Biochem., 110, 166-168 (1991)).

Based on the amino acid information of a polypeptide as obtained above, the polypeptide can also be produced by a chemical synthesis method, such as the Fmoc method (fluorenylmethyloxycarbonyl method), the tBoc method (t-buthyloxycarbonyl method), or the like. The peptide can be chemically synthesized using a peptide synthesizer (manufactured by Advanced ChemTech, Applied Biosystems, Pharmacia Biotech, Protein Technology instrument, Synthecell-Vega, PerSeptive, Shimazu, or the like).

The structure of the purified polypeptide of the present invention can be carried out by methods commonly used in protein chemistry (see, for example, Hisashi Hirano. “Protein Structure Analysis for Gene Cloning”, published by Tokyo Kagaku Dojin, 1993). The physiological activity of a polypeptide of the present invention can be measured in accordance with a known measurement method.

(Method for Producing Mutant Polypeptide)

Amino acid deletion, substitution or addition of the polypeptide of the present invention can be carried out by a site-specific mutagenesis method which is a well known technique. One or several amino acid deletions, substitutions or additions can be carried out in accordance with methods described in Molecular Cloning, A Laboratory Manual, Second Edition, Cold Spring Harbor Laboratory Press (1989); Current Protocols in Molecular Biology, Supplement 1 to 38, John Wiley &Sons (1987-1997); Nucleic Acids Research, 10, 6487 (1982); Proc. Natl. Acad. Sci., USA, 79, 6409 (1982); Gene, 34, 315 (1985); Nucleic Acids Research, 13, 4431 (1985); Proc. Natl. Acad. Sci. USA, 82, 488 (1985); Proc. Natl. Acad. Sci., USA, 81, 5662 (1984); Science, 224, 1431 (1984); PCT WO85/00817 (1985); Nature, 316, 601 (1985) and the like.

(Immunochemistry)

Preparation of Antibodies which Recognize the polypeptide of the present invention are also well known in the art. For example, preparation of polyclonal antibodies can be carried out by administering a purified specimen of the whole or a partial fragment of an obtained polypeptide or a peptide having a portion of the amino acid sequence of the protein of the present invention, as an antigen, to an animal.

To produce antibodies, a rabbit, a goat, a rat, a mouse, a hamster, or the like can be used as an animal to which an antigen is administered. The dose of the antigen is preferably 50 to 100 μg per animal. When a peptide is used as an antigen, the peptide is preferably coupled via covalent bond to a carrier protein, such as keyhole limpet haemocyanin, bovine thyroglobulin, or the like. A peptide used as an antigen can be synthesized using a peptide synthesizer. The antigen is administered every 1 to 2 weeks after a first administration a total 3 to 10 times. Blood is collected from the venous plexus of eye grounds 3 to 7 days after each administration, and the reactivity of the serum with the antigen, which has been used for immunization, is determined by an enzyme immunoassay (Enzyme immunoassay (ELISA): published by Igaku-syoin 1976; Antibodies-A Laboratory Manual, Cold Spring Harbor Laboratory (1988); and the like).

Serum is obtained from a non-human mammal whose serum exhibits a sufficient antibody titer to an antigen. From the serum, polyclonal antibodies can be isolated and purified using well known techniques. Production of monoclonal antibodies is also well known in the art. In order to prepare antibody secreting cells, a rat whose serum exhibits a sufficient antibody titer for fragments of a polypeptide of the present invention which has been used for immunization, is used as a source for antibody secreting cells, which are fused with myeloma cells to prepare hybridomas. Thereafter, a hybridoma specifically reacting with the fragments of the polypeptide of the present invention is selected using enzyme immunoassays. A monoclonal antibody secreted by the thus-obtained hybridoma can be used for various purposes.

Such an antibody can be used for an immunological method of detecting the polypeptide of the present invention, for example. Examples of an immunological method of detecting the polypeptide of the present invention using the antibody of the present invention include an ELISA method using microtiter plates, a fluorescent antibody method, a Western blotting method, an immunohistological method, and the like.

Further, the antibody of the present invention can be used for immunological methods for quantifying the amount of the polypeptide of the present invention. Examples of the immunological methods for quantifying the amount of the polypeptide of the present invention include a sandwich ELISA method using two monoclonal antibodies for different epitopes of the polypeptide of the present invention, which react with the polypeptide of the present invention; a radioimmunoassay using the polypeptide of the present invention labeled with a radioactive isotope, such as 126, or the like, and antibodies which recognize the polypeptide of the present invention; and the like.

Methods for quantifying mRNA for the polypeptide of the present invention are well known in the art. For example, the above-described oligonucleotides prepared from the polynucleotide or DNA of the present invention can be used to quantify the level of expression of DNA encoding the polypeptide of the present invention based on the mRNA level using Northern hybridization or PCR. Such a technique is well known in the art and is described in literature described herein.

The polynucleotides may be obtained, and the nucleotide sequence of the polynucleotides determined, by any method known in the art. For example, if the nucleotide sequence of an antibody is known, a polynucleotide encoding the antibody may be assembled from chemically synthesized oligonucleotides (e.g., as described in Kutmeier et al., BioTechniques, 17: 242 (1994)), which, briefly, involves the synthesis of overlapping oligonucleotides containing portions of the sequence encoding the antibody, annealing and ligation of those oligonucleotides, and then amplification of the ligated oligonucleotides by PCR.

Alternatively, a polynucleotide encoding an antibody can be produced from a nucleic acid from a suitable source. If a clone containing a nucleic acid encoding a particular antibody is not available, but the sequence of the antibody molecule is known, a nucleic acid encoding the immunoglobulin may be obtained from a suitable source (e.g., an antibody cDNA library, or a cDNA library generated from any tissue or cells expressing the antibody (e.g., hybridoma cells selected to express an antibody of the present invention), or nucleic acids (preferably poly-A+RNA) isolated therefrom) by PCR amplification using synthetic primers hybridizable to the 3′ and 5′ ends of the sequence or by cloning using an oligonucleotide probe specific for the particular gene sequence to identify, for example, a cDNA clone from a cDNA library that encodes the antibody. Amplified nucleic acids produced by PCR may be cloned into replicable cloning vectors using any method well known in the art.

Once the nucleotide sequence and corresponding amino acid sequence of an antibody is determined, the nucleotide sequence of the antibody may be manipulated using methods well known in the art for the manipulation of nucleotide sequences (e.g., recombinant DNA techniques, site directed mutagenesis, PCR, etc. (see, for example, the techniques described in Sambrook et al., 1990, Molecular Cloning, A Laboratory Manual, 2nd Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entirety), to produce antibodies having a different amino acid sequence, for example, to create amino acid substitutions, deletions, and/or insertions.

In a specific embodiment, the amino acid sequence of heavy and/or light chain variable domains may be inspected to identify the sequences of the complementarity determining regions (CDRs) by methods that are well know in the art (e.g., by comparison to known amino acid sequences of other heavy and light chain variable regions to determine the regions of sequence hypervariability). Using routine recombinant DNA techniques, one or more of the CDRs may be inserted within framework regions (e.g., into human framework regions to humanize a non-human antibody) as described above. The framework regions may be naturally occurring or consensus framework regions, and preferably human framework regions (see, e.g., Chothia et al., J. Mol. Biol. 278: 457-479 (1998) for a listing of human framework regions). Preferably, the polynucleotide generated by the combination of the framework regions and CDRs encodes an antibody that specifically binds a polypeptide of the present invention. Preferably, as discussed above, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and are within the skill of one skilled in the art.

In addition, techniques developed for the production of “chimeric antibodies” (Morrison et al., 1984, Proc. Natl. Acad. Sci. 81:851-855: Neuberger et al., 1984, Nature 312:604-608; Takeda et al., 1985, Nature 314: 452-454) by splicing genes from a mouse antibody molecule of appropriate antigen specificity together with genes from a human antibody molecule of appropriate biological activity can be used. As described above, a chimeric antibody is a molecule in which different portions are derived from different animal species. Such a molecule has a variable region derived from a murine mAb and a human immunoglobulin constant region (e.g., humanized antibodies).

Known techniques described for the production of single chain antibodies (U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., Science 242:1038-1041 (1988)).

(Methods of Producing Antibodies)

The antibodies of the present invention can be produced by any method known in the art for the synthesis of antibodies, by chemical synthesis, or preferably, by recombinant expression techniques.

Recombinant expression of an antibody of the present invention, or fragment, derivative or analog thereof (e.g., a heavy or light chain of an antibody of the present invention) requires construction of an expression vector containing a polynucleotide that encodes the antibody. Once a polynucleotide encoding an antibody molecule or a heavy or light chain of an antibody, or portion thereof (preferably containing the heavy or light chain variable domain), of the present invention has been obtained, a vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art may be used to construct expression vectors containing antibody coding sequences and appropriate transcriptional and translational control signals. These methods include, for example, in vitro recombinant DNA techniques, synthetic techniques, and in vivo genetic recombination. The present invention, thus, provides replicable vectors comprising a nucleotide sequence encoding an antibody molecule of the present invention, or a heavy or light chain thereof, or a heavy or light chain variable domain, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy or light chain.

The expression vector is transferred to a host cell by conventional techniques and the transfected cells are then cultured by conventional techniques to produce an antibody of the present invention. Thus, the present invention includes host cells containing a polynucleotide encoding an antibody of the present invention, or a heavy or light chain thereof, operably linked to a heterologous promoter. In preferred embodiments for the expression of double-chained antibodies, vectors encoding both the heavy and light chains may be co-expressed in the host cell for expression of the entire immunoglobulin molecule, as detailed below.

(Screening)

As used herein, the term “screening” refers to selection of a target, such as an organism, a substance, or the like, a given specific property of interest from a population containing a number of elements using a specific operation/evaluation method. For screening, an agent (e.g., an antibody), a polypeptide or a nucleic acid molecule of the present invention can be used. Screening may be performed using libraries obtained in vitro, in vivo, or the like (with a system using a real substance) or alternatively In silico (with a system using a computer). It will be understood that the present invention encompasses compounds having desired activity obtained by screening. The present invention is also intended to provide drugs which are produced by computer modeling based on the disclosures of the present invention.

(Diseases)

As used herein, the terms “axon” and “neurite” are used interchangeably, referring to a neuronal protrusion. Axons grow when they are stimulated. This is called axon outgrowth. To determine whether or not axon outgrowth occurs, the length of the longest axon is measured before and after stimulation, and the ratio of the lengths is confirmed.

As used herein, the term “fasciculation” indicates that a plurality of axons are formed into a fascicle. The presence or absence of fasciculation can be determined by observing the formation of a fiber using an actin immunological staining technique and an electron microscope.

As used herein, the term “level of axon outgrowth and/or fasciculation” can be described and compared based on the results of the above-described determination assays. Such a level can be confirmed by any techniques known in the art which can be used to measure the level of axon outgrowth and/or fasciculation in addition to the above-described assay.

As used herein, term “condition associated with a level of axon outgrowth and/or fasciculation” refers to a condition in an organism associated with a level of axon outgrowth and/or fasciculation. Therefore, it is intended that such a condition encompasses any conditions affected by the level of axon outgrowth and/or fasciculation no matter whether or not the organism has a disorder or disease (including a healthy condition). Therefore, such a condition may include a psychiatric condition, for example.

As used herein, the term “disorder and/or disease associated with a level of axon outgrowth and/or fasciculation” refers to a disorder and/or disease of an organism associated with a level of axon outgrowth and/or fasciculation, including a condition having a psychological, physiological or anatomical loss or abnormality of structure or function and/or a condition having a symptom or syndrome which can be clearly indicated or a consistent anatomical change. Examples of such a disease include, but are not limited to, schizophrenia, mental retardation, depression, epilepsy, and the like.

(Gene Therapy)

In a specific embodiment of the present invention, a nucleic acid comprising a sequence encoding a nucleic acid sequence of a normal gene of the present invention, or an antibody or a functional derivative thereof, is administered for the purpose of gene therapy for treatment, inhibition, or prophylaxis of a disease or a disorder associated with abnormal expression and/or activity of a polypeptide of the present invention. Gene therapy means that subjects are treated by administering an expressed or expressable nucleic acid thereto. In this embodiment of the present invention, a protein encoded by a nucleic acid is produced and the protein mediates a therapeutic effect.

Any technique available in the art for gene therapy may be employed in the present invention. Illustrative techniques are described as follows.

Gene therapy techniques are generally reviewed in, for example, Goldspiel et al., Clinical Pharmacy 12: 488-505 (1993); Wu and Wu, Biotherapy 3: 87-95 (1991); Tolstoshev, Ann. Rev. Pharmacol. Toxicol., 32: 573-596 (1993); Mulligan, Science 260: 926-932 (1993); and Morgan and Anderson, Ann. Rev. Biochem., 62: 191-217 (1993); May, TIBTECH 11(5): 155-215 (1993). Recombinant DNA techniques generally known, which are generally used in gene therapy, are described in, for example, Ausubel et al. (ed.), Current Protocols in Molecular Biology, John Wiley & Sons, NY (1993); and Kriegler, Gene Transfer and Expression, A Laboratory Manual, Stockton Press, NY (1990).

(Demonstration of Therapeutic Activity or Prophylactic Activity)

The compounds or pharmaceutical compositions of the present invention are preferably tested in vitro, and then in vivo for the desired therapeutic or prophylactic activity, prior to use in humans. For example, in vitro assays to demonstrate therapeutic or prophylactic utility of a compound or pharmaceutical composition include, the effect of a compound on a cell line or a patient tissue sample. The effect of the compound or composition on the cell line and/or tissue sample can be determined utilizing techniques known to those of skill in the art (including, but not limited to, cell lysis assays). In accordance with the present invention, in vitro assays which can be used to determine whether administration of a specific compound is indicated, include in vitro cell culture assays in which a patient tissue sample is grown in culture, and exposed to or otherwise administered a compound, and the effect of such compound upon the tissue sample is observed.

(Therapeutic/Prophylactic Administration and Composition)

The present invention provides methods of treatment, inhibition and prophylaxis by administration to a subject of an effective amount of a compound or pharmaceutical composition of the present invention. In a preferred aspect, the compound is substantially purified (e.g., substantially free from substances that limit its effect or produce undesired side-effects).

Animals targeted by the present invention include any organism as long as it has a nervous system or a system similar thereto (e.g., vertebrates and invertebrate animals). Preferably, the animal is a vertebrate (e.g., Myxiniformes, Petronyzoniformes, Chondrichthyes, Osteichthyes, amphibian, reptilian, avian, mammalian, etc.), more preferably mammalian (e.g., monotremata, marsupialia, edentate, dermoptera, chiroptera, carnivore, insectivore, proboscidea, perissodactyla, artiodactyla, tubulidentata, pholidota, sirenia, cetacean, primates, rodentia, lagomorpha, etc.). Illustrative examples of a subject include, but are not limited to, animals, such as cattle, pigs, horses, chickens, cats, dogs, and the like. More preferably, Primates (e.g., chimpanzee, Japanese monkey, human, etc.) are used. Most preferably, a human is used.

When a nucleic acid molecule or polypeptide of the present invention is used as a medicament, the medicament may further comprise a pharmaceutically acceptable carrier. Any pharmaceutically acceptable carrier known in the art may be used in the medicament of the present invention.

Examples of a pharmaceutically acceptable carrier or a suitable formulation include, but are not limited to, antioxidants, preservatives, colorants, flavoring agents, diluents, emulsifiers, suspending agents, solvents, fillers, bulky agents, buffers, delivery vehicles, and/or pharmaceutical adjuvants. Representatively, a medicament of the present invention is administered in the form of a composition comprising an isolated pluripotent stem cell, or a variant or derivative thereof, with at least one physiologically acceptable carrier, excipient or diluent. For example, an appropriate vehicle may be an injection solution, physiological solution, or artificial cerebrospinal fluid, which can be supplemented with other substances which are commonly used for compositions for parenteral delivery.

Acceptable carriers, excipients or stabilizers used herein preferably are non-toxic to recipients and are preferably inert at the dosages and concentrations employed, and preferably include phosphate, citrate, or other organic acids: ascorbic acid, α-tocopherol; low molecular weight polypeptides; proteins (e.g., serum albumin, gelatin, or immunoglobulins); hydrophilic polymers (e.g., polyvinylpyrrolidone); amino acids (e.g., glycine, glutamine, asparagine, arginine or lysine); monosaccharides, disaccharides, and other carbohydrates (glucose, mannose, or dextrins); chelating agents (e.g., EDTA); sugar alcohols (e.g., mannitol or sorbitol); salt-forming counterions (e.g., sodium); and/or non-ionic surfactants (e.g., Tween, pluronics or polyethylene glycol (PEG)).

Examples of appropriate carriers include neutral buffered saline or saline mixed with serum albumin. Preferably, the product is formulated as a lyophilizate using appropriate excipients (e.g., sucrose). Other standard carriers, diluents, and excipients may be included as desired. Other exemplary compositions comprise Tris buffer of about pH 7.0-8.5, or acetate buffer of about pH 4.0-5.5, which may further include sorbitol or a suitable substitute therefor.

The medicament of the present invention may be administered orally or parenterally. Alternatively, the medicament of the present invention may be administered intravenously or subcutaneously. When systemically administered, the medicament for use in the present invention may be in the form of a pyrogen-free, pharmaceutically acceptable aqueous solution. The preparation of such pharmaceutically acceptable compositions, with due regard to pH, isotonicity, stability and the like, is within the skill of one skilled the art. Administration methods may be herein oral, parenteral administration (e.g., intravenous, intramuscular, subcutaneous, intradermal, to mucosa, intrarectal, vaginal, topical to an affected site, to the skin, etc.). A prescription for such administration may be provided in any formulation form. Such a formulation form includes liquid formulations, injections, sustained preparations, and the like.

The medicament of the present invention may be prepared for storage by mixing an active ingredient having the desired degree of purity with optional physiologically acceptable carriers, excipients, or stabilizers (Japanese Pharmacopeia ver. 14, or a supplement thereto or the latest version; Remington's Pharmaceutical Sciences, 18th Edition, A. R. Gennaro, ed., Mack Publishing Company, 1990; and the like), in the form of lyophilized cake or aqueous solutions.

The amount of an active ingredient used in the treatment method of the present invention can be easily determined by those skilled in the art with reference to the purpose of use, a target disease (type, severity, and the like), the patient's age, weight, sex, and case history, the form or type of the active ingredient, and the like. The frequency of the treatment by the method of the present invention applied to a subject (or patient) is also determined by those skilled in the art with respect to the purpose of use, target disease (type, severity, and the like), the patient's age, weight, sex, and case history, the progression of therapy, and the like. Examples of the frequency include once per day to several months (e.g., once per week to once per month). Preferably, administration is performed once per week to month with reference to the progression.

As used herein, the term “instructions” describe a method of administering a medicament, a method for diagnosis, or the like of the present invention for persons who administer, or are administered, the medicament or the like or persons who diagnose or are diagnosed (e.g, physicians, patients, and the like). The instructions describe a statement indicating an appropriate method for administering a diagnostic, a medicament, or the like of the present invention. The instructions are prepared in accordance with a format defined by an authority of a country in which the present invention is practiced (e.g., Health, Labor and Welfare Ministry in Japan, Food and Drug Administration (FDA) in the U.S., and the like), explicitly describing that the instructions are approved by the authority. The instructions are so-called package insert and are typically provided in paper media. The instructions are not so limited and may be provided in the form of electronic media (e.g., web sites, electronic mails, and the like provided on the internet).

(Detailed Description of Disc1)

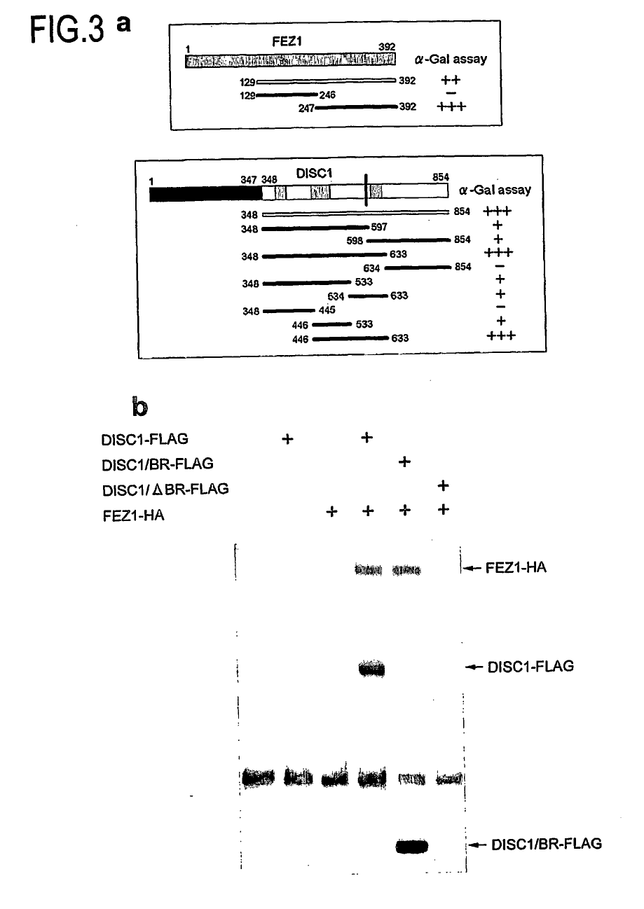

In the present study, the present inventors identified FEZ1 as an interacting partner of DISC1 by a yeast two-hybrid study. FEZ1 is a mammalian homologue of the Caenorhabditis elegans UNC-76 protein involved in axonal outgrowth and fasciculation (e.g., Hall A., Science, 1998, 279: 509-514; and Luo L., Nature Rev. Neurosci., 2000, 1:173-180). The interaction between DISC1 and FEZ1 was up-regulated in PC12 cells during neuronal differentiation. Moreover, neurite outgrowth was enhanced by the overexpression of DISC1, and inhibition of the interaction between DISC1 and FEZ1 disturbed this enhanced neurite outgrowth. These results suggest that DISC1 participates in neurite extension machinery through its interaction with FEZ1. It should be noted that a DISC1 truncated form lacking the C-terminus downstream of the translocation breakpoint, showed reduced potential for interaction with FEZ1 ((a) of FIG. 3), because production of the truncated DISC1 protein would be possible in translocation carriers (Millar J. K., Wilson-Annan J. C., Anderson S., Christie S., Taylor M. S., Semple C. A. M. et al., Hum. Mol. Genet., 2000, 9: 1415-1423).

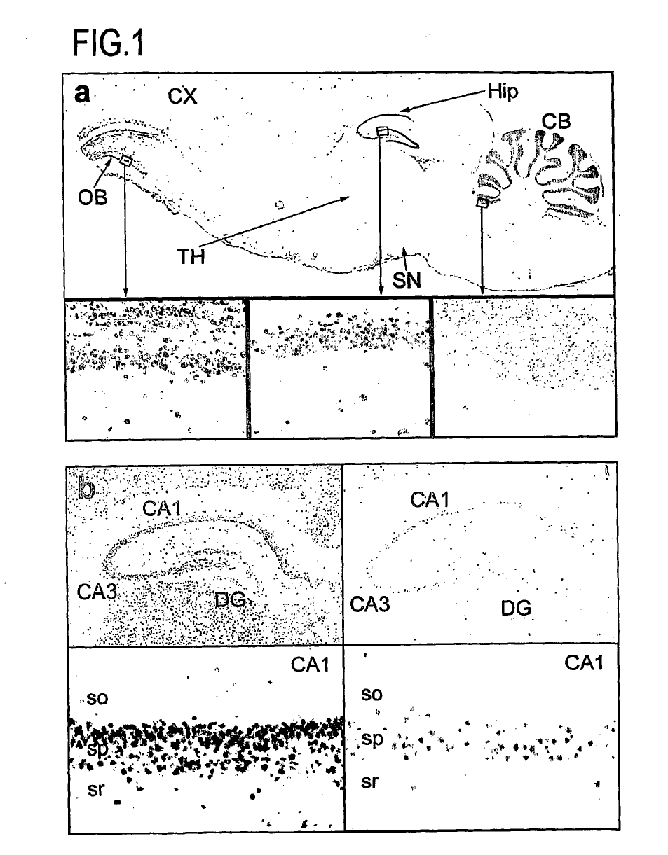

In a Scottish family, the translocation was not associated with any physical disorders (Blackwood D. H. R., Fordyce A., Walker M. T., St. Clair D. M., Porteous D. J., Muir W. J., Am. J. Hum. Genet., 2001, 69: 428-433). It is unclear why the disruption of DISC1 by translocation causes psychiatric diseases selectively regardless of the expression of DISC1 throughout the body (Millar J. K., Wilson-Annan J. C., Anderson S., Christie S., Taylor M. S., Semple C. A. M. et al., Hum. Mol. Genet., 2000, 9: 1415-1423). However, the restricted expression of interacting partner(s) to brain may well explain this selectivity. The expression of FEZ1 is highly specific to brain (Luo L., Nature Rev. Neurosci., 2000, 1:173-180), and the elevated level of FEZ1 expression is observed in neurons of rat brain at embryonic 18 day and postnatal 7 day (unpublished data). In the study of nematodes, severe defects in newly hatched unc-76 mutant larvae suggest the importance of UNC-76 in nervous system development (Hall A., Science, 1998, 279: 509-514). Combined with the enhanced DISC1 expression in rat brain at a developing stage ((b) of FIG. 1), these findings imply that FEZ1/DISC1 interaction plays a crucial role in the development of the mammalian nervous system.

Recent studies have provided reliable evidence that schizophrenia is a neurodevelopmental disorder (Sawa A., Snyder S. H., Science, 2002, 296: 692-695; Harrison P. J., Brain, 1999, 122: 593-624; and Heckers S., Konradi C., J. Neural. Transm., 2002, 109: 891-905). Cytoarchitectual change in the hippocampus has been noteworthy among the various neuropathological abnormalities reported in schizophrenia (Harrison P. J., Eastwood S. L., Hippocampus 2001, 11: 508-519; Ozeki Y., Tomoda T., Kleiderlein J., Kamiya A., Bord L., Fujii K. et al, Proc. Natl. Acad. Sci., January 2003; and Millar J. K., James R., Christie S., Taylor M. S., Devon R. S., Hogg G. et al., Abstract from Xth World Congress on Psychiatric Genetics 2002). Decreased neuronal size and alterations in presynaptic and dendritic markers suggest that abnormalities in the hippocampal neural circuitry result in schizophrenia (Millar J. K., James R., Christie S., Taylor M. S., Devon R. S., Hogg G. et al., Abstract from Xth World Congress on Psychiatric Genetics, 2002). In this regard, it is of note that the expression of DISC1 was abundant in hippocampal neurons especially at a developing stage ((a) and (b) of FIG. 1), suggesting the potential involvement of DISC1 in the formation of the hippocampal neural circuits. This raises the possibility that dysfunction of DISC1 may result in abnormal development of the nervous system, leading to susceptibility to psychiatric illnesses.

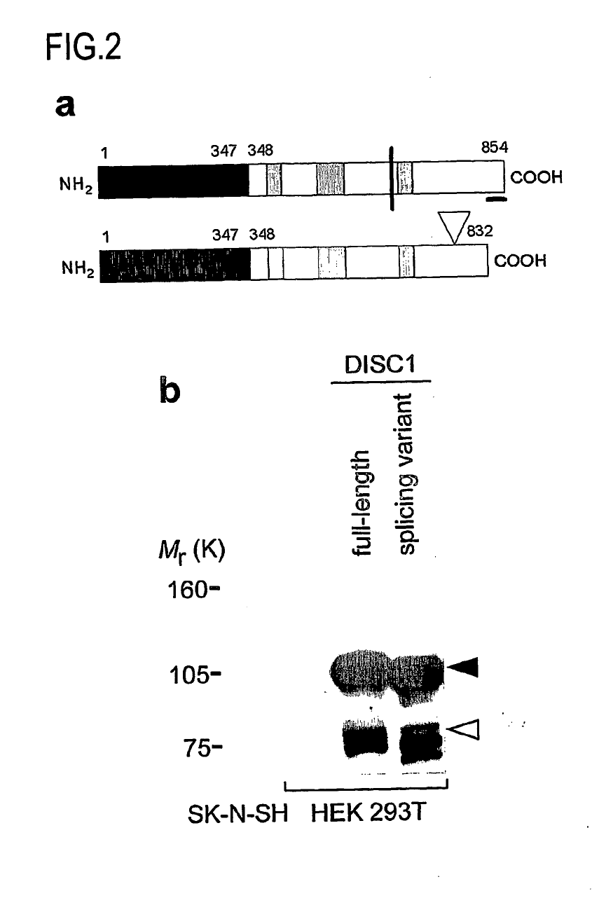

In a western blot analysis, an antibody raised against DISC1 detected at least two bands in lysates from human cell lines ((a) of FIG. 2). The band of 105 K fitted the expected size of DISC1. As an overexpressed 78-K protein was detected in the lysate from cells transiently transfected with full-length DISC1 cDNA, this smaller form of DISC1 was thought to arise from post-translational modification. Further examination as to origin of 78-K protein is needed. A recent study has reported the existence of two forms of DISC1 in rat brain and the up-regulated expression of the full-length form in developing stages, and suggested the importance of the full-length form with respect to the development of the nervous system (Kandpal G., Ma L., Acton P., Austin C. P., Morris J. A., Abstract from Society for Neuroscience 32nd Annual Meeting, 2002).

The entire pathway involving DISC1 and FEZ1 is still unclear, although these components were identified as cytoskeletal-associated proteins by our findings. The possible implications of DISC1 in the cytoskeleton are also predicted by the recent study (Kandpal G., Ma L., Acton P., Austin C. P., Morris J. A., Abstract from Society for Neuroscience 32nd Annual Meeting, 2002) and preliminary reports (Ramakers G. J. A., Trends Neurosci., 2002, 25: 191-199; and Chelly J., Mandel J. L., Nat. Rev. Genet., 2001, 2: 669-680). Rho GTPases, which are signaling proteins that orchestrate coordinated changes in the actin cytoskeleton essential for directed neurite outgrowth (e.g., Weinberger D. R., Arch. Gen. Psychiatry, 1987, 44: 660-669; and Lewis D. A., Levitt P., Annu. Rev. Neurosci., 2002, 25: 409-432), might be key molecules in further studies to delineate the biological role of the DISC1/FEZ1 complex. Interestingly, the regulation of the actin cytoskeleton in the developing nervous system is involved in the pathogenesis of mental retardation (Billuart P., Bienvenu T., Ronce N., des Portes V., Vinet M. C., Zemni R. et al., Nature, 1998, 392: 923-926). Seven genes have been identified to be responsible for X-linked mental retardation when mutated (Allen K. M., Gleeson J. G., Bagrodia S., Partington M. W., MacMillan J. C., Cerione R. A. et al., Nat. Genet., 1998, 20:25-30). Three of these genes encode oligophrenin-1 (Kutsche K., Yntema H., Brandt A., Jantke I., Nothwang H. G., Orth U. et al., Nat. Genet., 2000, 26: 247-250), DKA3 (Allen K. M., Gleeson J. G., Bagrodia S., Partington M. W., MacMillan J. C., Cerione R. A. et al., Nat. Genet., 1998, 20:25-30) and αPIX (Kutsche K., Yntema H., Brandt A., Jantke I., Nothwang H. G., Orth U. et al., Nat. Genet., 2000,26: 247-250). They interact directly with Rho GTPases. So far, the disruption of DISC1 by translocation has been found in one family from Scotland. Although dysfunction of DISC1 might account for the pathogenesis of a small subgroup of schizophrenia, elucidation of the pathophysiological role of DISC1 will provide a better understanding of the general etiology of schizophrenia.

KIAA0844 was also identified by a two-hybrid method using DISC1. Therefore, it was revealed that KIAA0844 plays a role in neurotransmission pathways. Therefore, KIAA0844 is also useful for a marker, kit and method for determining a level of axon outgrowth and/or fasciculation, or a condition, disorder or disease associated with the level, as well as DISC1 and FEZ1.

### DESCRIPTION OF PREFERRED EMBODIMENTS

Hereinafter, preferred embodiments of the present invention will be described. The following embodiments are provided for a better understanding of the present invention and the scope of the present invention should not be limited to the following description. It will be clearly appreciated by those skilled in the art that variations and modifications can be made without departing from the scope of the present invention with reference to the specification.

In one aspect, the present invention relates to an agent specifically interacting with a polynucleotide encoding DISC1 or a fragment thereof. Representatively, DISC1 comprises:

(a) a polynucleotide having abase sequence set forth in SEQ ID NO. 1 or a fragment thereof;

(b) a polynucleotide encoding a polypeptide consisting of an amino acid sequence set forth in SEQ ID NO. 2 or a fragment thereof;

(c) a polynucleotide encoding a variant polypeptide having an amino acid sequence set forth in SEQ ID NO. 2 or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 1;

(e) a polynucleotide encoding a species homolog of a polypeptide consisting of an amino acid sequence set forth in SEQ ID NO. 2;

(f) a polynucleotide hybridizable to any one of the polynucleotides of (a) to (e) under stringent conditions and encoding a polypeptide having biological activity: or

(g) a polynucleotide consisting of a base sequence having at least 70% identity to any one of the polynucleotides (a) to (e) or a complementary sequence thereof, and encoding a polypeptide having biological activity.

In one preferred embodiment, the number of substitutions, additions, and deletions in (c) is preferably limited, for example, 50 or less, 40 or less, 30 or less, 20 or less, 15 or less, 10 or less, 9 or less, 8 or less, 7 or less, 6 or less, 5 or less, 4 or less, 3 or less, or 2 or less. A smaller number of substitutions, additions, and deletions is preferable, though a large number may be available as long as the amino acid sequence retains biological activity (preferably, similar to, or substantially the same as, that of a DISC1 gene product).

In another preferred embodiment, the biological activity possessed by the above-described variant polypeptide includes, but is not limited to, interaction with an antibody specific to a polypeptide consisting of the amino acid sequence set forth in SEQ ID NO. 2 or a fragment thereof, interaction with FEZ1, and the like.

In another preferred embodiment, the above-described alleic mutant preferably has at least 99% homology to the nucleic acid sequence set forth in SEQ ID NO. 1.

The above-described species homolog can be identified by searching a gene sequence database of the species, if any, using DISC1 of the present invention as a query sequence for the database. Alternatively, the species homolog can be identified by using the whole or a part of DISC1 of the present invention as a probe or a primer to screen gene libraries of the species. Such identification methods are well known in the art and are described in documents mentioned herein. The species homolog preferably has at least about 30% homology to the nucleic acid sequence set forth in SEQ ID NO. 1, for example.

In a preferred embodiment, the identity to any one of the polynucleotides (a) to (e) or a complementary sequence thereof may be at least about 80%, more preferably at least about 90%, even more preferably at least about 98%, and most preferably at least about 99%.

In a preferred embodiment, the agent of the present invention is selected from the group consisting of a nucleic acid molecule, a polypeptide, a lipid, a sugar chain, a low molecular weight organic molecule and a composite molecule.

In a preferred embodiment, the agent of the present invention may be a nucleic acid molecule. When the agent of the present invention is a nucleic acid molecule, such a nucleic acid molecule may have at least 8 contiguous nucleotides in length. The nucleic acid molecule of the present invention may have an appropriate nucleotide length which varies depending on the purpose of an application of the present invention. More preferably, the nucleic acid molecule of the present invention may have at least 10 contiguous nucleotides in length, more preferably at least 15 contiguous nucleotides in length, and even more preferably at least 20 contiguous nucleotides in length. The lower limit of the nucleotide length may be values (e.g., 9, 11, 12, 13, 14, 16, etc.) between the above-described specific values, or values (e.g., 21, 22, . . . , 30, etc.) more than the above-described specific values. The upper limit of the length of the nucleic acid molecule of the present invention may be the full length of the sequence set forth in SEQ ID NO. 1 or more as long as the nucleic acid molecule can be used in an application of interest (e.g., a marker, a primer, a probe, etc.). Alternatively, when the nucleic acid molecule is used as a primer, it may typically have at least about 8 nucleotides in length, and preferably about 10 nucleotides in length. When the nucleic acid molecule is used as a probe, it may typically have at least about 15 nucleotides in length, and preferably about 17 nucleotides in length.

Therefore, in one illustrative embodiment, the agent of the present invention may be a nucleic acid molecule having a sequence having at least 70% identity to the nucleic acid sequence of any one of the polynucleotides of (a) to (g) or a complementary sequence thereof.

In another illustrative embodiment, the agent of the present invention may be a nucleic acid molecule hybridizable to the nucleic acid sequence of any one of the polynucleotides of (a) to (g) under stringent conditions.

In a preferred embodiment, a polynucleotide or polypeptide with which the agent of the present invention specifically interacts comprises a range encoding nucleotides 1095 to 2615 in SEQ ID NO. 1 or a range of amino acids 348 to 854 in SEQ ID NO. 2. In another preferred embodiment, the preferable range includes a range encoding a range selected from the group consisting of nucleotides 1095 to 1844, nucleotides 1845 to 2615, nucleotides 1095 to 1952, nucleotides 1095 to 1652, nucleotides 1653 to 1952, nucleotides 1391 to 1652, and nucleotides 1391 to 1952 in SEQ ID NO. 1, or a range selected from the group consisting of amino acids 348 to 597, amino acids 598 to 854, amino acids 348 to 633, amino acids 348 to 533, amino acids 534 to 633, amino acids 446 to 533, and amino acids 446 to 633 in SEQ ID NO. 2.

In a preferred embodiment, the agent of the present invention may be advantageously label or labelable. When labeled, various conditions which can be detected with the agent of the present invention can be directly and/or easily measured. Such a label includes any distinguishable label, including, for example, but being limited to, fluorescent labels, chemiluminescent labels, radiation labels, and the like. Alternatively, when interaction of the agent is performed via an immunological reaction (i.e., with an antibody, etc.), a commonly used system, such as a biotin-streptavidin system or the like, may be available.

In a preferred embodiment, the agent of the present invention may be used to measure the level of binding to FEZ1.

In another aspect, the present invention relates to an agent specifically interacting with DISC1 polypeptide. Representatively, DISC1 polypeptide herein comprises:

(a) a polypeptide consisting of an amino acid sequence set forth in SEQ ID NO. 2 or a fragment thereof;

(b) a polypeptide having an amino acid sequence set forth in SEQ ID NO. 2 or a fragment thereof, wherein at least one amino acid in the sequence has a mutation selected from the group consisting of substitution, addition, and deletion and wherein the polypeptide has biological activity;

(c) a polypeptide encoded by a spliced mutant or alleic mutant of a base sequence set forth in SEQ ID NO. 1;

(d) a polypeptide being a species homolog of an amino acid sequence set forth in SEQ ID NO. 2; or

(e) a polypeptide having an amino acid sequence having at least 70% identity to any one of the polypeptides (a) to (d), and having biological activity.

In a preferred embodiment, the number of substitutions, additions, and deletions in (b) may be preferably limited to, for example, 50 or less, 40 or less, 30 or less, 20 or less, 15 or less, 10 or less, 9 or less, 8 or less, 7 or less, 6 or less, 5 or less, 4 or less, 3 or less, or 2 or less. A smaller number of substitutions, additions, and deletions is preferable, though a large number may be available as long as the amino acid sequence retains biological activity (preferably, similar to, or substantially the same as, that of a DISC1 gene product).

In another preferred embodiment, the above-described alleic mutant of (c) preferably has at least about 99% homology to the amino acid sequence set forth in SEQ ID NO. 2.

In another preferred embodiment, the above-described species homolog can be identified as described herein above and preferably has at least about 30% homology to the amino acid sequence set forth in SEQ ID NO. 2.

In another preferred embodiment, the biological activity possessed by the above-described variant polypeptide of (e) includes, but is not limited to, interaction with an antibody specific to a polypeptide consisting of the amino acid sequence set forth in SEQ ID NO. 2 or a fragment thereof, interaction with FEZ1, and the like.

In a preferred embodiment, the identity to any one of the polypeptides of (a) to (d) may be at least about 80%, more preferably at least about 90%, even more preferably at least about 98%, and most preferably at least about 99%. The polypeptide with which the agent of the present invention specifically interacts typically has a sequence of at least 3 contiguous amino acids. The amino acid length of the polypeptide of the present invention may have any short length as long as the polypeptide is suitable for an application of interest. Preferably, a longer sequence may be used. Therefore, the polypeptide of the present invention preferably has at least 4 amino acids in length, more preferably 5 amino acids in length, 6 amino acids in length, 7 amino acids in length, 8 amino acids in length, 9 amino acids in length, or 10 amino acids in length, even more preferably at least 15 amino acids in length, and still even more preferably at least 20 amino acids in length. The lower limit of the amino acid length may be values (e.g., 11, 12, 13, 14, 16, etc.) between the above-described specific values, or values (e.g., 21, 22, . . . , 30, etc.) more than the above-described specific values. The upper limit of the length of the polypeptide of the present invention may be equal to the full length of the sequence set forth in SEQ ID NO. 2 or more as long as the polypeptide can interact with a certain agent.

In a preferred embodiment, the agent of the present invention is selected from the group consisting of a nucleic acid molecule, a polypeptide, a lipid, a sugar chain, a low molecular weight organic molecule, and a composite molecule thereof. More preferably, the agent of the present invention is an antibody or a derivative thereof (e.g., a single chain antibody, etc.). Therefore, the agent of the present invention can be used as a probe.

In a preferred embodiment, the polypeptide with which the agent of the present invention specifically interacts comprises a range of amino acids 348 to 854 in SEQ ID NO. 2.

In another preferred embodiment, the preferable range includes a range selected from the group consisting of amino acids 348 to 597, amino acids 598 to 854, amino acids 348 to 633, amino acids 348 to 533, amino acids 534 to 633, amino acids 446 to 533, and amino acids 446 to 633 in SEQ ID NO. 2.

In a preferred embodiment, the agent of the present invention may be advantageously label or labelable. When labeled, various conditions which can be detected with the agent of the present invention can be directly and/or easily measured. Such a label includes any distinguishable label, including, for example, but being limited to, fluorescent labels, chemiluminescent labels, radiation labels, and the like. Alternatively, when interaction of the agent is performed via an immunological reaction (i.e., with an antibody, etc.), a commonly used system, such as a biotin-streptavidin system or the like, may be available.

In a preferred embodiment, the agent of the present invention may be used to measure the level of binding to FEZ1.

In another aspect, the present invention relates to an agent specifically interacting with a polynucleotide encoding FEZ1 or a fragment thereof. Representatively, FEZ1 comprises:

(a) a polynucleotide having a base sequence set forth in SEQ ID NO. 3 or a fragment thereof;

(b) a polynucleotide encoding a polypeptide consisting of an amino acid sequence set forth in SEQ ID NO. 4 or a fragment thereof;

(c) a polynucleotide encoding a variant polypeptide having an amino acid sequence set forth in SEQ ID NO. 4 or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 3;

(e) a polynucleotide encoding a species homolog of a polypeptide consisting of an amino acid sequence set forth in SEQ ID NO. 4;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity.

In a preferred embodiment; the number of substitutions, additions, and deletions in (c) may be preferably limited to, for example, 50 or less, 40 or less, 30 or less, 20 or less, 15 or less, 10 or less, 9 or less, 8 or less, 7 or less, 6 or less, 5 or less, 4 or less, 3 or less, or 2 or less. A smaller number of substitutions, additions, and deletions is preferable, though a large number may be available as long as the amino acid sequence retains biological activity (preferably, similar to, or substantially the same as, that of a FEZ1 gene product).

In another preferred embodiment, the biological activity possessed by the above-described variant polypeptide includes, but is not limited to, interaction with an antibody specific to a polypeptide consisting of the amino acid sequence set forth in SEQ ID NO. 4 or a fragment thereof, interaction with FEZ1, and the like.

In another preferred embodiment, the above-described alleic mutant preferably has at least 99% homology to the nucleic acid sequence set forth in SEQ ID NO. 3.

The above-described species homolog can be identified by searching a gene sequence database of the species, if any, using FEZ1 of the present invention as a query sequence for the database. Alternatively, the species homolog can be identified by using the whole or a part of FEZ1 of the present invention as a probe or a primer to screen gene libraries of the species. Such identification methods are well known in the art and are described in documents mentioned herein. The species homolog preferably has at least about 30% homology to the nucleic acid sequence set forth in SEQ ID NO. 3, for example.

In a preferred embodiment, the identity to any one of the polynucleotides (a) to (e) or a complementary sequence thereof may be at least about 80%, more preferably at least about 90%, even more preferably at least about 98%, and most preferably at least about 99%.

In a preferred embodiment, the agent of the present invention is selected from the group consisting of a nucleic acid molecule, a polypeptide, a lipid, a sugar chain, a low molecular weight organic molecule and a composite molecule.

In a preferred embodiment, the agent of the present invention may be a nucleic acid molecule. When the agent of the present invention is a nucleic acid molecule, such a nucleic acid molecule may have at least 8 contiguous nucleotides in length. The nucleic acid molecule of the present invention may have an appropriate nucleotide length which varies depending on the purpose of an application of the present invention. More preferably, the nucleic acid molecule of the present invention may have at least 10 contiguous nucleotides in length, more preferably at least 15 contiguous nucleotides in length, and even more preferably at least 20 contiguous nucleotides in length. The lower limit of the nucleotide length may be values (e.g., 9, 11, 12, 13, 14, 16, etc.) between the above-described specific values, or values (e.g., 21, 22, . . . , 30, etc.) more than the above-described specific values. The upper limit of the length of the nucleic acid molecule of the present invention may be the full length of the sequence set forth in SEQ ID NO. 3 or more as long as the nucleic acid molecule can be used in an application of interest (e.g., a marker, a primer, a probe, etc.). Alternatively, when the nucleic acid molecule is used as a primer, it may typically have at least about 8 nucleotides in length, and preferably about 10 nucleotides in length. When the nucleic acid molecule is used as a probe, it may typically have at least about 15 nucleotides in length, and preferably about 17 nucleotides in length.

Therefore, in one illustrative embodiment, the agent of the present invention may be a nucleic acid molecule having a sequence having at least 70% identity to the nucleic acid sequence of any one of the polynucleotides of (a) to (g) or a complementary sequence thereof.

In another illustrative embodiment, the agent of the present invention may be a nucleic acid molecule hybridizable to the nucleic acid sequence of any one of the polynucleotides of (a) to (g) the under stringent conditions.

In a preferred embodiment, the polynucleotide or the polypeptide with which the agent of the present invention specifically interacts comprises a range encoding nucleotides 478 to 1269 in SEQ ID NO. 3 or a range of amino acids 129 to 392 in SEQ ID NO. 4. In another preferred embodiment, the preferable range includes a range encoding nucleotides 832 to 1269 in SEQ ID NO. 3, or a range of amino acids 247 to 392 in SEQ ID NO. 4.

In a preferred embodiment, the agent of the present invention may be advantageously label or labelable. When labeled, various conditions which can be detected with the agent of the present invention can be directly and/or easily measured. Such a label includes any distinguishable label, including, for example, but being limited to, fluorescent labels, chemiluminescent labels, radiation labels, and the like. Alternatively, when interaction of the agent is performed via an immunological reaction (i.e., with an antibody, etc.), a commonly used system, such as a biotin-streptavidin system or the like, may be available.

In a preferred embodiment, the agent of the present invention may be used to measure the level of binding to DISC1.

In another aspect, the present invention relates to an agent specifically interacting with FEZ1 polypeptide. Representatively, FEZ1 polypeptide comprises:

(a) a polypeptide consisting of an amino acid sequence set forth in SEQ ID NO. 4 or a fragment thereof;

(b) a polypeptide having an amino acid sequence set forth in SEQ ID NO. 4 or a fragment thereof, wherein at least one amino acid in the sequence has a mutation selected from the group consisting of substitution, addition, and deletion and wherein the polypeptide has biological activity;

(c) a polypeptide encoded by a spliced mutant or alleic mutant of a base sequence set forth in SEQ ID NO. 3;

(d) a polypeptide being a species homolog of an amino acid sequence set forth in SEQ ID NO. 4; or

(e) a polypeptide having an amino acid sequence having at least 70% identity to any one of the polypeptides (a) to (d), and having biological activity.

In a preferred embodiment, the number of substitutions, additions, and deletions in (b) may be preferably limited to, for example, 50 or less, 40 or less, 30 or less, 20 or less, 15 or less, 10 or less, 9 or less, 8 or less, 7 or less, 6 or less, 5 or less, 4 or less, 3 or less, or 2 or less. A smaller number of substitutions, additions, and deletions is preferable, though a large number may be available as long as the amino acid sequence retains biological activity (preferably, similar to, or substantially the same as, that of a FEZ1 gene product).

In another preferred embodiment, the above-described alleic mutant of (c) preferably has at least 99% homology to the nucleic acid sequence set forth in SEQ ID NO. 4.

In another preferred embodiment, the above-described species homolog can be identified as described above. The species homolog preferably has at least about 30% homology to the nucleic acid sequence set forth in SEQ ID NO. 4.

In another preferred embodiment, the biological activity possessed by the above-described variant polypeptide of (e) includes, but is not limited to, interaction with an antibody specific to a polypeptide consisting of the amino acid sequence set forth in SEQ ID NO. 4 or a fragment thereof, interaction with DISC1, and the like.

In a preferred embodiment, the identity to any one of the polynucleotides (a) to (d) or a complementary sequence thereof may be at least about 80%, more preferably at least about 90%, even more preferably at least about 98%, and most preferably at least about 99%.

The polypeptide with which the agent of the present invention specifically interacts typically has a sequence of at least 3 contiguous amino acids. The amino acid length of the polypeptide of the present invention may have any short length as long as the polypeptide is suitable for an application of interest. Preferably, a longer sequence may be used. Therefore, the polypeptide of the present invention preferably has at least 4 amino acids in length, more preferably 5 amino acids in length, 6 amino acids in length, 7 amino acids in length, 8 amino acids in length, 9 amino acids in length, or 10 amino acids in length, even more preferably at least 15 amino acids in length, and still even more preferably at least 20 amino acids in length. The lower limit of the amino acid length may be values (e.g., 11, 12, 13, 14, 16, etc.) between the above-described specific values, or values (e.g., 21, 22, . . . , 30, etc.) more than the above-described specific values. The upper limit of the length of the polypeptide of the present invention may be equal to the full length of the sequence set forth in SEQ ID NO. 4 or more as long as the polypeptide can interact with a certain agent.

In a preferred embodiment, the agent of the present invention is selected from the group consisting of a nucleic acid molecule, a polypeptide, a lipid, a sugar chain, a low molecular weight organic molecule and a composite molecule. More preferably, the agent of the present invention is an antibody or a derivative thereof (e.g., a single chain antibody, etc.). Therefore, the agent of the present invention can be used as a probe.

In a preferred embodiment, the polypeptide with which the agent of the present invention specifically interacts comprises a range of amino acids 129 to 392 in SEQ ID NO. 4. In another preferred embodiment, the preferable range includes a range of amino acids 247 to 392 in SEQ ID NO. 4.

In a preferred embodiment, the agent of the present invention may be advantageously label or labelable. When labeled, various conditions which can be detected with the agent of the present invention can be directly and/or easily measured. Such a label includes any distinguishable label, including, for example, but being limited to, fluorescent labels, chemiluminescent labels, radiation labels, and the like. Alternatively, when interaction of the agent is performed via an immunological reaction (i.e., with an antibody, etc.), a commonly used system, such as a biotin-streptavidin system or the like, may be available.

In a preferred embodiment, the agent of the present invention may be used to measure the level of binding to DISC1.

In one aspect, the present invention relates to an agent specifically interacting with a polynucleotide encoding KIAA0844 or a fragment thereof. Representatively, KIAA0844 comprises:

(a) a polynucleotide having abase sequence set forth in SEQ ID NO. 13 or a fragment thereof;

(b) a polynucleotide encoding a polypeptide consisting of an amino acid sequence set forth in SEQ ID NO. 14 or a fragment thereof;

(c) a polynucleotide encoding a variant polypeptide having an amino acid sequence set forth in SEQ ID NO. 14 or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 13;

(e) a polynucleotide encoding a species homolog of a polypeptide consisting of an amino acid sequence set forth in SEQ ID NO. 14;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity.

In one preferred embodiment, the number of substitutions, additions, and deletions in (c) is preferably limited, for example, 50 or less, 40 or less, 30 or less, 20 or less, 15 or less, 10 or less, 9 or less, 8 or less, 7 or less, 6 or less, 5 or less, 4 or less, 3 or less, or 2 or less. A smaller number of substitutions, additions, and deletions is preferable, though a large number may be available as long as the amino acid sequence retains biological activity (preferably, similar to, or substantially the same as, that of a KIAA0844 gene product).

In another preferred embodiment, the biological activity possessed by the above-described variant polypeptide includes, but is not limited to, interaction with an antibody specific to a polypeptide consisting of the amino acid sequence set forth in SEQ ID NO. 14 or a fragment thereof, interaction with KIAA0844, and the like.

In another preferred embodiment, the above-described alleic mutant preferably has at least 99% homology to the nucleic acid sequence set forth in SEQ ID NO. 13.

The above-described species homolog can be identified by searching a gene sequence database of the species, if any, using KIAA0844 of the present invention as a query sequence for the database. Alternatively, the species homolog can be identified by using the whole or a part of KIAA0844 of the present invention as a probe or a primer to screen gene libraries of the species. Such identification methods are well known in the art and are described in documents mentioned herein. The species homolog preferably has at least about 30% homology to the nucleic acid sequence set forth in SEQ ID NO. 13, for example.

In a preferred embodiment, the identity to any one of the polynucleotides (a) to (e) or a complementary sequence thereof may be at least about 80%, more preferably at least about 90%, even more preferably at least about 98%, and most preferably at least about 99%.

In a preferred embodiment, the agent of the present invention is selected from the group consisting of a nucleic acid molecule, a polypeptide, a lipid, a sugar chain, a low molecular weight organic molecule and a composite molecule.

In a preferred embodiment, the agent of the present invention may be a nucleic acid molecule. When the agent of the present invention is a nucleic acid molecule, such a nucleic acid molecule may have at least 8 contiguous nucleotides in length. The nucleic acid molecule of the present invention may have an appropriate nucleotide length which varies depending on the purpose of an application of the present invention. More preferably, the nucleic acid molecule of the present invention may have at least 10 contiguous nucleotides in length, more preferably at least 15 contiguous nucleotides in length, and even more preferably at least 20 contiguous nucleotides in length. The lower limit of the nucleotide length may be values (e.g., 9, 11, 12, 13, 14, 16, etc.) between the above-described specific values, or values (e.g., 21, 22, . . . , 30, etc.) more than the above-described specific values. The upper limit of the length of the nucleic acid molecule of the present invention may be the full length of the sequence set forth in SEQ ID NO. 13 or more as long as the nucleic acid molecule can be used in an application of interest (e.g., a marker, a primer, a probe, etc.). Alternatively, when the nucleic acid molecule is used as a primer, it may typically have at least about 8 nucleotides in length, and preferably about 10 nucleotides in length. When the nucleic acid molecule is used as a probe, it may typically have at least about 15 nucleotides in length, and preferably about 17 nucleotides in length.

Therefore, in one illustrative embodiment, the agent of the present invention may be a nucleic acid molecule having a sequence having at least 70% identity to the nucleic acid sequence of any one of the polynucleotides of (a) to (g) or a complementary sequence thereof.

In another illustrative embodiment, the agent of the present invention may be a nucleic acid molecule hybridizable to the nucleic acid sequence of any one of the polynucleotides of (a) to (g) under stringent conditions.

In a preferred embodiment, the agent of the present invention may be advantageously label or labelable. When labeled, various conditions which can be detected with the agent of the present invention can be directly and/or easily measured. Such a label includes any distinguishable label, including, for example, but being limited to, fluorescent labels, chemiluminescent labels, radiation labels, and the like. Alternatively, when interaction of the agent is performed via an immunological reaction (i.e., with an antibody, etc.), a commonly used system, such as a biotin-streptavidin system or the like, may be available.

In a preferred embodiment, the agent of the present invention may be used to measure the level of binding to DISC1 or FEZ1.

In another aspect, the present invention relates to an agent specifically interacting with KIAA0844 polypeptide. Representatively, KIAA0844 polypeptide herein comprises:

(a) a polypeptide consisting of an amino acid sequence set forth in SEQ ID NO. 14 or a fragment thereof;

(b) a polypeptide having an amino acid sequence set forth in SEQ ID NO. 14 or a fragment thereof, wherein at least one amino acid in the sequence has a mutation selected from the group consisting of substitution, addition, and deletion and wherein the polypeptide has biological activity;

(c) a polypeptide encoded by a spliced mutant or alleic mutant of a base sequence set forth in SEQ ID NO. 13;

(d) a polypeptide being a species homolog of an amino acid sequence set forth in SEQ ID NO. 14; or

(e) a polypeptide having an amino acid sequence having at least 70% identity to any one of the polypeptides (a) to (d), and having biological activity.

In a preferred embodiment, the number of substitutions, additions, and deletions in (b) may be preferably limited to, for example, 50 or less, 40 or less, 30 or less, 20 or less, 15 or less, 10 or less, 9 or less, 8 or less, 7 or less, 6 or less, 5 or less, 4 or less, 3 or less, or 2 or less. A smaller number of substitutions, additions, and deletions is preferable, though a large number may be available as long as the amino acid sequence retains biological activity (preferably, similar to, or substantially the same as, that of a KIAA0844 gene product).

In another preferred embodiment, the above-described alleic mutant of (c) preferably has at least 99% homology to the amino acid sequence set forth in SEQ ID NO. 14.

In another preferred embodiment, the biological activity possessed by the above-described variant polypeptide of (e) includes, but is not limited to, interaction with an antibody specific to a polypeptide consisting of the amino acid sequence set forth in SEQ ID NO. 14 or a fragment thereof, interaction with DISC1 or FEZ1, and the like.

In a preferred embodiment, the identity to any one of the polypeptides of (a) to (d) may be at least about 80. %, more preferably at least about 90%, even more preferably at least about 98%, and most preferably at least about 99%.

The polypeptide with which the agent of the present invention specifically interacts typically has a sequence of at least 3 contiguous amino acids. The amino acid length of the polypeptide of the present invention may have any short length as long as the polypeptide is suitable for an application of interest. Preferably, a longer sequence may be used. Therefore, the polypeptide of the present invention preferably has at least 4 amino acids in length, more preferably 5 amino acids in length, 6 amino acids in length, 7 amino acids in length, 8 amino acids in length, 9 amino acids in length, or 10 amino acids in length, even more preferably at least 15 amino acids in length, and still even more preferably at least 20 amino acids in length. The lower limit of the amino acid length may be values (e.g., 11, 12, 13, 14, 16, etc.) between the above-described specific values, or values (e.g., 21, 22, . . . , 30, etc.) more than the above-described specific values. The upper limit of the length of the polypeptide of the present invention may be equal to the full length of the sequence set forth in SEQ ID NO. 14 or more as long as the polypeptide can interact with a certain agent.

In a preferred embodiment, the agent of the present invention is selected from the group consisting of a nucleic acid molecule, a polypeptide, a lipid, a sugar chain, a low molecular weight organic molecule, and a composite molecule thereof. More preferably, the agent of the present invention is an antibody or a derivative thereof (e.g., a single chain antibody, etc.). Therefore, the agent of the present invention can be used as a probe.

In a preferred embodiment, the agent of the present invention may be advantageously label or labelable. When labeled, various conditions which can be detected with the agent of the present invention can be directly and/or easily measured. Such a label includes any distinguishable label, including, for example, but being limited to, fluorescent labels, chemiluminescent labels, radiation labels, and the like. Alternatively, when interaction of the agent is performed via an immunological reaction (i.e., with an antibody, etc.), a commonly used system, such as a biotin-streptavidin system or the like, may be available.

In a preferred embodiment, the agent of the present invention may be used to measure the level of binding to DISC1 or FEZ1.

In another aspect, the present invention provides a composition for determining a function of FEZ1 or KIAA0844. The composition comprises:

(A) an agent specifically interacting with:

(a) a polynucleotide having a base sequence set forth in SEQ ID NO. 1 or a fragment thereof;

(b) a polynucleotide encoding a polypeptide consisting of an amino acid sequence set forth in SEQ ID NO. 2 or a fragment thereof;

(c) a polynucleotide encoding a variant polypeptide having an amino acid sequence set forth in SEQ ID NO. 2 or a fragment thereof, wherein at least one amino acid in the sequence has a mutation selected from the group consisting of substitution, addition, and deletion and wherein the variant polypeptide has biological activity:

(d) a polynucleotide, which is a spliced mutant or alleic mutant of a base sequence set forth in SEQ ID NO. 1;

(e) a polynucleotide encoding a species homolog of a polypeptide consisting of an amino acid sequence set forth in SEQ ID NO. 2;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides

(a) to (e) or a complementary sequence thereof, and encoding a polypeptide having biological activity; and/or

(B) an agent specifically interacting with a polypeptide, wherein the polypeptide comprises:

(a) a polypeptide consisting of an amino acid sequence set forth in SEQ ID NO. 2 or a fragment thereof;

(b) a polypeptide having an amino acid sequence set forth in SEQ ID NO. 2 or a fragment thereof, wherein at least one amino acid in the sequence has a mutation selected from the group consisting of substitution, addition, and deletion and wherein the polypeptide has biological activity;

(c) a polypeptide encoded by a spliced mutant or alleic mutant of a base sequence set forth in SEQ ID NO. 1;

(d) a polypeptide being a species homolog of an amino acid sequence set forth in SEQ ID NO. 2; or

(e) a polypeptide having an amino acid sequence having at least 70% identity to any one of the polypeptides (a) to (d), and having biological activity.

The agent contained in the composition may be in any form described herein.

In another aspect, the present invention provides a composition for determining a function of DISC1 or KIAA0844. The composition comprises:

(A) an agent specifically interacting with:

(a) a polynucleotide having a base sequence set forth in SEQ ID NO. 3 or a fragment thereof;

(b) a polynucleotide encoding a polypeptide consisting of an amino acid sequence set forth in SEQ ID NO. 4 or a fragment thereof;

(c) a polynucleotide encoding a variant polypeptide having an amino acid sequence set forth in SEQ ID NO. 4 or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 3;

(e) a polynucleotide encoding a species homolog of a polypeptide consisting of an amino acid sequence set forth in SEQ ID NO. 4;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity; and/or

(B) an agent specifically interacting with a polypeptide, wherein the polypeptide comprises:

(a) a polypeptide consisting of an amino acid sequence set forth in SEQ ID NO. 4 or a fragment thereof;

(b) a polypeptide having an amino acid sequence set forth in SEQ ID NO. 4 or a fragment thereof, wherein at least one amino acid in the sequence has a mutation selected from the group consisting of substitution, addition, and deletion and wherein the polypeptide has biological activity;

(c) a polypeptide encoded by a spliced mutant or alleic mutant of a base sequence set forth in SEQ ID NO. 3;

(d) a polypeptide being a species homolog of an amino acid sequence set forth in SEQ ID NO. 4; or

(e) a polypeptide having an amino acid sequence having at least 70% identity to any one of the polypeptides (a) to (d), and having biological activity.

The agent contained in the composition may be in any form described herein.

In another aspect, the present invention provides a composition for determining a function of FEZ1 or DISC1. The composition comprises:

(A) an agent specifically interacting with:

(a) a polynucleotide having a base sequence set forth in SEQ ID NO. 13 or a fragment thereof;

(b) a polynucleotide encoding a polypeptide consisting of an amino acid sequence set forth in SEQ ID NO. 14 or a fragment thereof;

(c) a polynucleotide encoding a variant polypeptide having an amino acid sequence set forth in SEQ ID NO. 14 or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 13;

(e) a polynucleotide encoding a species homolog of a polypeptide consisting of an amino acid sequence set forth in SEQ ID NO. 14;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides

(a) to (e) or a complementary sequence thereof, and encoding a polypeptide having biological activity: and/or

(B) an agent specifically interacting with a polypeptide, wherein the polypeptide comprises:

(a) a polypeptide consisting of an amino acid sequence set forth in SEQ ID NO. 14 or a fragment thereof;

(b) a polypeptide having an amino acid sequence set forth in SEQ ID NO. 14 or a fragment thereof, wherein at least one amino acid in the sequence has a mutation selected from the group consisting of substitution, addition, and deletion and wherein the polypeptide has biological activity:

(c) a polypeptide encoded by a spliced mutant or alleic mutant of a base sequence set forth in SEQ ID NO. 13;

(d) a polypeptide being a species homolog of an amino acid sequence set forth in SEQ ID NO. 14; or

(e) a polypeptide having an amino acid sequence having at least 70% identity to any one of the polypeptides (a) to (d), and having biological activity.

The agent contained in the composition may be in any form described herein.

In another aspect, the present invention provides a composition for determining a level of axon outgrowth and/or fasciculation, or a condition, disorder or disease associated with the level. The composition comprises:

(A) an agent specifically interacting with:

(a) a polynucleotide having a base sequence set forth in SEQ ID NO. 1 or a fragment thereof;

(b) a polynucleotide encoding a polypeptide consisting of an amino acid sequence set forth in SEQ ID NO. 2 or a fragment thereof;

(c) a polynucleotide encoding a variant polypeptide having an amino acid sequence set forth in SEQ ID NO. 2 or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 1;

(e) a polynucleotide encoding a species homolog of a polypeptide consisting of an amino acid sequence set forth in SEQ ID NO. 2;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity;

(B) an agent specifically interacting with a polypeptide, wherein the polypeptide comprises:

(a) a polypeptide consisting of an amino acid sequence set forth in SEQ ID NO. 2 or a fragment thereof;

(b) a polypeptide having an amino acid sequence set forth in SEQ ID NO. 2 or a fragment thereof, wherein at least one amino acid in the sequence has a mutation selected from the group consisting of substitution, addition, and deletion and wherein the polypeptide has biological activity;

(c) a polypeptide encoded by a spliced mutant or alleic mutant of a base sequence set forth in SEQ ID NO. 1;

(d) a polypeptide being a species homolog of an amino acid sequence set forth in SEQ ID NO. 2; or

(e) a polypeptide having an amino acid sequence having at least 70-% identity to any one of the polypeptides (a) to (d), and having biological activity;

(C) an agent specifically interacting with:

(a) a polynucleotide having abase sequence set forth in SEQ ID NO. 3 or a fragment thereof;

(b) a polynucleotide encoding a polypeptide consisting of an amino acid sequence set forth in SEQ ID NO. 4 or a fragment thereof;

(c) a polynucleotide encoding a variant polypeptide having an amino acid sequence set forth in SEQ ID NO. 4 or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 3;

(e) a polynucleotide encoding a species homolog of a polypeptide consisting of an amino acid sequence set forth in SEQ ID NO. 4;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity; and/or

(D) an agent specifically interacting with a polypeptide, wherein the polypeptide comprises:

(a) a polypeptide consisting of an amino acid sequence set forth in SEQ ID NO. 4 or a fragment thereof;

(b) a polypeptide having an amino acid sequence set forth in SEQ ID NO. 4 or a fragment thereof, wherein at least one amino acid in the sequence has a mutation selected from the group consisting of substitution, addition, and deletion and wherein the polypeptide has biological activity;

(c) a polypeptide encoded by a spliced mutant or alleic mutant of a base sequence set forth in SEQ ID NO. 3;

(d) a polypeptide being a species homolog of an amino acid sequence set forth in SEQ ID NO. 4; or

(e) a polypeptide having an amino acid sequence having at least 70% identity to any one of the polypeptides (a) to (d), and having biological activity.

The agent contained in the composition may be in any form described herein.

In another aspect, the present invention provides a composition for determining a level of axon outgrowth and/or fasciculation, or a condition, disorder or disease associated with the level. The composition comprises:

(A) an agent specifically interacting with:

(a) a polynucleotide having abase sequence set forth in SEQ ID NO. 13 or a fragment thereof;

(b) a polynucleotide encoding a polypeptide consisting of an amino acid sequence set forth in SEQ ID NO. 14 or a fragment thereof;

(c) a polynucleotide encoding a variant polypeptide having an amino acid sequence set forth in SEQ ID NO. 14 or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 13;

(e) a polynucleotide encoding a species homolog of a polypeptide consisting of an amino acid sequence set forth in SEQ ID NO. 14;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity; and/or

(B) an agent specifically interacting with a polypeptide, wherein the polypeptide comprises:

(a) a polypeptide consisting of an amino acid sequence set forth in SEQ ID NO. 14 or a fragment thereof;

(b) a polypeptide having an amino acid sequence set forth in SEQ ID NO. 14 or a fragment thereof, wherein at least one amino acid in the sequence has a mutation selected from the group consisting of substitution, addition, and deletion and wherein the polypeptide has biological activity;

(c) a polypeptide encoded by a spliced mutant or alleic mutant of a base sequence set forth in SEQ ID NO. 13;

(d) a polypeptide being a species homolog of an amino acid sequence set forth in SEQ ID NO. 14; or

(e) a polypeptide having an amino acid sequence having at least 70% identity to any one of the polypeptides (a) to (d), and having biological activity.

The agent contained in the composition may be in any form described herein.

In another aspect, the present invention provides a method for diagnosis of a condition, disorder or disease associated with a level of axon outgrowth and/or fasciculation. The method comprises the steps of:

(a) measuring binding of DISC1 or FEZ1 and KIAA0844 in a sample derived from a subject; and

(b) comparing a level of the measured binding with a level of normal binding, wherein when the level of the measured binding is lower than the level of the normal binding, the level of axon outgrowth and/or fasciculation is inferior to a normal level thereof.

In another preferred embodiment, the condition, disorder or disease associated with the level of axon outgrowth and/or fasciculation is selected from the group consisting of schizophrenia, mental retardation, depression, and epilepsy.

In another aspect, the present invention provides a method for diagnosis of a condition, disorder or disease associated with a level of axon outgrowth and/or fasciculation. The method comprises the steps of:

(a) measuring binding of all or any two of DISC1, FEZ1 and KIAA0844 in a sample derived from a subject; and

(b) comparing a level of the measured binding with a level of normal binding, wherein when the level of the measured binding is lower than the level of the normal binding, the level of axon outgrowth and/or fasciculation is inferior to a normal level thereof.

DISC1, FEZ1 and KIAA0844 may be any one as described herein in detail, and preferably naturally-occurring ones

(i.e., ones present in organisms). The above-described sample may be any one which contains a complex of all or any two or more of DISC1, FEZ1, and KIAA0844 at a measurable level, including, but being limited to, for example, blood, urine, lymph fluid, cerebrospinal fluid, neurons, nervous tissue, biopsy samples, and the like. Preferably, a sample containing neurons may be used. Known techniques can be used to prepare samples. Examples of such preparation methods include, but are not limited to, use of a sample extracted or excised from a subject without modification or suspension of a sample in buffered solution or culture medium. The comparison of binding can be performed using a technique well known in the art. Such a technique includes, but is not limited to, for example, demonstration of co-localization by immunoprecipitation, pull-down assay, or immunological staining. In the method of the present invention, when the level of measured binding is lower than the level of normal binding, it is indicated that a level of axon outgrowth and/or fasciculation is inferior to a normal level thereof. This fact was not known prior to the present invention, the present invention provides an accurate and/or simple method for measuring a level of axon outgrowth and/or fasciculation. The normal level used herein is of a subject who does not exhibit an abnormal level of axon outgrowth and/or fasciculation.

In another aspect, the present invention provides a method for diagnosis of a condition, disorder or disease associated with a level of axon outgrowth and/or fasciculation. The method comprises the steps of:

(a) measuring binding of all or any two of DISC1, FEZ1 and KIAA0844 in a subject in need of diagnosis of a condition, disorder or disease associated with a level of axon outgrowth and/or fasciculation;

(b) measuring binding of all or any two of DISC1, FEZ1 and KIAA0844 in a normal subject; and

(c) comparing a level of the binding of (a) with a level of the binding of (b),

wherein when the level of the binding of (a) is higher or lower than the level of the binding of (b), the subject is diagnosed as having an abnormality, disorder or disease associated with a level of axon outgrowth and/or fasciculation.

When the binding of all or any two of DISC1, FEZ1 and KIAA0844 is measured for a subject in need of diagnosis of a condition, disorder or disease associated with a level of axon outgrowth and/or fasciculation and In a normal subject, a sample may be extracted from the subject or the body of the subject may be directly measured. When the body is directly measured, for example, an agent specifically interacting with all or any of DISC1, FEZ1 and KIAA0844 may be measured by determining whether or not the agent interacts with the mutual binding sites of all or any two of DISC1, FEZ1 and KIAA0844 (particularly, the site of a range selected from the group consisting of amino acids 348 to 854, amino acids 348 to 597, amino acids 598 to 854, amino acids 348 to 633, amino acids 348 to 533, amino acids 534 to 633, amino acids 446 to 533, and amino acids 446 to 633 in SEQ ID NO. 2, and the site of a range selected from the group consisting of amino acids 129 to 392 and amino acids 247 to 392 in SEQ ID NO. 4). In such a case, an agent specifically interacting with portions other than the mutual binding site of all or any two of DISC1, FEZ1 and KIAA0844 can be used as a negative control. In such a case, an agent used may be preferably linked to a matter which can be externally measured (e.g., a radiation label, a label reacting with magnetic resonance, etc.).

In a preferred embodiment, a diagnosis method of the present invention can be used to diagnose schizophrenia, mental retardation, depression, and epilepsy.

Preferably, an agent used in the diagnosis method of the present invention may be an antibody against a first polypeptide having an amino acid sequence having at least 70% homology to SEQ ID NO. 2 or a fragment thereof.

In another preferred embodiment, an agent used in the diagnosis method of the present invention may be an antibody against a second polypeptide having an amino acid sequence having at least 70% homology to SEQ ID NO. 4 or a fragment thereof. Both of the above-described antibodies may be used.

In another aspect, the present invention relates to a method for detecting a genetic mutation associated with a condition, disorder or disease associated with a level of axon outgrowth and/or fasciculation. The method comprises the step of:

detecting in a mutation in a polynucleotide sequence of a DISC1 gene and/or a FEZ1 gene and/or a KIAA0844 gene in a sample.

Preferably, the mutation is linked to a condition, disorder or disease associated with a level of axon outgrowth and/or fasciculation. Techniques for detecting mutations are well known in the art and any technique may be used.

For example, a mutation detecting method using a DNA chip may be used. The present invention is not limited to this. DNA arrays are widely reviewed in “DNA Maikuroarei to Saishin PCR ho [DNA Microarray and Latest PCR Method], Saibo Kogaku Bessatsu [Special issue of Cell Engineering], ShuJunsha.

In another aspect, the present invention provides a kit for diagnosis of a condition, disorder or disease associated with a level of axon outgrowth and/or fasciculation. The kit comprises:

(a) a composition of the present invention; and

(b) instructions,

the instructions describes:
(i) using the composition to measure binding of all or any two of DISC1, FEZ1 and KIAA0844 in a subject in need of diagnosis of the condition, disorder or disease associated with the level of axon outgrowth and/or fasciculation;
(ii) using the composition to measure binding of all or any two of DISC1, FEZ1 and KIAA0844 in a normal subject; and
(iii) comparing a level of the binding of (i) with a level of the binding of (ii),
wherein when the level of the binding of (i) is higher or lower than the level of the binding of (ii), the subject is diagnosed as having an abnormality, disorder or disease associated with a level of axon outgrowth and/or fasciculation.

The above-described DISC1, FEZ1, KIAA0844 and the like may be in any form described herein. Techniques for measuring the above-described binding of all or any two of DISC1, FEZ1 and KIAA0844 are well known in the art. Various methods may be used as described in somewhere else herein. The instructions for the kit of the present invention may be provided in any form with which the instructions can be conveyed, including paper, computer-readable recording media (e.g., flexibledisks, CD-R), electronicmail, website, etc.).

In a preferred embodiment, the present invention provides a kit for detecting a condition, disorder or disease associated with a level of axon outgrowth and/or fasciculation in a subject. The kit comprises:

(a) at least 2 primers of at least 10 nucleotides in length, wherein each of the primers comprises:
(i) a polynucleotide having at least 70% homology to a polynucleotide set forth in SEQ ID NO. 1; and
(ii) a primer having at least 70% homology to a polynucleotide encoding an amino acid sequence set forth in SEQ ID NO. 2 and hybridizable to the polynucleotide at different positions in the amino acid sequence under stringent conditions; and

(b) instructions describing using the primer of (a) to detect a nucleic acid sequence in a sample derived from the subject and detect a mutation in the polynucleotide set forth in SEQ ID NO. 1.

In a preferred embodiment, the nucleic acid sequence to be detected may be nucleotides 54 to 2615 in SEQ ID NO. 1.

In another preferred embodiment, the present invention provides a kit for detection of a condition, disorder or disease associated with a level of axon outgrowth and/or fasciculation, comprising:

(a) at least 2 primers of at least 10 nucleotides in length, wherein each of the primers comprises:
(i) a polynucleotide having at least 70% homology to a polynucleotide set forth in SEQ ID NO. 13; and
(ii) a primer having at least 70% homology to a polynucleotide encoding an amino acid sequence set forth in SEQ ID NO. 14 and hybridizable to the polynucleotide at different positions in the amino acid sequence under stringent conditions; and

(b) instructions describing using the primer of (a) to detect a nucleic acid sequence in a sample derived from the subject and detect a mutation in the polynucleotide set forth in SEQ ID NO. 13.

In another preferred embodiment, the present invention provides a kit for detecting a condition, disorder or disease associated with a level of axon outgrowth and/or fasciculation in a subject. The kit comprises:

(a) at least 2 primers of at least 10 nucleotides in length, wherein each of the primers comprises:
(i) a polynucleotide having at least 70% homology to a polynucleotide set forth in SEQ ID NO. 3; and
(ii) a primer having at least 70% homology to a polynucleotide encoding an amino acid sequence set forth in SEQ ID NO. 4 and hybridizable to the polynucleotide at different positions in the amino acid sequence under stringent conditions; and

(b) instructions describing using the primer of (a) to detect a nucleic acid sequence in a sample derived from the subject and detect a mutation in the polynucleotide set forth in SEQ ID NO. 3.

In a preferred embodiment, the nucleic acid sequence to be detected may be nucleotides 94 to 1269 in SEQ ID NO. 3.

In the above-described two kits of the present invention, mutations can be detected by performing amplification reactions using a primer and sequencing the amplified products. Sequencing can be performed using a technique utilizing gel or capillary electrophoresis (e.g., using a sequencer commercially available from Applied Biosystems). Alternatively, mutations can be detected using a DNA chip. The present invention is not limited to this.

In another aspect, the present invention provides a method for identifying an agent regulating a condition, disorder or disease associated with a level of axon outgrowth and/or fasciculation. The method comprises:

(a) contacting a first polypeptide having an amino acid sequence having at least 70% homology to SEQ ID NO. 2 or a fragment thereof with a second polypeptide having an amino acid sequence having at least 70% homology to SEQ ID NO. 4 or a fragment thereof in the presence of a test agent; and

(b) comparing a level of binding of the first polypeptide and the second polypeptide with a level of binding therebetween in the absence of the test agent,

wherein when the level of binding in the presence of the test agent is lower than the level of binding in the absence of the test agent, the test agent is a negative-regulatory agent for the condition, disorder or disease associated with the level of axon outgrowth and/or fasciculation; and when the level of binding in the presence of the test agent is higher than the level of binding in the absence of the test agent, the test agent is a positive-regulatory agent for the condition, disorder or disease associated with the level of axon outgrowth and/or fasciculation.

Preferably, the present invention provides a method for diagnosis of a condition, disorder or disease associated with a level of axon outgrowth and/or fasciculation, comprising the steps of:

(a) measuring binding of DISC1 or FEZ1 and KIAA0844 in a subject in need of diagnosis of a condition, disorder or disease associated with a level of axon outgrowth and/or fasciculation;

(b) measuring binding of DISC1 or FEZ1 and KIAA0844 in a normal subject; and (c) comparing a level of the binding of (a) with a level of the binding of (b),

wherein when the level of the binding of (a) is higher or lower than the level of the binding of (b), the subject is diagnosed as having an abnormality, disorder or disease associated with a level of axon outgrowth and/or fasciculation.

Such a screening method is well known in the art, and can be performed using, for example, a microtiter plate, a biological molecule array or chip of DNA or protein. An agent to be tested by screening may be contained in, for example, gene libraries, compound libraries synthesized by combinatorial libraries, and the like. The present invention is not so limited.

Therefore, in a preferred embodiment, the present invention provides a method for identifying a regulatory agent for diseases, such as schizophrenia, mental retardation, depression, and epilepsy. Such a regulatory agent can be used as a pharmaceutical agent for each disease or a lead compound therefor. It is intended to encompass such a regulatory agent, a pharmaceutical agent comprising the regulatory agent, and a therapeutic method using the same within the scope of the present invention.

Therefore, the present invention is intended to provide a drug by computer modeling based on the disclosures of the present invention.

In other embodiments, the present invention includes compounds obtained by a quantitative structure activity relationship (QSAR) computer modeling technique as an instrument for screening for the regulatory activity of the compound of the present invention. Here, the computer technique includes some substrate templates prepared by a computer, pharmacophore, production of homologous models of the active site of the present invention, and the like.

In general, a method for modeling an ordinary characteristic group of a substance capable of interacting with a given substance from data obtained In vitro can be carried out using a CATALYST™ pharmacophore method (Ekins et al., Pharmacogenetics, 9:477-489, 1999; Ekins et al., J. Pharmacol. & Exp. Ther., 288:21-29, 1999; Ekins et al., J. Pharmacol. & Exp. Ther., 290:429-438, 1999; Ekins et al., J. Pharmacol. & Exp. Ther., 291:424-433, 1999) and comparative molecular field analysis; CoMPA) (Jones et al., Drug Metabolism & Disposition, 24:1-6, 1996), and the like. In the present invention, the computer modeling may be carried out using molecular modeling software (e.g., CATALYSTS version 4 (Molecular Simulations, Inc., San Diego, Calif.), etc.).

Fitting of a compound to an active site can be carried out using any computer modeling technique known in the art. Visual inspection and manual operation of a compound to an active site can be carried out using a program, such as QUANTA (Molecular Simulations, Burlington, Mass., 1992), SYBYL (Molecular Modeling Software, Tripos Associates, Inc., St. Louis, Mo., 1992), AMBER (Weiner et al., J. Am. Chem. Soc., 106:765-784, 1984), CHARMM (Brooks et al., J. Comp. Chem., 4:187-217, 1983), or the like. In addition, energy minimization can be carried out using a standard force field, such as CHARMM, AMBER, or the like. Other more specialized computer modelings include GRID (Goodford et al., J. Med. Chem., 28:849-857, 1985), MCSS (Miranker and Karplus, Function and Genetics, 11:29-34, 1991), AUTODOCK (Goodsell and Olsen, Proteins: Structure, Function and Genetics, 8:195-202, 1990), DOCK (Kuntz et al., J. Mol. Biol., 161:269-288, (1982)), and the like. Additional structures of compounds can be newly constructed to blank active sites, active sites of known low molecular weight compounds, or the like, using a computer program, such as LUDI (Bohm, J. Comp. Aid. Molec. Design, 6:61-78, 1992), LEGEND (Nishibata and Itai, Tetrahedron, 47:8985, 1991), LeapFrog (Tripos Associates, St. Louis, Mo.), or the like. Such computer modelings are well known in the art and commonly used. Those skilled in the art can appropriately design compounds within the scope of the present invention in accordance with the disclosures of the present specification.

In a preferred embodiment, the first polypeptide comprises amino acids 446 to 597 in SEQ ID NO. 2.

In another preferred embodiment, the second polypeptide comprises amino acids 247 to 392 in SEQ ID NO. 4.

In a preferred embodiment, the first polypeptide comprises amino acids 446 to 597 in SEQ ID NO. 2 and the second polypeptide comprises amino acids 247 to 392 in SEQ ID NO. 4.

In a preferred embodiment, the step of contacting the above-described polypeptides with each other in the present invention comprises contacting cells expressing the polypeptides of the present invention with each other. Such a contacting method may be performed with any techniques, including, for example, mixing a preparation containing a cell expressing the polypeptide with a preparation to be contacted therewith. The present invention is not limited to this.

In another aspect, the present invention also provides a method for treatment or prophylaxis of a condition, disorder or disease associated with a level of axon outgrowth and/or fasciculation. The method comprises the step of administering a pharmaceutical composition containing a regulatory agent identified by a method of the present invention into a subject. Therefore, in a preferred embodiment, the present invention provides a method for treatment or prophylaxis of schizophrenia, mental retardation, depression, and epilepsy.

In another aspect, the present invention provides a kit for diagnosis of a condition, disorder or disease associated with a level of axon outgrowth and/or fasciculation, comprising:

(a) a composition of the present invention relating to KIAA0844; and

(b) instructions,

the instructions describes:
(i) using the composition to measure binding of DISC1 and FEZ1 in a subject in need of diagnosis of the condition, disorder or disease associated with the level of axon outgrowth and/or fasciculation;
(ii) using the composition to measure binding of DISC1 and FEZ1 in a normal subject; and
(iii) comparing a level of the binding of (i) with a level of the binding of (ii),

wherein when the level of the binding of (i) is higher or lower than the level of the binding of (ii), the subject is diagnosed as having an abnormality, disorder or disease associated with a level of axon outgrowth and/or fasciculation.

In another aspect, the present invention provides a kit for detection of a condition, disorder or disease associated with a level of axon outgrowth and/or fasciculation, comprising:

(a) at least 2 primers of at least 10 nucleotides in length, wherein each of the primers comprises:
(i) a polynucleotide having at least 70% homology to a polynucleotide set forth in SEQ ID NO. 13; and
(ii) a primer having at least 70% homology to a polynucleotide encoding an amino acid sequence set forth in SEQ ID NO. 14 and hybridizable to the polynucleotide at different positions in the amino acid sequence under stringent conditions; and

(b) instructions describing using the primer of (a) to detect a nucleic acid sequence in a sample derived from the subject and detect a mutation in the polynucleotide set forth in SEQ ID NO. 13.

Psychiatric diseases, such as schizophrenia, mental retardation, and the like, have been believed to be difficult to radically treat. However, according to the above-described effects of the present invention, schizophrenia, mental retardation, and the like can be radically treated. Such a radical therapy was conventional considered to be impossible. Therefore, the present invention has usefulness which cannot be achieved by conventional diagnostics and pharmaceutical agents.

Hereinafter, the present invention will be described by way of examples. The examples below are provided only for illustrative purposes. Accordingly, the scope of the present invention is not limited only by the examples except as by the appended claims.

### EXAMPLES

### Example 1

In the examples below, animals were cared for in accordance with rules defined by Osaka University (Japan).

(Material and Methods)

(In Situ Hybridization)

A mouse homologue of DISC1 with a conserved surrounding genomic structure was identified on chromosome 8 and a partial cDNA (SEQ ID NO. 5) of a mouse homologue and a partial cDNA (SEQ ID NO. 9) of a rat homologue were revealed. A fragment of rat cDNA of nucleotide positions 1105-1564 that corresponds to nucleotide positions 1176-1644 of human DISC1 cDNA (SEQ ID NO. 1) was obtained by PCR using the primers:

### 5′-ACACTGAGACAGAGATTGGAAGACCTGG-3′ (SEQ ID NO. 11); and 5′-TGGCAGAGGTCAGAGTCTCTCCCAAGGC-3′ (SEQ ID NO. 12).

Next, the fragment was subcloned into pGEM-T (Promega, Madison, Wis.). Digoxigenin-labeled cRNA probes (antisense and sense) were generated by in vitro transcription using the cDNA fragment as a template in the presence of digoxigenin-labeled dUTP (Roche, Sydney, Australia). Hybridization and posthybridization procedures were performed as described in Katayama et al. (Brain. Res. Mol. Brain. Res., 1998, 56:66-75).

(Yeast Two-Hybrid Screening)

The DISC1 C-terminal domain (amino acids 348-854) was cloned into pAS2-1 (GAL4 DNA-binding domain vector, Clontech, Palo Alto, Calif.) as bait. Yeast strain AH109 was transformed with the bait plasmid, mated with strain Y187 pretransformed with a human adult brain cDNA library (Clontech) and plated on quadruple dropout medium (-Ade, -His, -Leu, -Trp). The screening procedure accompanied with the α-galactosidase assay was performed as described (Clontech Pretransformed MATCHMAKER Libraries User Manual).

To determine the regions involved in the interaction, AH109 was cotransformed with truncated forms of DISC1 and FEZ1 subcloned into pAS2-1 or pACT2 (GAL4 activation domain vector, Clontech), respectively, and then assayed.

(Plasmids)

Full-length DISC1 cDNA and its splicing variant form were cloned into pcDNA3.1 (+) (Invitrogen, Carlsbad, Calif.) and used in Western blot analysis for the detection of endogenous DISC1. DISC1 cDNAs coding a full-length protein, a FEZ1-binding region (amino acids 446-633) and a deleted protein that lacks the binding region were tagged with a FLAG sequence at its 3′ end. Human FEZ1 cDNA was tagged with a HA sequence at its 3′ end. These tagged constructs were cloned into pcDNA3.1 (+) and used in the immunoprecipitation assay. DISC1 cDNA was also cloned into pEGFP-N1 (Clontech) and used in the immunocytochemical analysis. DISC1 cDNA coding a FEZ1-binding region was also cloned into a bicistronic expression vector, pIRES2-EGFP (Clontech), and transfected into stable PC12 cells.

(Cell Culture and Transfection)

HEK293T cells, SK-N-SH cells and PC12 cells were cultured in DMEM containing 10% fetal calf serum (FCS), αMEM/10% FCS and DMEM/10% horse serum/5% FCS, respectively. Hippocampal neurons were prepared from embryonic 18-day Wistar rats as described (Neumann H. et al., Science, 1995, 28:549-552) and cultured in DMEM/10% FCS for 24 hours. The medium was then replaced with DMEM/B27 supplement (Invitrogen). For generation of PC12 cells stably expressing DISC1, FLAG-tagged DISC1 cDNA in pcDNA3.1 (+) was linearized by ScaI and transfected into PC12 cells. Forty-eight hours after transfection, Geneticin (Invitrogen) was added at a concentration of 400 μg/ml. Several clones were picked, expanded in the selective medium and then checked for expression. Mock-stable lines were also generated by the same procedures. For neuronal differentiation, PC12 cells were starved of serum for 4 hours and then treated with NGF at a concentration of 50 ng/ml. For transfection of the cells mentioned above, Lipofectamine 2000 (Invitrogen) was used according to the manufacturer's instructions.

(Antibodies)

Rabbit anti-DISC1 and anti-FEZ1 polyclonal antibodies were raised against SCMTAGVHEAQA of human DISC1 and KVPTLLTDYILKVL of human and rat FEZ1, respectively, and affinity-purified. Monoclonal anti-FLAG (Sigma-Aldrich, St. Louis, Mo.), polyclonal anti-HA (Santa Cruz Biotechnology, Santa Cruz, Calif.) and monoclonal anti-actin (Chemicon, Temecula, Calif.) antibodies were used in immunoprecipitation assays.

(Western Blot)

Cells were homogenized in TNE buffer (20 mM Tris-HCl pH 7.5, 150 mM NaCl, 1 mM EDTA) containing 1% NP40 in the presence of protease inhibitors, incubated on ice for 1 hour and centrifuged at 15000×g for 20 min. Lysates were boiled with SDS sample buffer for 5 min, subjected to SDS-polyacrylamide gel electrophoresis (PAGE) and transferred to PVDF membrane. After blocking with 5% membrane blocking agent (Amersham Biosciences, Buckinghamshire, UK), the membrane was incubated with the primary antibody for 12 hours at 4° C. For the detection of DISC1 and FEZ1, antibodies raised against these proteins were used at 1:500 and 1:250 dilutions, respectively. The membrane was then incubated with anti-rabbit or mouse IgG HRP-linked antibody (Cell Signaling Technology, Beverly, Mass.) at 1:10,000 dilution for 1 hour at room temperature. Immunoblotting was visualized by chemiluminescence using the ECL kit (Amersham Biosciences).

(Immunocytochemistry)

Cells were fixed with 4% paraformaldehyde and permeabilized with 0.3% Triton-X100. After blocking with 3% bovine serum albumin, purified DISC1 and FEZ3 antibodies were applied at 1:100 and 1:50 dilutions, respectively, for 24 hours at 4° C. The secondary antibody (Alexa Flour 594-labeled goat anti-rabbit IgG, Molecular Probes, Eugene, Oreg.) was then applied at a 1:500 dilution for 1 hour at room temperature. For the detection of F-actin, FITC-labeled phalloidin (Sigma-Aldrich) was used at a 1:1000 dilution. Confocal microscopy was performed using a LSM-510 laser scanning microscope (Carl Zeiss, Germany).

(Immunoprecipitation)

HEK293T cells were transfected with DISC1-FLAG and FEZ1-HA, individually or in combination. FLAG-tagged truncated forms of DISC1 mentioned in the Plasmids section were also transfected in combination with FEZ-HA. Cells were lysed in TNE buffer/1% NP40. Prepared lysates were incubated with anti-FLAG antibody for 2 hours at 4° C. and then with Protein Gagarose (Invitrogen) for 1 hour at 4° C. The agarose beads were then washed five times with TNE buffer. Immunoprecipitates were subjected to SDS-PAGE and blotted with anti-HA antibody. Conversely, immunoprecipitates with anti-HA antibody were blotted with anti-FLAG antibody. PC12 cells were transfected with FEZ1-HA or mock and then lysed in TNE buffer/1% NP40. Lysates were immunoprecipitated by anti-HA antibody. Immunoprecipitates were subjected to SDS-PAGE and blotted with anti-actin antibody. Stably DISC1-FLAG-expressing PC12 cells and mock-stable cells were stimulated with NGF (50 ng/ml) for 24 hours and then lysed in TNE buffer/1% NP40. Lysates were immunoprecipitated with anti-FLAG antibody. Immunoprecipitates were subjected to SDS-PAGE and blotted with anti-FEZ1 antibody. Cell lysis and blotting procedures were performed as described in the Western blot section.

(Production of Monoclonal DISC1 Antibody and Monoclonal FEZ1 Antibody)

Monoclonal DISC1 antibody and monoclonal FEZ1 antibody were produced with standard techniques well known in the art (e.g., Kohler and Milstein, Nature (1975) 256:495) or as modified in (e.g., Buck et al., In Vitro, 1982, 18:377). To confirm the production of monoclonal antibodies specifically recognizing DISC1 and FEZ1, a Western blot technique method was used to find that each of the antibodies recognizes a single band.

### Example 2

### Expression of DISC1 in Rat Brain)

DISC1 is reported to be expressed throughout the body (Millar J. K., Wilson-Annan J. C., Anderson S., Christie S., Taylor M. S., Semple C. A. M. et al., Hum. Mol. Genet., 2000, 9: 1415-1423). The present inventors first investigated the distribution of DISC1 mRNA in rat brain by in situ hybridization analysis (FIG. 1). DISC1 was preferentially expressed in hippocampal, cortical, cerebellar and olfactory neurons in adult brain ((a) of FIG. 1). Since a relatively high level of expression was observed in hippocampus, we investigated the DISC1 expression pattern in the hippocampal region at a developing stage. Signals in pyramidal cells of CA1-3 and granule cells of dentate gyrus were more potent at postnatal 7 day than in adulthood ((b) of FIG. 1). This is an intriguing finding in the context of the pathogenesis of schizophrenia where onset usually occurs in young adulthood.

### Example 3

### DISC1 Interacts with FEZ1)

A putative protein of 854 amino acids encoded by the open reading frame in DISC1 ((a) of FIG. 2) has no significant homology to other known proteins (Millar J. K., Wilson-Annan J. C., Anderson S., Christie S., Taylor M. S., Semple C. A. M. et al., Hum. Mol. Genet., 2000, 9:1415-1423). The N-terminal region (amino acids 1-347) of DISC1 is predicted to consist of one or more globular domains (Millar J. K., Wilson-Annan J. C., Anderson S., Christie S., Taylor M. S., Semple C. A. M. et al., Hum. Mol. Genet., 2000, 9:1415-1423). The helical C-terminal region (amino acids 348-854) is predicted to contain the translocation breakpoint and three stretches with coiled-coil forming potential by interaction with other proteins (Millar J. K., Wilson-Annan J. C., Anderson S., Christie S., Taylor M. S., Semple C. A. M. et al., Hum. Mol. Genet., 2000, 9:1415-1423). To confirm the existence of DISC1 protein, we raised an antibody against the C-terminal 12 amino acids of the predicted sequence. This antibody detected two major bands (relative molecular mass Mr; expected size, about 105 K and about 78 K) in lysates from SK-N-SH and HEK293T cells ((b) of FIG. 2). Compared to the sizes of overexpressed DISC1 and its splicing variant that lacks 22 amino acids (Millar J. K., Wilson-Annan J. C., Anderson S., Christie S., Taylor M. S., Semple C. A. M. et al., Hum. Mol. Genet., 2000, 9:1415-1423), endogenous DISC1 appeared to exist as a full-length form and its derivative.

To identify interacting partners of DISC1 that might reflect some biological roles for this protein, we performed a yeast two-hybrid study. A human adult brain cDNA library was screened using the C-terminal region (amino acids 348-854) of DISC1 as bait. One of the positive clones encoded a partial sequence of FEZ1 (amino acids 129-392), which is a mammalian homologue of the Caenorhabditis elegans UNC-76 protein involved in axonal outgrowth and fasciculation (Hall A., Science, 1998, 279: 509-514). UNC-76 and FEZ1 are not similar to any previously characterized proteins and represent a new protein family (Hall A., Science, 1998, 279: 509-514). Human FEZ1 protein is able to complement the function of UNC-76 in the nematode (Hall A., Science, 1998, 279: 509-514). Coexpression of FEZ1 and the constitutively active mutant of PKCξ induced PC12 cells to undergo neuronal differentiation (Luo L., Nature Rev. Neurosci., 2000, 1:173-180). From these observations, FEZ1 is assumed to play a crucial role in the axon guidance machinery in mammals, although the molecular mechanism involving FEZ1 is still unclear. To determine the regions in FEZ1 and DISC1 involved in their interaction, a yeast two-hybrid assay was performed using various shorter fragments of FEZ1 and DISC1 ((a) of FIG. 3). The C-terminal region of FEZ1 (amino acids 247-392), which is highly conserved with the nematode UNC-76, was required for interaction with DISC1. A DISC1 region (amino acids 446-633), containing two stretches with coiled-coil forming potential and the translocation breakpoint, was shown to be critical for interaction with FEZ1. It is of note that in this assay a DISC1 truncated form (amino acids 348-597) lacking C-terminus downstream of the translocation breakpoint, interacted with FEZ1 weakly, because production of the truncated DISC1 protein would be possible in translocation carriers (Millar J. K., Wilson-Annan J. C., Anderson S., Christie S., Taylor M. S., Semple C. A. M. et al., Hum. Mol. Genet., 2000, 9:1415-1423).

The interaction between DISC1 and FEZ1 was confirmed by an immunoprecipitation assay using HEK293T cells ((b) of FIG. 3). The cells were transfected with FLAG-tagged DISC1 and HA-tagged FEZ1 expression vectors, individually or in combination. Cell lysates were prepared and immunoprecipitated by anti-FLAG or anti-HA antibody.

HA-tagged FEZ1 was detected in the immunoprecipitates by anti-FLAG antibody in Western blot analysis. Conversely, FLAG-tagged DISC1 was detected in the immunoprecipitates by anti-HA antibody. FEZ1 also co-immunoprecipitated with a DISC1 fragment (amino acids 446-633), identified as FEZ1-binding region by the yeast two-hybrid assay, but not with a deleted DISC3 that lacks the binding region. These results demonstrated the interaction between DISC1 and FEZ1 in mammalian cells.

### Example 4

### Intracellular Localization of DISC1 and FEZ1)

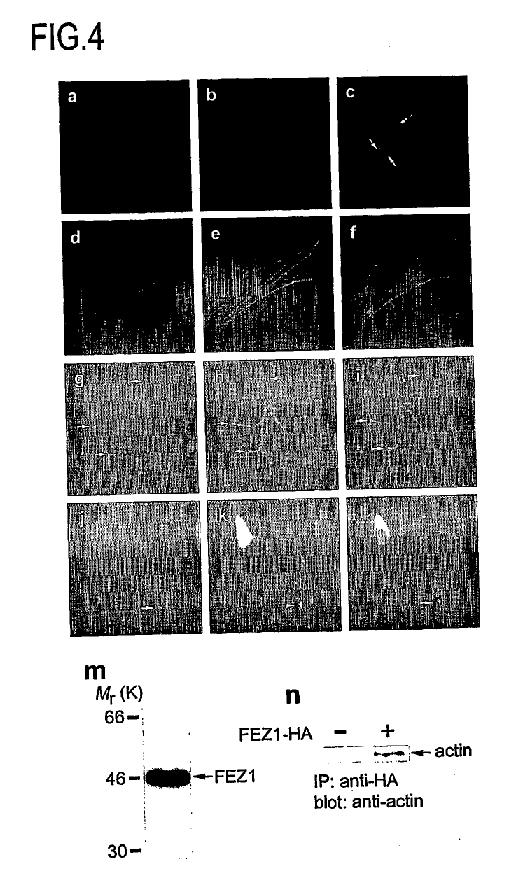

DISC1 has restricted structural similarities to structural proteins (Millar J. K., Wilson-Annan J. C., Anderson S., Christie S., Taylor M. S., Semple C. A. M. et al., Hum. Mol. Genet., 2000, 9:1415-1423). On the other hand, in the course of characterization of FEZ1, a pull-down assay using rat brain lysate revealed that FEZ1 interacts with actin (T. F. and S. K., unpublished data). We next examined the intracellular localization of DISC1 and FEZ1 from the viewpoint of cytoskeletal structure. We raised an antibody against FEZ1 which detected a protein of 46 K in the lysate from SK-N—SH cells ((m) of FIG. 4), the size revealed by in vitro synthesis (Luo L., Nature Rev. Neurosci., 2000, 1:173-180). DISC1 exhibited a punctate distribution in the cytosol of SK-N-SH cells with the perinuclear high density region ((a) of FIG. 4). DISC1 was also located on some filamentous structures which overlapped with F-actin as stress fibers detected by phalloidin staining ((a) to (c) of FIG. 4). FEZ1 was either punctate stained or distributed along organized filamentous structures, which remarkably overlapped with stress fibers, in the cytosol of SK-N-SH cells ((d) to (f) of FIG. 4). In cultured rat hippocampal neurons, colocalization of FEZ1 and F-actin was apparent in neurite growth cones ((g) to (i) of FIG. 4), where F-actin forms lamellipodia and filopodia, dynamic structures involved in axonal extension (Weinberger D. R., Arch. Gen. Psychiatry, 1987, 44: 660-669; and Lewis D. A., Levitt P., Annu. Rev. Neurosci., 2002, 25: 409-432). Transfected GFP-fused DISC1 also colocalized with FEZ1 in the growth cone ((J) to (1) of FIG. 4). These results suggest that the interaction of DISC1 and FEZ3 is associated with F-actin, presumably by direct binding of FEZ1 to actin. The interaction between FEZ1 and actin was confirmed by an immunoprecipitaton assay ((n) of FIG. 4). PC12 cells were transfected with HA-tagged FEZ1 or a mock, and then cell lysates were immunoprecipitated by anti-HA antibody. Co-immunoprecipitation of actin and FEZ1 was detected by the blotting using an anti-actin antibody.

### Example 5

### DISC1 Participates in Neurite Outgrowth through its Interaction with FEZ1)

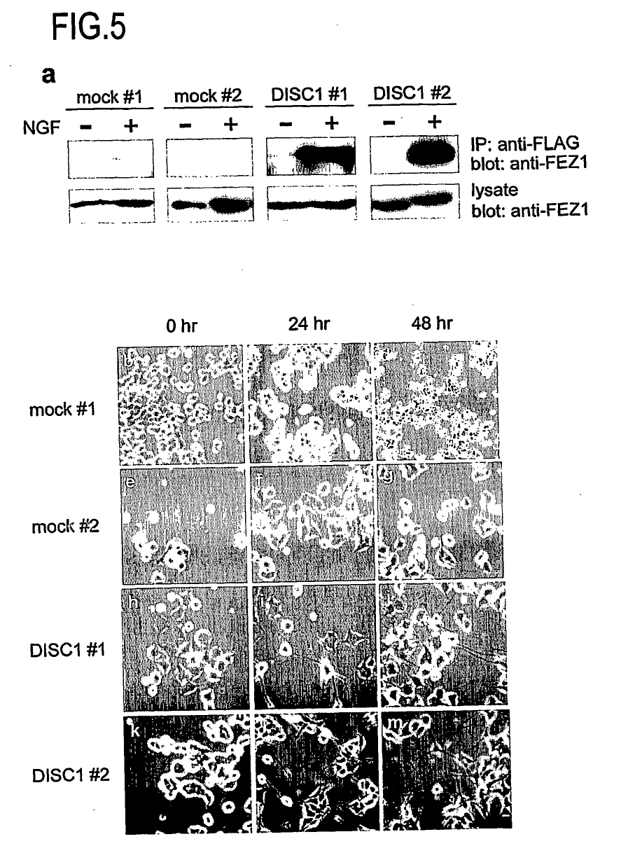

FEZ1 is reported to be involved in axonal outgrowth and fasciculation (Hall A., Science, 1998, 279: 509-514; and Luo L., Nature Rev. Neurosci., 2000, 1:173-180) and we have shown that FEZ1 colocalizes with F-actin. Reorganization of the actin-based cytoskeletal structure is required for neurite outgrowth of neurons (Lewis D. A., Levitt P., Annu. Rev. Neurosci., 2002, 25: 409-42). We evaluated the physiological role of DISC1/FEZ1 interaction in neuronal cells, especially at the stage of neurite outgrowth using PC12 cells. After stimulation with nerve growth factor (NGF), PC12 cells stop proliferation and begin to extend neurites. This feature is widely used as a model system for neuronal differentiation and neurite outgrowth. We established PC12 cell lines stably expressing FLAG-tagged DISC1 and then examined the interaction between FLAG-tagged DISC1 and endogenous FEZ1 in the course of neuronal differentiation. As shown in (a) of FIG. 5, the amount of FEZ1 in the immunoprecipitates by anti-FLAG antibody was drastically increased upon NGF stimulation. As NGF stimulation did not alter the expression levels of endogenous FEZ1 ((a) of FIG. 5, lower panel) and FLAG-tagged DISC1 (data not shown), this result indicates that DISC1/FEZ1 interaction was up-regulated during neuronal differentiation. In mock-stable cells, FEZ1 was not immunoprecipitated ((a) of FIG. 5). When stimulated with NGF, DISC1-stable lines exhibited enhanced neurite extension compared to mock-stable cells ((b) to (m) of FIG. 5).

In similar stimulus experiments using BDNF, serum removal, or PACAP, the DISC1/FEZ1 interaction was up-regulated during neuronal differentiation. In mock stable cells, such a change was not observed. When stimulated by BDNF, serum removal, or PACAP, DISC1 stable cell lines exhibited enhanced neurite outgrowth as compare to mock stable cells.

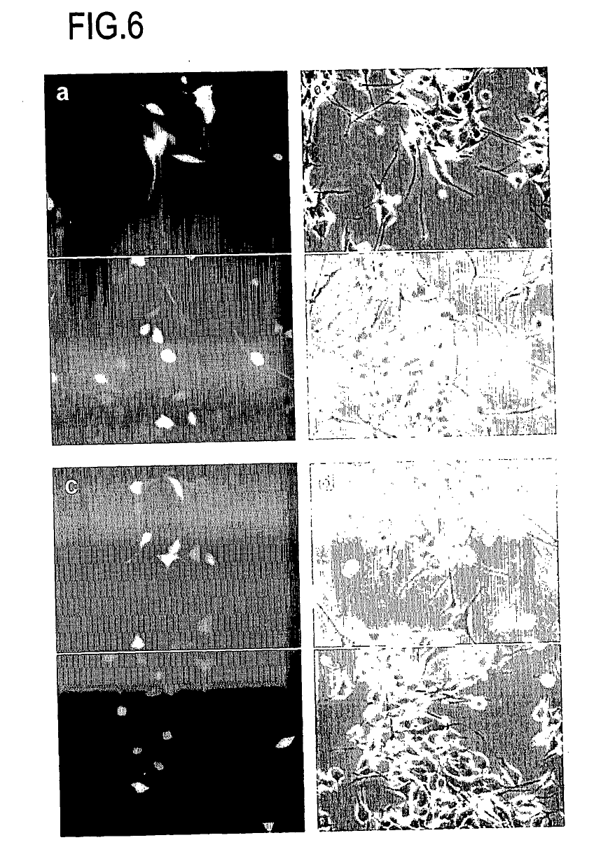

As shown in FIG. 3, a DISC1 region (amino acids 446-633) is essential for the interaction with FEZ1 and therefore is expected to function as a dominant-negative form of DISC1 through the inhibition of binding of FEZ1 and full-length DISC1. This region was cloned into a bicistronic expression vector and transfected into DISC1-stable cells. GFP-labelled cells expressing this region displayed inhibited neurite extension upon NGF stimulation ((c) and (d) of FIG. 6), while no effects of mock-transfection were observed ((a) and (b) of FIG. 6). These results suggest that the interaction of DISC1 and FEZ1 plays a crucial role in neurite outgrowth.

### Example 6

### Identification of an Agent Regulating Interaction Between DISC1 and FEZ1)

Next, the present inventors identified an agent regulating the interaction between DISC1 and FEZ1. About 1000 compounds were tested to achieve the present invention. PC12 cell lines were stimulated by various compounds. Thereafter, when the PC12 cells were stimulated by a certain compound, begun to extend neurites, the compound was selected as a first candidate compound. Next, the resultant candidate compounds were further tested. Cells stimulated by the candidate compound were compared with cells with no stimulus. A compound which enhanced or reduced the interaction between DISC1 and FEZ1 was identified as an agent regulating the DISC1/FEZ1 interaction. As a control experiment, PC12 cell lines stably expressing DISC1 were stimulated with NGF. A change in the DISC1/FEZ1 interaction was detected by immunoprecipitation and Western blotting using monoclonal antibodies against DISC1 and FEZ1. With the above-described method, an agent regulating the interaction between DISC1 and FEZ1 was identified.

### Example 7

### Association of Expression Behavior of DISC1 with KIAA0844)

The present inventors conducted experiments to investigate the expression behavior of DISC1 and associated molecules. The following protocol was used.

(Plasmids)

DISC1-HA (SEQ ID NO. 15): a human DISC1 sequence linked with a Hemaglutinin (HA) sequence at the carboxy terminus thereof (3′ end of a DISC1 DNA sequence) was subcloned into pcDNA3.1 (+).

KIAA-GFP (SEQ ID NO. 17): a human KIAA0844 sequence (SEQ ID NO. 13) linked with a Green Fluorescence Protein (GFP) sequence at the carboxy terminus thereof (3′ end of KIAA0844 DNA sequence) was subcloned into pcDNA3.1 (+) or an adenovirus vector.

(Cell Culture and Gene Introduction)

PC12 cells: rat melanocytoma, PC12 cells, were cultured in DMEM medium supplemented with 10% fetal calf serum and 5% horse serum. The PC12 cells were kindly provided by Dr. Akemichi Baba (Osaka University Graduate School of Pharmaceutices, Japan), though commercially-available PC12 cells (e.g., ATCC No. CRL-1721) can be used to conduct similar experiments.

DISC1-HA structural expression cell (Miyoshi et al., MolPsychiat., 2003): the above-described pcDNA3 (+)-DISC1-HA plasmid was introduced into the above-described PC12 cells using lipofectamin 2000 (Invitrogen), followed by selective culture in 800 μg/ml G418 (Geneticin: Invitrogen). Several single clones formed by surviving cells were collected, and designated as #1, #2, . . . , and so on. Western blotting was performed to detect cells which consecutively expressed DISC1 protein, which were preserved and designated as DISC1-HA structural expression cells.

Gene introduction was performed using lipofectamin 2000 (Invitrogen), or a plasmid subcloned into an adenovirus vector. A gene of interest was introduced into cells by infecting the cells with the adenovirus vector in accordance with the manufacturer's instructions (ViraPower™ Adenoviral Expression System, Invitrogen).

(DISC1-KIAA Interaction Under Stimulation with PACAP and NGF)

DISC1-HA or mock structural expression PC12 cells were plated into 10-cm diameter dishes. After 48 hours, adenovirus KIAA-GFP (about 20 moi) was added at a concentration of 100 μl/10-cm dish.

The medium within the dish was replaced with serum-free medium (horse serum (HS) 1%, DMEM) 24 hours after infection with the adenovirus.

Nerve growth factor (NGF: 50 ng/ml) (Upstate, Inc.), a pituitary adenylate cyclase activating polypeptide (Peptide Institute, Osaka, Japan) (PACAP: 10⁻⁷ M), or both (NGF+PACAP) were added to the medium 4 hours after replacement of the serum-free medium.

Following stimulation with the above-described neurotrophic factor for 24 hours, the cells were solubilized in 1 ml of buffer (NP401%/TNE (20 mM Tris-HCl pH 7.5, 150 mM NaCl, 1 mM EDTA, including a protease inhibitor)), and recovered.

(Immunoprecipitation)

HA antibodies (αHA: Santa Cruz Biotechnology) or GFP antibodies (αGFP: Santa Cruz Biotechnology) (5 μl) were added per 1 ml of solubilizing solution, followed by incubation at 4° C. overnight while rocking.

Protein G-Sephrose beads (Amersham Biosciences) were added to each tube (35 μl), followed by incubation at 4° C. for 3 hours while rocking. Thereafter, the beads were washed 5 times with 1 ml of TNE buffer.

The beads were boiled in a reducing Laemmli SDS sample buffer (provided by the manufacturer) at 95° C. for 5 min, followed by centrifugation. The resultant supernatant was recovered and subjected as a sample to electrophoresis.

(Western Blotting)

A Tris-glycin SDS-polyacrylamide gel (produced from Actrilamide available from Sigma) was used for electrophoresis. After electrophoresis, the sample was transferred to a PVDF membrane (Millipore, Inc.). Blocking was performed with 5% skimmed milk for 30 min. GFP antibodies ( 1/2000) (Santa Cruz Biotechnology) and HA antibodies ( 1/1000) (Sigma Aldrich) were added, followed by incubation at 4° C. while rocking.

The membrane was washed 5 times with PBS-T (0.1 M PBS (0.1 M phosphate buffer, 9 g/L NaCl)+0.1% Tween20) solution for 15 min per wash.

α-mouse IgG HRP-conjugated antibodies (Cell Signaling Technology) ( 1/10000) were added, followed by incubation at 4° C. for 2 hours while rocking.

The membrane was washed 5 times with PBS-T (0.1 M PBS (0.1 M phosphate buffer, 9 g/L NaCl)+0.1% Tween20) solution for 15 min per wash.

A ECL kit (Amersham Biosciences) was used for visualization In accordance with the manufacturer's recommended protocol.

(Results)

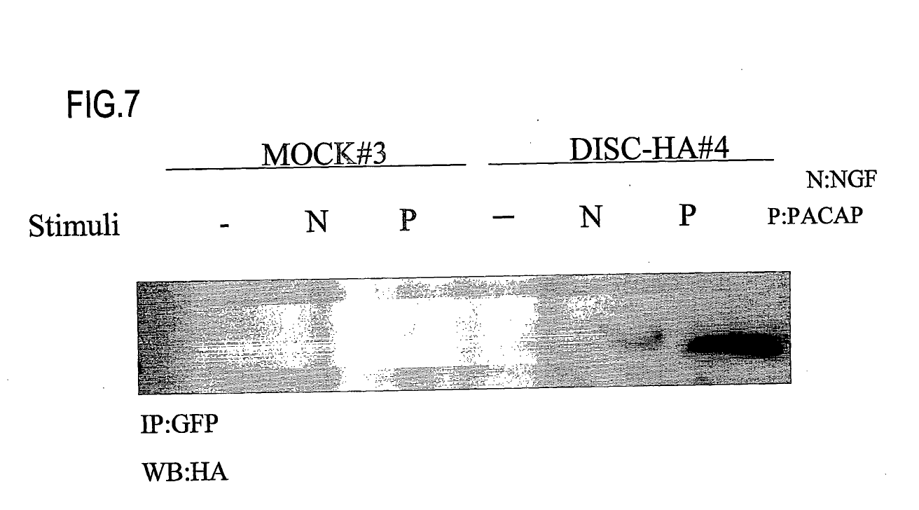

The results are shown in FIG. 7. As shown In FIG. 7, whereas no molecule interacting with DISC1 was found in the mock transfectants, a molecule which was induced by stimulation with NGF and/or PACAP in the DISC1-HA-infected cells was observed. The molecule was identified as KIAA0844.

### Example 8

### Expression Level of DISC1-HA Under Stimulation of NGF and PACAP)

Next, it was determined whether or not the expression of DISC1 was enhanced by stimulation with nerve-relevant molecules, such as NGF, PACAP, and the like. The protocol was described below.

A cell, a plasmid, a gene introduction technique, and a drug stimulation technique as described in Example 7 were used. Immunoprecipitation (IP) was performed using the above-described αHA antibodies. Detection (WB) was performed using αHA antibodies.

(Results)

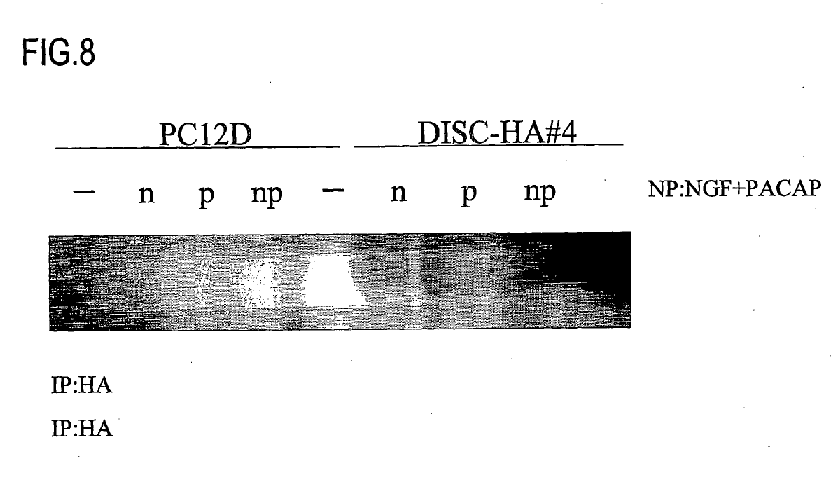

As shown in FIG. 8, DISC1 expression was significantly increased.

### Example 9

### Expression Level of KIAA-GFP Under Stimulation with NGF/PACAP)

Proteins whose expression was changed by infection with DISC1 were investigated by the two-hybrid method. As a result, KIAA0844 was identified. Based on this finding, it was determined whether or not the level of expression of KIAA0844 was changed by stimulation with a nerve relevant agent. The protocol is described below.

A cell, a plasmid, a gene introduction technique, and a drug stimulation technique as described in Examples 7 and 8 were used. Immunoprecipitation (IP) was performed using the above-described αGFPantibodies. Detection (WB) was performed using αGFP antibodies.

(Results)

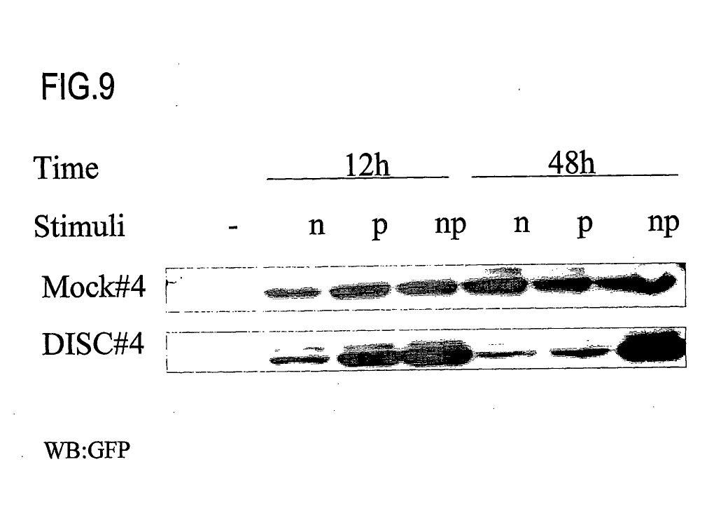

As shown in FIG. 9, the expression of KIAA0844 was enhanced by stimulation with NGF and PACAP. The level of the expression was equivalent to that of DISC1 in Examples 7 and 8. Therefore, it was demonstrated that KIAA0844 was also associated with a condition, disorder or disease associated with a level of axon outgrowth and/or fasciculation, as well as DISC1.

### Example 10

### Analysis of the Amount of KIAA0844 RNA Under Stimulation with NGF/PACAP)

Next, it was similarly determined whether or not the level of mRNA transcription of KIAA0844 was increased by stimulation with a nerve relevant factor. The protocol is described below.

DISC1-HA or mock structural expression PC12 cells were plated into 10-cm diameter dishes. After 48 hours, adenovirus KIAA-GFP (about 20 moi) was added at a concentration of 100 μl/10-cm dish.

The medium of the dish was replaced with serum-free medium (horse serum (HS) 1%, DMEM) 24 hours after infection with the adenovirus.

A nerve growth factor (NGF: 50 ng/ml), a pituitary adenylate cyclase activating polypeptide (PACAP: 10−7 M), or both (NGF+PACAP) were added to the medium 4 hours after replacement of the serum-free medium.

Following stimulation with the above-described drug for 24 hours, β-mercaptoethanol (final concentration: 1%) was added to RLT solution accompanying with the RNeasy Mini Kit (Qiagen), and the cells were recovered.

The RNeasy Mini Kit (Qiagen) was used to isolate RNA in accordance with the manufacturer's recommended protocol.

A formalin denatured gel (Agarose 1%, MOPS 0.02 M, formaldehyde 18%, ethidium bromide 1 μg/ml) was used for electrophoresis.

After electrophoresis, the sample was transferred to nylon membrane, Immobilon-Ny+ (Millipore) in accordance with conventional protocol.

A partial sequence of KIAA0844 (SEQ ID NO. 19) was labeled with ³²P-RI and a probe was synthesized using the ReadyprimeII Random Prime Labelling. System (Amersham Biosciences). The sample was purified, followed by Northern blotting in accordance with conventional protocol.

(Results)

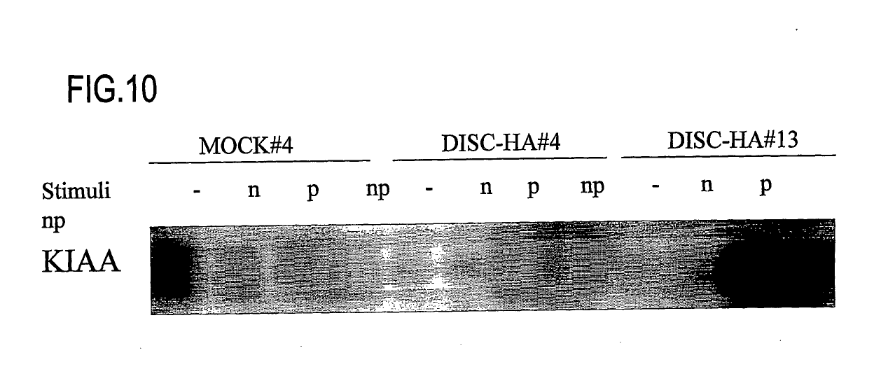

As shown in FIG. 10, it was demonstrated that mRNA molecules of KIAA0844 were induced by stimulation with NGF and PACAP.

Although certain preferred embodiments have been described herein, it is not intended that such embodiments be construed as limitations on the scope of the invention except as set forth in the appended claims. Various other modifications and equivalents will be apparent to and can be readily made by those skilled in the art, after reading the description herein, without departing from the scope and spirit of this invention. All patents, published patent applications and publications cited herein are incorporated by reference as if set forth fully herein.

### INDUSTRIAL APPLICABILITY

The present invention provides an agent which is important for the diagnosis, treatment and prophylaxis of psychiatric diseases, such as schizophrenia and the like, thereby realizing diagnosis, treatment and prophylaxis which cannot be achieved by conventional techniques. Therefore, the present invention is useful in industries of production, search and the like of pharmaceuticals.

1. An agent specifically interacting with:
(a) a polynucleotide having a base sequence set forth in SEQ ID NO. 1 or a fragment thereof;
(b) a polynucleotide encoding a polypeptide consisting of an amino acid sequence set forth in SEQ ID NO. 2 or a fragment thereof;
(c) a polynucleotide encoding a variant polypeptide having an amino acid sequence set forth in SEQ ID NO. 2 or a fragment thereof, wherein at least one amino acid in the sequence has a mutation selected from the group consisting of substitution, addition, and deletion and wherein the variant polypeptide has biological activity;
(d) a polynucleotide, which is a spliced mutant or alleic mutant of a base sequence set forth in SEQ ID NO. 1;
(e) a polynucleotide encoding a species homolog of a polypeptide consisting of an amino acid sequence set forth in SEQ ID NO. 2;
(f) a polynucleotide hybridizable to any one of the polynucleotides of (a) to (e) under stringent conditions and encoding a polypeptide having biological activity; or
(g) a polynucleotide consisting of a base sequence having at least 70% identity to any one of the polynucleotides (a) to (e) or a complementary sequence thereof, and encoding a polypeptide having biological activity.

2. An agent according to claim 1, wherein the agent is selected from the group consisting of a nucleic acid molecule, a polypeptide, a lipid, a sugar chain, a low molecular weight organic molecule, and a composite molecule thereof.

3. An agent according to claim 1, wherein the agent is a nucleic acid molecule of at least 8 contiguous nucleotides in length.

4. An agent according to claim 1, wherein the agent is a nucleic acid molecule having a sequence having at least 70% identity to the nucleic acid sequence of any one of the polynucleotides of (a) to (g).

5. An agent according to claim 1, wherein the agent is a nucleic acid molecule hybridizable to the nucleic acid sequence of any one of the polynucleotides of (a) to (g) under stringent conditions.

6. An agent according to claim 3, wherein the agent is used as a primer.

7. An agent according to claim 3, wherein the agent is used as a probe.

8. An agent according to claim 1, wherein the polynucleotide or the polypeptide comprises a range encoding nucleotides 1095 to 2615 of SEQ ID NO. 1 or a range of amino acids 348 to 854 of SEQ ID NO. 2.

9. An agent according to claim 1, wherein the polynucleotide or the polypeptide comprises a range encoding a range selected from the group consisting of nucleotides 1095 to 1844, nucleotides 1845 to 2615, nucleotides 1095 to 1952, nucleotides 1095 to 1652, nucleotides 1653 to 1952, nucleotides 1391 to 1652, and nucleotides 1391 to 1952 of SEQ ID NO. 1, or a range selected from the group consisting of amino acids 348 to 597, amino acids 598 to 854, amino acids 348 to 633, amino acids 348 to 533, amino acids 534 to 633, amino acids 446 to 533, and amino acids 446 to 633 of SEQ ID NO. 2.

10. An agent according to claim 1, wherein the agent is labeled or labelable.

11. An agent specifically interacting with a polypeptide, wherein the polypeptide comprises:
(a) a polypeptide consisting of an amino acid sequence set forth in SEQ ID NO. 2 or a fragment thereof;
(b) a polypeptide having an amino acid sequence set forth in SEQ ID NO. 2 or a fragment thereof, wherein at least one amino acid in the sequence has a mutation selected from the group consisting of substitution, addition, and deletion and wherein the polypeptide has biological activity;
(c) a polypeptide encoded by a spliced mutant or alleic mutant of a base sequence set forth in SEQ ID NO. 1;
(d) a polypeptide being a species homolog of an amino acid sequence set forth in SEQ ID NO. 2; or
(e) a polypeptide having an amino acid sequence having at least 70% identity to any one of the polypeptides (a) to (d), and having biological activity.

12. An agent according to claim 11, wherein the agent is selected from the group consisting of a nucleic acid molecule, a polypeptide, a lipid, a sugar chain, a low molecular weight organic molecule, and a composite molecule thereof.

13. An agent according to claim 11, wherein the agent is an agent or a derivative thereof.

14. An agent according to claim 11, wherein the agent is used as a probe.

15. An agent according to claim 11, wherein the polypeptide comprises a range of amino acids 348 to 854 of SEQ ID NO. 2.

16. An agent according to claim 11, wherein the polypeptide comprises a range selected from the group consisting of amino acids 348 to 597, amino acids 598 to 854, amino acids 348 to 633, amino acids 348 to 533, amino acids 534 to 633, amino acids 446 to 533, and amino acids 446 to 633 of SEQ ID NO. 2.

17. An agent according to claim 11, wherein the agent is labeled or labelable.

18. An agent specifically interacting with:
(a) a polynucleotide having a base sequence set forth in SEQ ID NO. 3 or a fragment thereof;
(b) a polynucleotide encoding a polypeptide consisting of an amino acid sequence set forth in SEQ ID NO. 4 or a fragment thereof;
(c) a polynucleotide encoding a variant polypeptide having an amino acid sequence set forth in SEQ ID NO. 4 or a fragment thereof, wherein at least one amino acid in the sequence has a mutation selected from the group consisting of substitution, addition, and deletion and wherein the variant polypeptide has biological activity;
(d) a polynucleotide, which is a spliced mutant or alleic mutant of a base sequence set forth in SEQ ID NO. 3;
(e) a polynucleotide encoding a species homolog of a polypeptide consisting of an amino acid sequence set forth in SEQ ID NO. 4;
(f) a polynucleotide hybridizable to any one of the polynucleotides of (a) to (e) under stringent conditions and encoding a polypeptide having biological activity; or
(g) a polynucleotide consisting of a base sequence having at least 70% identity to any one of the polynucleotides (a) to (e) or a complementary sequence thereof, and encoding a polypeptide having biological activity.

19. An agent according to claim 18, wherein the agent is selected from the group consisting of a nucleic acid molecule, a polypeptide, a lipid, a sugar chain, a low molecular weight organic molecule, and a composite molecule thereof.

20. An agent according to claim 18, wherein the agent is a nucleic acid molecule of at least 8 contiguous nucleotides in length.

21. An agent according to claim 18, wherein the agent is a nucleic acid molecule having a sequence having at least 70% identity to the nucleic acid sequence of any one of the polynucleotides of (a) to (g).

22. An agent according to claim 18, wherein the agent is a nucleic acid molecule hybridizable to the nucleic acid sequence of any one of the polynucleotides of (a) to (g) under stringent conditions.

23. An agent according to claim 20, wherein the agent is used as a primer.

24. An agent according to claim 20, wherein the agent is used as a probe.

25. An agent according to claim 18, wherein the polynucleotide or the polypeptide comprises a range encoding nucleotides 478 to 1269 of SEQ ID NO. 3 or a range of amino acids 129 to 392 of SEQ ID NO. 4.

26. An agent according to claim 18, wherein the polynucleotide or the polypeptide comprises a range encoding nucleotides 832 to 1269 of SEQ ID NO. 3 or a range of amino acids 247 to 392 of SEQ ID NO. 4.

27. An agent according to claim 18, wherein the agent is labeled or labelable.

28. An agent specifically interacting with a polypeptide, wherein the polypeptide comprises:
(a) a polypeptide consisting of an amino acid sequence set forth in SEQ ID NO. 4 or a fragment thereof;
(b) a polypeptide having an amino acid sequence set forth in SEQ ID NO. 4 or a fragment thereof, wherein at least one amino acid in the sequence has a mutation selected from the group consisting of substitution, addition, and deletion and wherein the polypeptide has biological activity;
(c) a polypeptide encoded by a spliced mutant or alleic mutant of a base sequence set forth in SEQ ID NO. 3:
(d) a polypeptide being a species homolog of an amino acid sequence set forth in SEQ ID NO. 4; or
(e) a polypeptide having an amino acid sequence having at least 70% identity to any one of the polypeptides (a) to (d), and having biological activity.

29. An agent according to claim 28, wherein the agent is selected from the group consisting of a nucleic acid molecule, a polypeptide, a lipid, a sugar chain, a low molecular weight organic molecule, and a composite molecule thereof.

30. An agent according to claim 28, wherein the agent is an agent or a derivative thereof.

31. An agent according to claim 28, wherein the agent is used as a probe.

32. An agent according to claim 28, wherein the polypeptide comprises a range of amino acids 129 to 392 of SEQ ID NO. 4.

33. An agent according to claim 28, wherein the polypeptide comprises a range of amino acids 247 to 392 of SEQ ID NO. 4.

34. An agent according to claim 28, wherein the agent is labeled or labelable.

35. A composition for determining a function of FEZ1 or KIAA0844, comprising:
(A) an agent specifically interacting with:
(a) a polynucleotide having a base sequence set forth in SEQ ID NO. 1 or a fragment thereof;
(b) a polynucleotide encoding a polypeptide consisting of an amino acid sequence set forth in SEQ ID NO. 2 or a fragment thereof;
(c) a polynucleotide encoding a variant polypeptide having an amino acid sequence set forth in SEQ ID NO. 2 or a fragment thereof, wherein at least one amino acid in the sequence has a mutation selected from the group consisting of substitution, addition, and deletion and wherein the variant polypeptide has biological activity;
(d) a polynucleotide, which is a spliced mutant or alleic mutant of a base sequence set forth in SEQ ID NO. 1;
(e) a polynucleotide encoding a species homolog of a polypeptide consisting of an amino acid sequence set forth in SEQ ID NO. 2;
(f) a polynucleotide hybridizable to any one of the polynucleotides of (a) to (e) under stringent conditions and encoding a polypeptide having biological activity; or
(g) a polynucleotide consisting of a base sequence having at least 70% identity to any one of the polynucleotides (a) to (e) or a complementary sequence thereof, and encoding a polypeptide having biological activity; and/or
(B) an agent specifically interacting with a polypeptide, wherein the polypeptide comprises:
(a) a polypeptide consisting of an amino acid sequence set forth in SEQ ID NO. 2 or a fragment thereof;
(b) a polypeptide having an amino acid sequence set forth in SEQ ID NO. 2 or a fragment thereof, wherein at least one amino acid in the sequence has a mutation selected from the group consisting of substitution, addition, and deletion and wherein the polypeptide has biological activity:
(c) a polypeptide encoded by a spliced mutant or alleic mutant of a base sequence set forth in SEQ ID NO. 1;
(d) a polypeptide being a species homolog of an amino acid sequence set forth in SEQ ID NO. 2; or
(e) a polypeptide having an amino acid sequence having at least 70% identity to any one of the polypeptides (a) to (d), and having biological activity.

36. A composition for determining a function of DISC1 or KIAA0844, comprising:
(A) an agent specifically interacting with:
(a) a polynucleotide having a base sequence set forth in SEQ ID NO. 3 or a fragment thereof;
(b) a polynucleotide encoding a polypeptide consisting of an amino acid sequence set forth in SEQ ID NO. 4 or a fragment thereof;
(c) a polynucleotide encoding a variant polypeptide having an amino acid sequence set forth in SEQ ID NO. 4 or a fragment thereof, wherein at least one amino acid in the sequence has a mutation selected from the group consisting of substitution, addition, and deletion and wherein the variant polypeptide has biological activity;
(d) a polynucleotide, which is a spliced mutant or alleic mutant of a base sequence set forth in SEQ ID NO. 3;
(e) a polynucleotide encoding a species homolog of a polypeptide consisting of an amino acid sequence set forth in SEQ ID NO. 4;
(f) a polynucleotide hybridizable to any one of the polynucleotides of (a) to (e) under stringent conditions and encoding a polypeptide having biological activity; or
(g) a polynucleotide consisting of a base sequence having at least 70% identity to any one of the polynucleotides (a) to (e) or a complementary sequence thereof, and encoding a polypeptide having biological activity; and/or
(B) an agent specifically interacting with a polypeptide, wherein the polypeptide comprises:
(a) a polypeptide consisting of an amino acid sequence set forth in SEQ ID NO. 4 or a fragment thereof;
(b) a polypeptide having an amino acid sequence set forth in SEQ ID NO. 4 or a fragment thereof, wherein at least one amino acid in the sequence has a mutation selected from the group consisting of substitution, addition, and deletion and wherein the polypeptide has biological activity;
(c) a polypeptide encoded by a spliced mutant or alleic mutant of a base sequence set forth in SEQ ID NO. 3;
(d) a polypeptide being a species homolog of an amino acid sequence set forth in SEQ ID NO. 4; or
(e) a polypeptide having an amino acid sequence having at least 70% identity to any one of the polypeptides (a) to (d), and having biological activity.

37. A composition for determining a level of axon outgrowth and/or fasciculation, or a condition, disorder or disease associated with the level of axon outgrowth and/or fasciculation, comprising:
(A) an agent specifically interacting with:
(a) a polynucleotide having abase sequence set forth in SEQ ID NO. 1 or a fragment thereof;
(b) a polynucleotide encoding a polypeptide consisting of an amino acid sequence set forth in SEQ ID NO. 2 or a fragment thereof;
(c) a polynucleotide encoding a variant polypeptide having an amino acid sequence set forth in SEQ ID NO. 2 or a fragment thereof, wherein at least one amino acid in the sequence has a mutation selected from the group consisting of substitution, addition, and deletion and wherein the variant polypeptide has biological activity;
(d) a polynucleotide, which is a spliced mutant or alleic mutant of a base sequence set forth in SEQ ID NO. 1;
(e) a polynucleotide encoding a species homolog of a polypeptide consisting of an amino acid sequence set forth in SEQ ID NO. 2;
(f) a polynucleotide hybridizable to any one of the polynucleotides of (a) to (e) under stringent conditions and encoding a polypeptide having biological activity; or
(g) a polynucleotide consisting of a base sequence having at least 70% identity to any one of the polynucleotides (a) to (e) or a complementary sequence thereof, and encoding a polypeptide having biological activity;
(B) an agent specifically interacting with a polypeptide, wherein the polypeptide comprises:
(a) a polypeptide consisting of an amino acid sequence set forth in SEQ ID NO. 2 or a fragment thereof;
(b) a polypeptide having an amino acid sequence set forth in SEQ ID NO. 2 or a fragment thereof, wherein at least one amino acid in the sequence has a mutation selected from the group consisting of substitution, addition, and deletion and wherein the polypeptide has biological activity;
(c) a polypeptide encoded by a spliced mutant or alleic mutant of a base sequence set forth in SEQ ID NO. 1;
(d) a polypeptide being a species homolog of an amino acid sequence set forth in SEQ ID NO. 2; or
(e) a polypeptide having an amino acid sequence having at least 70% identity to any one of the polypeptides (a) to (d), and having biological activity;
(C) an agent specifically interacting with:
(a) a polynucleotide having a base sequence set forth in SEQ ID NO. 3 or a fragment thereof;
(b) a polynucleotide encoding a polypeptide consisting of an amino acid sequence set forth in SEQ ID NO. 4 or a fragment thereof;
(c) a polynucleotide encoding a variant polypeptide having an amino acid sequence set forth in SEQ ID NO. 4 or a fragment thereof, wherein at least one amino acid in the sequence has a mutation selected from the group consisting of substitution, addition, and deletion and wherein the variant polypeptide has biological activity;
(d) a polynucleotide, which is a spliced mutant or alleic mutant of a base sequence set forth in SEQ ID NO. 3;
(e) a polynucleotide encoding a species homolog of a polypeptide consisting of an amino acid sequence set forth in SEQ ID NO. 4:
(f) a polynucleotide hybridizable to any one of the polynucleotides of (a) to (e) under stringent conditions and encoding a polypeptide having biological activity; or
(g) a polynucleotide consisting of a base sequence having at least 70% identity to any one of the polynucleotides (a) to (e) or a complementary sequence thereof, and encoding a polypeptide having biological activity; and/or
(D) an agent specifically interacting with a polypeptide, wherein the polypeptide comprises:
(a) a polypeptide consisting of an amino acid sequence set forth in SEQ ID NO. 4 or a fragment thereof;
(b) a polypeptide having an amino acid sequence set forth in SEQ ID NO. 4 or a fragment thereof, wherein at least one amino acid in the sequence has a mutation selected from the group consisting of substitution, addition, and deletion and wherein the polypeptide has biological activity;
(c) a polypeptide encoded by a spliced mutant or alleic mutant of a base sequence set forth in SEQ ID NO. 3;
(d) a polypeptide being a species homolog of an amino acid sequence set forth in SEQ ID NO. 4; or
(e) a polypeptide having an amino acid sequence having at least 70% identity to any one of the polypeptides (a) to (d), and having biological activity.

38. A composition according to claim 37, wherein the condition, disorder or disease associated with the level of axon outgrowth and/or fasciculation is schizophrenia or mental retardation.

39. A method for diagnosis of a condition, disorder or disease associated with a level of axon outgrowth and/or fasciculation, comprising the steps of:
(a) measuring binding of DISC1 and FEZ1 in a sample derived from a subject; and
(b) comparing a level of the measured binding with a level of normal binding, wherein when the level of the measured binding is lower than the level of the normal binding, the level of axon outgrowth and/or fasciculation is inferior to a normal level thereof.

40. A method according to claim 39, wherein the condition, disorder or disease associated with the level of axon outgrowth and/or fasciculation is schizophrenia or mental retardation.

41. A method for diagnosis of a condition, disorder or disease associated with a level of axon outgrowth and/or fasciculation, comprising the steps of:
(a) measuring binding of DISC1 and FEZ1 in a subject in need of diagnosis of a condition, disorder or disease associated with a level of axon outgrowth and/or fasciculation;
(b) measuring binding of DISC1 and FEZ1 in a normal subject; and
(c) comparing a level of the binding of (a) with a level of the binding of (b),
wherein when the level of the binding of (a) is higher or lower than the level of the binding of (b), the subject is diagnosed as having an abnormality, disorder or disease associated with a level of axon outgrowth and/or fasciculation.

42. A method according to claim 41, wherein the condition, disorder or disease associated with the level of axon outgrowth and/or fasciculation is schizophrenia or mental retardation.

43. A method according to claim 41, wherein in the steps of (a) and (b), an antibody against a first polypeptide having an amino acid sequence having at least 70% homology to a sequence set forth in SEQ ID NO. 2, or a fragment thereof, is used.

44. A method according to claim 41, wherein in the steps of (a) and (b), an antibody against a second polypeptide having an amino acid sequence having at least 70% homology to a sequence set forth in SEQ ID NO. 4, or a fragment thereof, is used.

45. A method according to claim 41, wherein in the steps of (a) and (b), an antibody against a first polypeptide having an amino acid sequence having at least 70% homology to a sequence set forth in SEQ ID NO. 2, or a fragment thereof, and an antibody against a second polypeptide having an amino acid sequence having at least 70% homology to a sequence set forth in SEQ ID NO. 4, or a fragment thereof, are used.

46. A method for detecting in a genetic mutation associated with a condition, disorder or disease associated with a level of axon outgrowth and/or fasciculation, comprising the step of:
detecting in a mutation in a polynucleotide sequence of a DISC1 gene and/or a FEZ1 gene in a sample.

47. A method according to claim 46, wherein the condition, disorder or disease associated with the level of axon outgrowth and/or fasciculation is schizophrenia or mental retardation.

48. A method according to claim 46, wherein the mutation is linked with a condition, disorder or disease associated with a level of axon outgrowth and/or fasciculation.

49. A kit for diagnosis of a condition, disorder or disease associated with a level of axon outgrowth and/or fasciculation, comprising:
(a) a composition according to claim 37; and
(b) instructions,
the instructions describes:
(i) using the composition to measure binding of DISC1 and FEZ1 in a subject in need of diagnosis of the condition, disorder or disease associated with the level of axon outgrowth and/or fasciculation;
(ii) using the composition to measure binding of DISC1 and FEZ1 in a normal subject; and
(iii) comparing a level of the binding of (i) with a level of the binding of (ii),
wherein when the level of the binding of (i) is higher or lower than the level of the binding of (ii), the subject is diagnosed as having an abnormality, disorder or disease associated with a level of axon outgrowth and/or fasciculation.

50. A kit according to claim 49, wherein the condition, disorder or disease associated with the level of axon outgrowth and/or fasciculation is schizophrenia or mental retardation.

51. A kit for detection of a condition, disorder or disease associated with a level of axon outgrowth and/or fasciculation, comprising:
(a) at least 2 primers of at least 10 nucleotides in length, wherein each of the primers comprises:
(i) a polynucleotide having at least 70% homology to a polynucleotide set forth in SEQ ID NO. 1; and
(ii) a primer having at least 70% homology to a polynucleotide encoding an amino acid sequence set forth in SEQ ID NO. 2 and hybridizable to the polynucleotide at different positions in the amino acid sequence under stringent conditions; and
(b) instructions describing using the primer of (a) to detect a nucleic acid sequence in a sample derived from the subject and detect a mutation in the polynucleotide set forth in SEQ ID NO. 1.

52. A kit according to claim 51, wherein the detected nucleic acid sequence is nucleotides 54 to 2615 in SEQ ID NO. 1.

53. A kit for detection of a condition, disorder or disease associated with a level of axon outgrowth and/or fasciculation, comprising:
(a) at least 2 primers of at least 10 nucleotides in length, wherein each of the primers comprises:
(i) a polynucleotide having at least 70% homology to a polynucleotide set forth in SEQ ID NO. 3; and
(ii) a primer having at least 70% homology to a polynucleotide encoding an amino acid sequence set forth in SEQ ID NO. 4 and hybridizable to the polynucleotide at different positions in the amino acid sequence under stringent conditions; and
(b) instructions describing using the primer of (a) to detect a nucleic acid sequence in a sample derived from the subject and detect a mutation in the polynucleotide set forth in SEQ ID NO. 3.

54. A kit according to claim 53, wherein the detected nucleic acid sequence is nucleotides 94 to 1269 in SEQ ID NO. 3.

55. A method for identifying an agent regulating a condition, disorder or disease associated with a level of axon outgrowth and/or fasciculation, comprising:
(a) contacting a first polypeptide having an amino acid sequence having at least 70% homology to SEQ ID NO. 2 or a fragment thereof with a second polypeptide having an amino acid sequence having at least 70% homology to SEQ ID NO. 4 or a fragment thereof in the presence of a test agent; and
(b) comparing a level of binding of the first polypeptide and the second polypeptide with a level of binding therebetween in the absence of the test agent,
wherein when the level of binding in the presence of the test agent is lower than the level of binding in the absence of the test agent, the test agent is a negative-regulatory agent for the condition, disorder or disease associated with the level of axon outgrowth and/or fasciculation; and when the level of binding in the presence of the test agent is higher than the level of binding in the absence of the test agent, the test agent is a positive-regulatory agent for the condition, disorder or disease associated with the level of axon outgrowth and/or fasciculation.

56. A method according to claim 55, wherein the condition, disorder or disease associated with the level of axon outgrowth and/or fasciculation is schizophrenia or mental retardation.

57. A method according to claim 55, wherein the first polypeptide comprises amino acids 446 to 597 in SEQ ID NO. 2.

58. A method according to claim 55, wherein the second polypeptide comprises amino acids 247 to 392 in SEQ ID NO. 4.

59. A method according to claim 57, wherein the second polypeptide comprises amino acids 247 to 392 in SEQ ID NO. 4.

60. A method according to claim 55, wherein the step of (a) comprises contacting a cell expressing the first polypeptide with a cell expressing the second peptide.

61. A regulatory agent, identified by a method according to claim 55.

62. A pharmaceutical composition, comprising a regulatory agent according to claim 61.

63. A method for treatment or prophylaxis of a condition, disorder or disease associated with a level of axon outgrowth and/or fasciculation, comprising the step of:
administrating a pharmaceutical composition according to claim 62 into a subject.

64. A method according to claim 63, wherein the condition, disorder or disease associated with the level of axon outgrowth and/or fasciculation is schizophrenia or mental retardation.

65. An agent specifically interacting with:
(a) a polynucleotide having a base sequence set forth in SEQ ID NO. 13 or a fragment thereof;
(b) a polynucleotide encoding a polypeptide consisting of an amino acid sequence set forth in SEQ ID NO. 14 or a fragment thereof;
(c) a polynucleotide encoding a variant polypeptide having an amino acid sequence set forth in SEQ ID NO. 14 or a fragment thereof, wherein at least one amino acid in the sequence has a mutation selected from the group consisting of substitution, addition, and deletion and wherein the variant polypeptide has biological activity;
(d) a polynucleotide, which is a spliced mutant or alleic mutant of a base sequence set forth in SEQ ID NO. 13;
(e) a polynucleotide encoding a species homolog of a polypeptide consisting of an amino acid sequence set forth in SEQ ID NO. 14;
(f) a polynucleotide hybridizable to any one of the polynucleotides of (a) to (e) under stringent conditions and encoding a polypeptide having biological activity; or
(g) a polynucleotide consisting of a base sequence having at least 70% identity to any one of the polynucleotides (a) to (e) or a complementary sequence thereof, and encoding a polypeptide having biological activity.

66. An agent according to claim 65, wherein the base sequence set forth in SEQ ID NO. 13 is provided as KIAA0844.

67. An agent according to claim 65, wherein the agent is selected from the group consisting of a nucleic acid molecule, a polypeptide, a lipid, a sugar chain, a low molecular weight organic molecule, and a composite molecule thereof.

68. An agent according to claim 65, wherein the agent is a nucleic acid molecule of at least 8 contiguous nucleotides in length.

69. An agent according to claim 65, wherein the agent is a nucleic acid molecule having a sequence having at least 70% identity to the nucleic acid sequence of any one of the polynucleotides of (a) to (g).

70. An agent according to claim 65, wherein the agent is a nucleic acid molecule hybridizable to the nucleic acid sequence of any one of the polynucleotides of (a) to (g) under stringent conditions.

71. An agent according to claim 67, wherein the agent is used as a primer.

72. An agent according to claim 67, wherein the agent is used as a probe.

73. An agent according to claim 65, wherein the agent is labeled or labelable.

74. An agent specifically interacting with a polypeptide, wherein the polypeptide comprises:
(a) a polypeptide consisting of an amino acid sequence set forth in SEQ ID NO. 14 or a fragment thereof;
(b) a polypeptide having an amino acid sequence set forth in SEQ ID NO. 14 or a fragment thereof, wherein at least one amino acid in the sequence has a mutation selected from the group consisting of substitution, addition, and deletion and wherein the polypeptide has biological activity;
(c) a polypeptide encoded by a spliced mutant or alleic mutant of a base sequence set forth in SEQ ID NO. 13;
(d) a polypeptide being a species homolog of an amino acid sequence set forth in SEQ ID NO. 14; or
(e) a polypeptide having an amino acid sequence having at least 70% identity to any one of the polypeptides (a) to (d), and having biological activity.

75. An agent according to claim 74, wherein the base sequence set forth in SEQ ID NO. 13 is provided as KIAA0844.

76. An agent according to claim 74, wherein the agent is selected from the group consisting of a nucleic acid molecule, a polypeptide, a lipid, a sugar chain, a low molecular weight organic molecule, and a composite molecule thereof.

77. An agent according to claim 74, wherein the agent is an agent or a derivative thereof.

78. An agent according to claim 74, wherein the agent is used as a probe.

79. An agent according to claim 74, wherein the agent is labeled or labelable.

80. A composition for determining a function of FEZ1, comprising:
(A) an agent specifically interacting with:
(a) a polynucleotide having a base sequence set forth in SEQ ID NO. 13 or a fragment thereof;
(b) a polynucleotide encoding a polypeptide consisting of an amino acid sequence set forth in SEQ ID NO. 14 or a fragment thereof;
(c) a polynucleotide encoding a variant polypeptide having an amino acid sequence set forth in SEQ ID NO. 14 or a fragment thereof, wherein at least one amino acid in the sequence has a mutation selected from the group consisting of substitution, addition, and deletion and wherein the variant polypeptide has biological activity;
(d) a polynucleotide, which is a spliced mutant or alleic mutant of a base sequence set forth in SEQ ID NO. 13;
(e) a polynucleotide encoding a species homolog of a polypeptide consisting of an amino acid sequence set forth in SEQ ID NO. 14;
(f) a polynucleotide hybridizable to any one of the polynucleotides of (a) to (e) under stringent conditions and encoding a polypeptide having biological activity; or
(g) a polynucleotide consisting of a base sequence having at least 70% identity to any one of the polynucleotides (a) to (e) or a complementary sequence thereof, and encoding a polypeptide having biological activity; and/or
(B) an agent specifically interacting with a polypeptide, wherein the polypeptide comprises:
(a) a polypeptide consisting of an amino acid sequence set forth in SEQ ID NO. 14 or a fragment thereof:
(b) a polypeptide having an amino acid sequence set forth in SEQ ID NO. 14 or a fragment thereof, wherein at least one amino acid in the sequence has a mutation selected from the group consisting of substitution, addition, and deletion and wherein the polypeptide has biological activity;
(c) a polypeptide encoded by a spliced mutant or alleic mutant of a base sequence set forth in SEQ ID NO. 13;
(d) a polypeptide being a species homolog of an amino acid sequence set forth in SEQ ID NO. 14; or
(e) a polypeptide having an amino acid sequence having at least 70% identity to any one of the polypeptides (a) to (d), and having biological activity.

81. A composition for determining a function of DISC1, comprising:
(A) an agent specifically interacting with:
(a) a polynucleotide having a base sequence set forth in SEQ ID NO. 13 or a fragment thereof;
(b) a polynucleotide encoding a polypeptide consisting of an amino acid sequence set forth in SEQ ID NO. 14 or a fragment thereof;
(c) a polynucleotide encoding a variant polypeptide having an amino acid sequence set forth in SEQ ID NO. 14 or a fragment thereof, wherein at least one amino acid in the sequence has a mutation selected from the group consisting of substitution, addition, and deletion and wherein the variant polypeptide has biological activity;
(d) a polynucleotide, which is a spliced mutant or alleic mutant of a base sequence set forth in SEQ ID NO. 13:
(e) a polynucleotide encoding a species homolog of a polypeptide consisting of an amino acid sequence set forth in SEQ ID NO. 14;
(f) a polynucleotide hybridizable to any one of the polynucleotides of (a) to (e) under stringent conditions and encoding a polypeptide having biological activity; or
(g) a polynucleotide consisting of a base sequence having at least 70% identity to any one of the polynucleotides (a) to (e) or a complementary sequence thereof, and encoding a polypeptide having biological activity; and/or
(B) an agent specifically interacting with a polypeptide, wherein the polypeptide comprises:
(a) a polypeptide consisting of an amino acid sequence set forth in SEQ ID NO. 14 or a fragment thereof;
(b) a polypeptide having an amino acid sequence set forth in SEQ ID NO. 14 or a fragment thereof, wherein at least one amino acid in the sequence has a mutation selected from the group consisting of substitution, addition, and deletion and wherein the polypeptide has biological activity;
(c) a polypeptide encoded by a spliced mutant or alleic mutant of a base sequence set forth in SEQ ID NO. 13;
(d) a polypeptide being a species homolog of an amino acid sequence set forth in SEQ ID NO. 14; or
(e) a polypeptide having an amino acid sequence having at least 70% identity to any one of the polypeptides (a) to (d), and having biological activity.

82. A composition for determining a level of axon outgrowth and/or fasciculation, or a condition, disorder or disease associated with the level of axon outgrowth and/or fasciculation, comprising:
(A) an agent specifically interacting with:
(a) a polynucleotide having a base sequence set forth in SEQ ID NO. 13 or a fragment thereof:
(b) a polynucleotide encoding a polypeptide consisting of an amino acid sequence set forth in SEQ ID NO. 14 or a fragment thereof;
(c) a polynucleotide encoding a variant polypeptide having an amino acid sequence set forth in SEQ ID NO. 14 or a fragment thereof, wherein at least one amino acid in the sequence has a mutation selected from the group consisting of substitution, addition, and deletion and wherein the variant polypeptide has biological activity;
(d) a polynucleotide, which is a spliced mutant or alleic mutant of a base sequence set forth in SEQ ID NO. 13;
(e) a polynucleotide encoding a species homolog of a polypeptide consisting of an amino acid sequence set forth in SEQ ID NO. 14;
(f) a polynucleotide hybridizable to any one of the polynucleotides of (a) to (e) under stringent conditions and encoding a polypeptide having biological activity; or
(g) a polynucleotide consisting of a base sequence having at least 70% identity to any one of the polynucleotides (a) to (e) or a complementary sequence thereof, and encoding a polypeptide having biological activity; and/or
(B) an agent specifically interacting with a polypeptide, wherein the polypeptide comprises:
(a) a polypeptide consisting of an amino acid sequence set forth in SEQ ID NO. 14 or a fragment thereof;
(b) a polypeptide having an amino acid sequence set forth in SEQ ID NO. 14 or a fragment thereof, wherein at least one amino acid in the sequence has a mutation selected from the group consisting of substitution, addition, and deletion and wherein the polypeptide has biological activity;
(c) a polypeptide encoded by a spliced mutant or alleic mutant of a base sequence set forth in SEQ ID NO. 13;
(d) a polypeptide being a species homolog of an amino acid sequence set forth in SEQ ID NO. 14; or
(e) a polypeptide having an amino acid sequence having at least 70% identity to any one of the polypeptides (a) to (d), and having biological activity.

83. A composition according to claim 82, wherein the condition, disorder or disease associated with the level of axon outgrowth and/or fasciculation is schizophrenia or mental retardation.

84. A method for diagnosis of a condition, disorder or disease associated with a level of axon outgrowth and/or fasciculation, comprising the steps of:
(a) measuring binding of DISC1 or FEZ1 and KIAA0844 in a sample derived from a subject; and
(b) comparing a level of the measured binding with a level of normal binding, wherein when the level of the measured binding is lower than the level of the normal binding, the level of axon outgrowth and/or fasciculation is inferior to a normal level thereof.

85. A method according to claim 84, wherein the condition, disorder or disease associated with the level of axon outgrowth and/or fasciculation is schizophrenia or mental retardation.

86. A method for diagnosis of a condition, disorder or disease associated with a level of axon outgrowth and/or fasciculation, comprising the steps of:
(a) measuring binding of DISC1 or FEZ1 and KIAA0844 in a subject in need of diagnosis of a condition, disorder or disease associated with a level of axon outgrowth and/or fasciculation;
(b) measuring binding of DISC1 or FEZ1 and KIAA0844 in a normal subject; and
(c) comparing a level of the binding of (a) with a level of the binding of (b),
wherein when the level of the binding of (a) is higher or lower than the level of the binding of (b), the subject is diagnosed as having an abnormality, disorder or disease associated with a level of axon outgrowth and/or fasciculation.

87. A kit for diagnosis of a condition, disorder or disease associated with a level of axon outgrowth and/or fasciculation, comprising:
(a) a composition according to claim 82; and
(b) instructions,
the instructions describes:
(i) using the composition to measure binding of DISC1 and FEZ1 in a subject in need of diagnosis of the condition, disorder or disease associated with the level of axon outgrowth and/or fasciculation;
(ii) using the composition to measure binding of DISC1 and FEZ1 in a normal subject; and
(iii) comparing a level of the binding of (i) with a level of the binding of (ii),
wherein when the level of the binding of (i) is higher or lower than the level of the binding of (ii), the subject is diagnosed as having an abnormality, disorder or disease associated with a level of axon outgrowth and/or fasciculation.

88. A kit for detection of a condition, disorder or disease associated with a level of axon outgrowth and/or fasciculation, comprising:
(a) at least 2 primers of at least 10 nucleotides in length, wherein each of the primers comprises:
(i) a polynucleotide having at least 70% homology to a polynucleotide set forth in SEQ ID NO. 13; and
(ii) a primer having at least 70% homology to a polynucleotide encoding an amino acid sequence set forth in SEQ ID NO. 14 and hybridizable to the polynucleotide at different positions in the amino acid sequence under stringent conditions; and
(b) instructions describing using the primer of (a) to detect a nucleic acid sequence in a sample derived from the subject and detect a mutation in the polynucleotide set forth in SEQ ID NO. 13.
